(19) United States
(12) Patent Application Publication
(10) Pub. No.: US 2025/0101416 A1
(43) Pub. Date: Mar. 27, 2025

(54) COMPOSITIONS AND METHODS FOR TREATMENT OF TRANSTHYRETIN AMYLOIDOSIS

(71) Applicant: ACCUREDIT THERAPEUTICS (SUZHOU) CO., LTD., Suzhou, Jiangsu (CN)

(72) Inventors: Fuxin SHI, Winchester, MA (US); Wenhu Cao, Suzhou, Jiangsu (CN); Ye Chen, Suzhou, Jiangsu (CN); Pengcheng Zhang, Suzhou, Jiangsu (CN); Nafeesa Khatoon, Suzhou, Jiangsu (CN); Huanle Liu, Suzhou, Jiangsu (CN)

(21) Appl. No.: 18/900,481

(22) Filed: Sep. 27, 2024

Related U.S. Application Data
(63) Continuation of application No. PCT/CN2023/083904, filed on Mar. 24, 2023.
Application No. PCT/CN2023/083904 is a continuation of application No. PCT/CN2022/083666, filed on Mar. 29, 2022.

(51) Int. Cl. C12N 15/11; A61K 9/51; C12N 9/22

(52) U.S. Cl. CPC C12N 15/111; A61K 9/5123; C12N 9/22; C12N 2310/20; C12N 2310/315; C12N 2310/321; C12N 2310/322

(57) ABSTRACT

Compositions and methods are described herein for treating subjects having amyloidosis associated with transthyretin (ATTR).

### CROSS REFERENCE TO RELATED APPLICATIONS

This application claims priority to PCT/CN2023/083904 filed Mar. 24, 2023 and PCT/CN2022/083666 filed Mar. 29, 2022, entitled COMPOSITIONS AND METHODS FOR TREATMENT OF TRANSTHYRETIN AMYLOIDOSIS, the contents of which is incorporated by reference herein.

### SEQUENCE LISTING

This application contains a Sequence Listing that has been submitted electronically as an XML file named 53333-0002001.xml. The XML file, created on Dec. 16, 2024, is 389,677 bytes in size. The material in the XML file is hereby incorporated by reference in its entirety.

### TECHNICAL FIELD

This disclosure relates to compositions and methods for the treatment of amyloidosis associated with transthyretin (ATTR).

### BACKGROUND

Transthyretin (TTR) is a transport protein that carries thyroxine and retinol in the plasma. TTR is a homotetrameric protein composed of four monomers of 127 amino acids each. It is primarily synthesized in the liver and minimally in the choroid plexus and retina. Transthyretin amyloidosis (ATTR) is caused by an accumulation of TTR amyloid fibrils deposited extracellularly in various tissues, such as the heart and kidney. ATTR is a systemic disorder and manifests as polyneuropathy, autonomic neuropathy, and cardiomyopathy. Hereditary (familial) ATTR amyloidosis is caused by more than 100 autosomal dominant mutations in the TTR gene, which leads to the accumulation of misfolded monomers that destabilizes and dissociates the TTR tetramer. A wild-type ATTR amyloidosis also has been identified to be caused by misfolding and deposition of wild-type TTR in older males and is associated with heart rhythm problems, heart failure, and carpal tunnel syndrome.

Treatment of ATTR amyloidosis that stop disease progression and improve quality of life are limited (HATTR guide, May 2018 Akcea Therapeutics, TTR-095-2 05/18). While liver transplant has been studied for treatment of ATTR, its use is declining as it involves significant risk and disease progression sometimes continues after transplantation. Small molecule stabilizers, such as diflunisal and tafamidis, appear to slow ATTR progression, but these agents do not halt disease progression. Diflunisal and liver transplantation do not reverse the damage caused by TTR deposits, and both treatments are restricted to relatively healthy patients due to safety and mortality risks. Mickle K. et al., J Manag Care Spec Pharm. 2019; 25 (1): 10-14. Tafamidis is also prohibitively expensive. Gilad A. et al., JACC May 11, 2021; Volume 77, Issue 18.

Approaches using small interfering RNA (siRNA) (such as patisiran), antisense oligonucleotides (inotersin), or a monoclonal antibody targeting amyloid fibrils for destruction are relatively new. While results on short-term suppression of TTR expression show encouraging preliminary data, a need exists for treatments that can produce long-lasting suppression of TTR. Accordingly, efficacious and long-lasting therapies for ATTR are urgently needed.

### SUMMARY

This disclosure relates to compositions and methods to knockout the expression of the TTR gene using CRISPR/Cas system, thereby substantially reducing or eliminating the production of mutant TTR proteins or wildtype TTR proteins in the liver and circulation. This disclosure is based, at least in part, on the findings that novel guide RNA (gRNA) with high editing efficiency can knockout or knock down mutant or wildtype TTR gene expression, thereby offering a long-lasting treatment for ATTR.

In a first aspect, this disclosure features a guide RNA comprising: a. a sequence selected from SEQ ID NOs: 1-47; b. at least 15, 16, 17, 18, 19, or 20 contiguous nucleotides of a sequence selected from SEQ ID NOs: 1-47; or c. a sequence that is at least 90%, 91%, 92%, 93%, 94%, 95%, 96%, 97%, 98%, or 99% identical to a sequence selected from SEQ ID NOs: 1-47.

In a second aspect, this disclosure features a guide RNA comprising: a. a sequence selected from SEQ ID NOs: 48-81 or SEQ ID NOs: 275-307; b. at least 18, 19, or 20 contiguous nucleotides of a sequence selected from SEQ ID NOs: 48-81 or SEQ ID NOs: 275-307; or c. a sequence that is at least 90%, 91%, 92%, 93%, 94%, 95%, 96%, 97%, 98%, or 99% identical to a sequence selected from SEQ ID NOs: 48-81 or SEQ ID NOs: 275-307.

In a third aspect, this disclosure features a vector comprising one or more nucleic acids encoding one or more guide RNAs, wherein the one or more guide RNAs comprise: I. a. one or more sequences selected from SEQ ID NOs: 1-47; I. b. at least 15, 16, 17, 18, 19, or 20 contiguous nucleotides of one or more sequences selected from SEQ ID NOs: 1-47; or I c. one or more sequences that is at least 90%, 91%, 92%, 93%, 94%, 95%, 96%, 97%, 98%, or 99% identical to a sequence selected from SEQ ID NOs: 1-47; or II. a. one or more sequences selected from SEQ ID NOs: 48-81 or SEQ ID NOs: 275-307; II. b. at least 18, 19, or 20 contiguous nucleotides of one or more sequences selected from SEQ ID NOs: 48-81 or SEQ ID NOs: 275-307; or II. c. one or more sequences, each of which is at least 90%, 91%, 92%, 93%, 94%, 95%, 96%, 97%, 98%, or 99% identical to a sequence selected from SEQ ID NOs: 48-81 or SEQ ID NOs: 275-307.

In a fourth aspect, this disclosure features composition comprising (i) a nucleic acid encoding a guide RNA, or a vector comprising the nucleic acid encoding a guide RNA, wherein the guide RNA comprises I. a. a sequence selected from SEQ ID NOs: 1-47; I.b. at least 15, 16, 17, 18, 19, or 20 contiguous nucleotides of a sequence selected from SEQ ID NOs: 1-47; or I.c. a sequence that is at least 90%, 91%, 92%, 93%, 94%, 95%, 96%, 97%, 98%, or 99% identical to a sequence selected from SEQ ID NOs: 1-47; or II. a. a sequence selected from SEQ ID NOs: 48-81 or SEQ ID NOs: 275-307; II. b. at least 18, 19, or 20 contiguous nucleotides of a sequence selected from SEQ ID NOs: 48-81 or SEQ ID NOs: 275-307; or II. c. a sequence that is at least 90%, 91%, 92%, 93%, 94%, 95%, 96%, 97%, 98%, or 99% identical to a sequence selected from SEQ ID NOs: 48-81 or SEQ ID NOs: 275-307; and (ii) an RNA-guided DNA binding agent, a nucleic acid encoding an RNA-guided DNA binding agent, or a vector comprising the nucleic acid encoding an RNA-guided DNA binding agent.

In a fifth aspect, the disclosure features a method of modifying the human transthyretin (TTR) gene and/or inducing a double-stranded break (DSB) within the TTR gene, comprising administering the composition of the disclosure to a cell, wherein the composition recognizes and cleaves a TTR target sequence.

In a sixth aspect, the disclosure features a method of reducing TTR serum concentration, reducing or preventing the deposition of amyloid or amyloid fibrils, and/or treating transthyretin amyloidosis (ATTR) in a subject, comprising administering the composition of the disclosure to a cell to the subject in need thereof, wherein the composition recognizes and cleaves a TTR target sequence, thereby reducing TTR serum concentration, reducing or preventing the deposition of amyloids or amyloid fibrils, and/or treating transthyretin amyloidosis (ATTR) in the subject.

In some embodiments, the RNA-guided DNA binding agent comprises a Cas nuclease or a Cas nickase. In some embodiments, the guide RNA is a hybrid DNA-RNA guide. In some embodiments, the hybrid DNA-RNA guide includes a sequence selected from SEQ ID NOs: 302 and 303. In some embodiments, the nucleic acid encoding the RNA-guided DNA binding agent is a Cas9 nucleic acid comprising the nucleotide sequence set forth in SEQ ID NO: 273 or 274. In some embodiments, the RNA-guided DNA binding agent is a Cas9 comprising the amino acid sequence set forth in SEQ ID NO: 272. In some embodiments, the Cas nuclease is a Class 2 Cas nuclease. In some embodiments, the Cas nuclease is Cas9, Cpf1, C2c1, C2c2, and C2c3, or a modified protein thereof. In some embodiments, the Cas nuclease is an S. pyogenes or an S. aureus Cas9 nuclease or a modified protein thereof. In some embodiments, the Cas nuclease is from a Type-II CRISPR/Cas system. In some embodiments, the Cas nuclease is fused to an exonuclease. In some embodiments, the exonuclease is selected from the group consisting of TREX1, TREX2, and MRE11. In some embodiments, the exonuclease is a truncated exonuclease. In some embodiments, the Cas nuclease is fused to an exonuclease at the C terminus with a GGGGS (SEQ ID NO:374) linker.

In some embodiments, the compositions of the disclosure are for use in editing of the Transthyretin (TTR) gene. In some embodiments, the editing is calculated as a percentage of a population of cells that is edited (percent editing). In some embodiments, between about 30% and 99% of the population of cells are edited. In some embodiments, the percent editing is between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the population of cells.

In some embodiments, the composition of the disclosure reduces the deposition of amyloids in at least one tissue or organ. In some embodiments, the tissue or organ is liver, stomach, colon, sciatic nerve, or dorsal root ganglion. In some embodiments, the amyloid deposition is determined 8 weeks after administration of the composition. In some embodiments, the amyloid deposition is compared to a negative control or a level determined in the subject before administration of the composition. In some embodiments, the amyloid deposition is reduced by at least 20% relative to that in a corresponding negative control or a level determined in the subject before administration of the composition.

In some embodiments, the composition is administered or delivered at least once. In some embodiments, the administration or delivery occurs at an interval of (a) 1, 2, 3, 4, 5, 6, 7, 8, 9, 10, 11, 12, 13, 14, or 15 days; (b) 1, 2, 3, 4, 5, 6, 7, 8, 9, 10, 11, 12, 13, 14, or 15 weeks; or (c) 1, 2, 3, 4, 5, 6, 7, 8, 9, 10, 11, 12, 13, 14, or 15 months.

In some embodiments, the guide RNA is at least partially complementary to a target sequence present in the human TTR gene. In some embodiments, the target sequence is in exon 1, 2, 3, or 4 of the human TTR gene. In some embodiments, the guide RNA sequence is complementary to a target sequence in the positive strand of the TTR gene. In some embodiments, the guide RNA sequence is complementary to a target sequence in the negative strand of TTR. In some embodiments, the first guide sequence is complementary to a first target sequence in the positive strand of the TTR gene, and wherein the composition further comprises a second guide sequence that is complementary to a second target sequence in the negative strand of the TTR gene.

In some embodiments, the guide RNA comprises a crRNA and further comprises a tracrRNA or a portion thereof, wherein the tracrRNA (trRNA) comprises the nucleotide sequence set forth in SEQ ID NO: 257 wherein the trRNA is operably linked to the crRNA.

In some embodiments, the guide RNA is a dual guide RNA (dgRNA). In some embodiments, the guide RNA is a single guide (sgRNA). In some embodiments, the guide RNA comprises at least one modification. In some embodiments, the at least one modification comprises a 2′-O-methyl (2′-O-Me) modified nucleotide, a phosphorothioate (PS) bond between nucleotides, or a 2′-fluoro (2′-F) modified nucleotide. In some embodiments, the at least one modification comprises a modification at one or more of the first five nucleotides at the 5′ end of the guide RNA and/or one or more of the last five nucleotides at the 3′ end of the guide RNA. In some embodiments, the at least one modification comprises a modification of at least 50% of the nucleotides of the guide RNA.

In some embodiments, the sgRNA comprises a guide sequence that is at least 90% identical to a sequence selected from SEQ ID NOs: 1-81 or SEQ ID NOs: 275-307. In some embodiments, the sgRNA comprises a nucleotide sequence set forth in any one of SEQ ID NOs: 171-251 or SEQ ID NOs: 341-373. In some embodiments, the sgRNA comprises a nucleotide sequence that is at least 90% identical to the nucleotide sequence set forth in any one of SEQ ID NOs: 171-251 or SEQ ID NOs: 341-373. In some embodiments, at least a portion of the sgRNA is a hybrid DNA-RNA guide. In some embodiments, the sgRNA comprises a nucleotide sequence set forth in any one of SEQ ID NOs: 368-369.

In some embodiments, the guide RNA is associated with a lipid nanoparticle (LNP). In some embodiments, the composition is a pharmaceutical formulation and further comprises a pharmaceutically acceptable carrier.

In some embodiments, the composition includes (i) the nucleic acid encoding a guide RNA or the vector comprising the nucleic acid encoding the guide RNA, and (ii) an mRNA encoding the RNA-guided DNA binding agent, which is associated with a lipid nanoparticle (LNP). In some embodiments, the LNP comprises ALC0315, DSPC, cholesterol, and DMG-PEG2000. In some embodiments, the N/P ratio of the LNP is about 1, 2, 3, 4, 5, 6, 7, 8, 9, or 10. In some embodiments, the N/P ratio is about 3-7. In some embodiments, ALC0315 comprises from 40 mol % to 60 mol % of the total lipid present in the particle, DSPC comprises from 5 mol % to 15 mol % of the total lipid present in the particle, cholesterol comprises from 30 mol % to 50 mol % of the total lipid present in the particle, and DMG-PEG2000 comprises from 1 mol % to 5 mol % total lipid present in the particle. In some embodiments, the LNP includes ALC0315, DSPC, cholesterol, and DMG-PEG2000 at a molar ratio of 50:9.5:37.5:3.

In some embodiments, the composition reduces or prevents amyloid deposition and/or formation of amyloid fibrils. In some embodiments, the amyloid deposition and/or formation of amyloid fibrils are in the nerves, heart, or gastrointestinal track.

In some embodiments, administering the composition leads to a deletion or insertion of one or more nucleotide(s) in the TTR gene. In some embodiments, the deletion or insertion of a nucleotide(s) induces a frameshift or nonsense mutation in the TTR gene. In some embodiments, the a frameshift or nonsense mutation is induced in the TTR gene of about 20% to about 30% of cells. In some embodiments, the cells are liver cells. In some embodiments, a deletion or insertion of a nucleotide(s) occurs in the TTR gene at least 50-fold or more than in off-target sites.

In some embodiments, the composition reduces levels of monomeric, dimeric, and/or tetrameric TTR in the subject. In some embodiments, the levels of monomeric, dimeric, and/or tetrameric TTR are reduced by at least 30%. In some embodiments, the levels of monomeric, dimeric and/or tetrameric TTR are measured in serum, plasma, blood, or cerebral spinal fluid.

In some embodiments, the subject has ATTRwt, hereditary ATTR, a family history of ATTR, or familial amyloid polyneuropathy. In some embodiments, the subject exhibits nerve symptoms of ATTR. In some embodiments, the subject has familial amyloid cardiomyopathy. In some embodiments, the subject exhibits cardiac symptoms of ATTR.

In some embodiments, the subject expresses a wild-type TTR or a TTR having one or more mutations selected from the group consisting of the following mutations: V28S, F33V, F33L, K35N, K35T, A36P, D38V, E42G, G47R, G47V, G47E, T49A, S50R, G53E, E54K, E54G, E54Q, T59K, E61K, Y69H, S77Y, S77F, G83R, A97S, Y114C, V30A, V30G, V30L, V30M, T60A, V122A, V122I, or V122(−). In some embodiments, the subject is homozygous for wild-type TTR.

In some embodiments, after administration of the composition of the disclosure, the subject exhibits an improvement, stabilization, or slowing of change in symptoms of sensorimotor neuropathy. In some embodiments, the improvement, stabilization, or slowing of change in sensory neuropathy is measured using electromyogram, nerve conduction tests, or patient-reported outcomes. In some embodiments, the subject exhibits an improvement, stabilization, or slowing of change in symptoms of congestive heart failure.

In some embodiments, the composition or pharmaceutical formulation is administered via a viral vector. In some embodiments, the composition or pharmaceutical formulation is administered via lipid nanoparticles.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the methods and materials described herein will be apparent from the following detailed description and figures, and from the claims.

### BRIEF DESCRIPTION OF DRAWINGS

FIG. 1 shows the editing efficiency (Edit %) of 15 different gRNAs in HEK293 cells electroporated with RNP (ribonucleoprotein) comprising spCas9 protein and synthetic sgRNAs.

FIG. 2 shows the editing efficiency (Edit %) of 59 different gRNAs in HepG2 cells electroporated with RNP (ribonucleoprotein) comprising spCas9 protein and synthetic sgRNAs.

FIG. 3 is a plot of editing efficiency (Edit %) of sgRNAs targeting human TTR which were delivered to primary human hepatocytes from different donors using RNAi max. Guide sequences are ranked ordered based on highest % edit. The plot of FIG. 3 corresponds to the data presented in Table 7.

FIG. 4 is a is a plot of editing efficiency (% Edit) of sgRNAs targeting human TTR which were delivered to primary human hepatocytes from different donors using RNAi max. Guide sequences are ranked ordered based on highest % edit. The plot of FIG. 4 corresponds to the data presented in Table 8.

FIG. 5 is a plot of the EC50 and maximum editing of the tested human specific TTR sgRNAs with Spy Cas9 protein on HepG2 as dose response curves. The plot of FIG. 5 corresponds to the data presented in Table 9.

FIG. 6 is a plot of the EC50 and maximum editing of the tested human specific TTR sgRNAs with Spy Cas9 mRNA formulated in LNP on HepG2 as dose response curves. The plot of FIG. 6 corresponds to the data presented in Table 10.

FIG. 7 is a plot of the editing of the tested human specific TTR sgRNAs with Spy Cas9 mRNA formulated in LNP on primary human hepatocytes as concentration response curves.

FIG. 8 is a plot of percent reduction of TTR protein in primary human hepatocytes measured by Western blot after transfection with sgRNAs disclosed herein. Beta-actin was used as a loading control.

FIG. 9 is a plot of percent reduction of TTR protein in primary human hepatocytes measured by Western blot after transfection with sgRNAs disclosed herein. Beta-actin was used as a loading control.

FIG. 10 is a plot of percent reduction of TTR protein in primary human hepatocytes measured by ELISA after transfection with sgRNAs disclosed herein.

FIG. 11 is a plot of percent reduction of TTR protein in primary human hepatocytes measured by ELISA after transfection with sgRNAs disclosed herein.

FIG. 12 is a plot of editing efficiency for several sgRNAs disclosed herein after transfection of PHH cells with LNPs comprising Cas9 mRNA and sgRNA. FIG. 12 shows dose-response curves for several sgRNAs.

FIG. 13 is a plot of editing efficiency for several sgRNAs disclosed herein, showing off-target amp-seq at the maximum dose for several sgRNAs.

FIG. 14 is a plot of editing efficiency for several sgRNAs disclosed herein, showing off-target amp-seq at the maximum dose for several sgRNAs.

FIG. 15 is a plot of on-target editing efficiency of the combination of exonuclease fusion and sgRNA modification for several modified sgRNAs and exonuclease fusions disclosed herein.

FIG. 16 is a plot of off-target editing efficiency of the combination of exonuclease fusion and sgRNA modification for several modified sgRNAs and exonuclease fusions disclosed herein.

FIG. 17 is a plot of off-target editing efficiency of the combination of exonuclease fusion and sgRNA modification for several modified sgRNAs and exonuclease fusions disclosed herein.

FIG. 18 is a plot of serum TTR levels in mouse (μg/ml) after treatment with the LNP composition comprising sgRNA ART-001-g-066 and Cas9 mRNA in a 1:1 ratio by weight. The LNP had a N:P ratio of 3 and contained ALC0315, DSPC, Cholesterol, and PEG2k-DMG at a 50:10:38.5:1.5 molar ratio. Serum TTR was determined using ELISA.

FIG. 19 is a plot of serum TTR levels in mouse (μg/ml) and % TTR editing in liver tissue after treatment with the LNP composition comprising sgRNA ART-001-g-066 and Cas9 mRNA in a 1:1 ratio by weight. Dose levels were at 3, 1, or 0.3 mg/kg.

FIG. 20A is a plot of serum TTR levels in mouse (μg/ml) after treatment with the LNP composition comprising sgRNA ART-001-g-066 and Cas9 mRNA in a 1:1 ratio by weight.

FIG. 20B is a plot of serum TTR levels in mouse (μg/ml) after treatment with the LNP composition comprising sgRNA ART-001-g-066 and Cas9 mRNA in a 1:1 ratio by weight.

FIG. 21 is a plot of serum TTR levels in mouse (μg/ml) after treatment with several doses of the LNP composition comprising sgRNA ART-001-g-066 and Cas9 mRNA at a 1:1 or 1:2 weight ratio. CD-1 female mice were dosed i.v. at 0.1, 0.3, and 1 mg/kg.

FIG. 22 is a plot of serum TTR levels in mouse (μg/ml) after treatment with LNP comprising different amine lipids. LNPs were formulated with a guide RNA ART-001-g-067. CD-1 female mice were dosed i.v. at 0.3 mg/kg.

FIG. 23 is a plot of normalized human protein in serum (%) in humanized TTR V30M knock-in mice after treatment with several sgRNAs disclosed herein.

FIG. 24 is a plot of normalized human protein in serum (%) in humanized TTR V30M knock-in mice after treatment with several sgRNAs disclosed herein. Dosing levels were at 1 or 3 mg/kg.

FIG. 25A is a plot of percent TTR knockdown after treatment of TTR humanized mice with compositions comprising LNPs comprising several hybrid DNA-RNA sgRNAs disclosed herein. Dosing was at 0.1, 0.3, 0.6 mg/kg.

FIG. 25B is a plot of percent TTR knockdown after treatment of TTR humanized mice with compositions comprising LNPs comprising several hybrid DNA-RNA sgRNAs disclosed herein. Dosing was at 0.1, 0.3, 0.6 mg/kg.

### DETAILED DESCRIPTION

This disclosure features compositions and methods for editing the human transthyretin (TTR) gene. The compositions and methods described herein are for treating subjects having amyloidosis associated with transthyretin (ATTR).

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

### Definitions

Unless stated otherwise, the following terms and phrases as used herein are intended to have the following meanings:

As used herein, the term “nucleic acid” refers to a multimeric compound that has nucleosides or nucleoside analogs which have nitrogenous heterocyclic bases or base analogs linked together along a backbone, including conventional RNA, DNA, mixed RNA-DNA, and polymers that are analogs thereof. The terms “nucleic acid,” “polynucleotide,” “nucleotide,” “nucleotide sequence,” and “oligonucleotide” are used interchangeably. They refer to a polymeric form of nucleotides of any length, either deoxyribonucleotides or ribonucleotides, or analogs thereof. The following are non-limiting examples of nucleic acids: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A nucleic acid backbone can be made up of a variety of linkages, including one or more of sugar-phosphodiester linkages, peptide-nucleic acid bonds (“peptide nucleic acids” or PNAs such as those described in International Patent Publication No. WO1995032305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O6-methylguanine, 4-thio-pyrimidines, 4-amino-pyrimidines, 4-dimethylhydrazine-pyrimidines, and 04-alkyl-pyrimidines; (See e.g., U.S. Pat. No. 5,378,825 and International Patent Publication No. WO1993013121). For general discussion, see The Biochemistry of the Nucleic Acids 5-36, Adams et al., ed., 11th ed., 1992). Nucleic acids can include one or more “abasic” residues where the backbone includes no nitrogenous base for position(s) of the polymer (See e.g., U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43 (42): 13233-41). RNA and DNA have different sugar moieties and can differ by the presence of uracil or analogs thereof in RNA and thymine or analogs thereof in DNA.

As used herein, the term “guide RNA” refer to the combination of a CRISPR RNA (crRNA) and a tracr RNA (trRNA). “Guide RNA” can be used interchangeably with “gRNA,” or “guide”. The crRNA and trRNA may be associated as a single RNA molecule (single guide RNA, sgRNA) or in two separate RNA molecules (dual guide RNA, dgRNA). “Guide RNA” or “gRNA” can refer to each type, i.e., sgRNA or dgRNA. The trRNA may be a naturally-occurring sequence, or a trRNA sequence can have modifications or variations compared to naturally-occurring sequences. Guide RNAs can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be about 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the guide sequence and the targeting sequence may be 100% complementary or identical in sequence to one another. In other embodiments, the guide sequence and the targeting sequence may contain at least one mismatch. For example, the guide sequence and the targeting sequence may contain 1, 2, 3, or 4 mismatches, where the total length of the targeting sequence is at least 17, 18, 19, 20 or more base pairs. In some embodiments, the guide sequence and the targeting sequence may contain 1-4 mismatches where the guide sequence comprises at least 17, 18, 19, 20 or more nucleotides. In some embodiments, the guide sequence and the targeting sequence may contain 1, 2, 3, or 4 mismatches where the guide sequence comprises at least 20 nucleotides.

In some embodiments, the guide RNA comprises a crRNA that has a guide sequence (e.g., a guide sequence from Table 3) and further includes a nucleotide sequence GUU UUA GAG CUA UGC UGU UUU G (SEQ ID NO: 256), wherein SEQ ID NO: 256 follows the guide sequence at its 3′ end. In some embodiments, the crRNA is any crRNA selected from the nucleotide sequences set forth in SEQ ID NOs: 86-166 or SEQ ID NOs 308-340. In some embodiments, the guide RNA comprises any one of the crRNA nucleotide sequences set forth in SEQ ID NOs: 86-166 or SEQ ID NOs 308-340.

In some embodiments, the guide RNA comprises a crRNA and further includes a tracrRNA (trRNA) sequence comprising the nucleotide sequence set forth in SEQ ID NO: 257 or a portion thereof. AAC AGC AUA GCA AGU UAA AAU AAG GCU AGU CCG UUA UCA ACU UGA AAA AGU GGC ACC GAG UCG GUG CUU UUU UU (SEQ ID NO: 257).

In some embodiments, the guide RNA comprises additional nucleotides to form a sgRNA, e.g., with the following exemplary nucleotide sequence following the 3′ end of the guide sequence: GUU UUA GAG CUA GAA AUA GCA AGU UAA AAU AAG GCU AGU CCG UUA UCA ACU UGA AAA AGU GGC ACC GAG UCG GUG CUU UU (SEQ ID NO: 258) in the 5′ to 3′ orientation. In some embodiments, the sgRNA is any sgRNA selected from the nucleotide sequences set forth in SEQ ID NOs: 171-251 or SEQ ID NOs 341-373. In some embodiments, the gRNA comprises any one of the nucleotide sequences set forth in SEQ ID NOs: 171-251 or SEQ ID NOs 341-373. In some embodiments, the gRNA consists of any one of the nucleotide sequences set forth in SEQ ID NOs: 171-251 or SEQ ID NOs 341-373.

In some embodiments, the guide RNA comprises a portion of SEQ ID NO: 256 covalently linked to a trRNA. For instance, the guide RNA comprises a guide sequence (e.g., a guide sequence from Table 3) linked to GUUUUAGAGCUA (SEQ ID NO: 259) further linked to a trRNA (SEQ ID NO: 257 or a portion thereof). For instance, the guide RNA comprises a guide sequence (e.g., a guide sequence from Table 3) linked to GUU UUA GAG CUA (SEQ ID NO: 259) further linked to the nucleotide sequence AUA GCA AGU UAA AAU AAG GCU AGU CCG UUA UCA ACU UGA AAA AGU GGC ACC GAG UCG GUG CUU UU (SEQ ID NO: 260).

Targeting sequences for Cas proteins include both the positive and negative strands of genomic DNA (i.e., the sequence given and the sequence's reverse complement), since the nucleic acid substrate for a Cas protein is double stranded. Accordingly, where a guide sequence is said to be “complementary to a target sequence”, it is to be understood that the guide sequence may direct a guide RNA to bind to the reverse complement of a target sequence. Thus, in some embodiments where the guide sequence binds the reverse complement of a target sequence, the guide sequence is identical to certain nucleotides of the target sequence (e.g., the target sequence not including the protospacer adjacent motif (PAM)) except for the substitution of U for Tin the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents (such as those described in International Patent Application Nos. WO2020198697, incorporated herein in its entirety) include Cas nickases and inactivated forms thereof, such as dCas DNA binding agents”.

As used herein, the term “Cas” refers to any Cas protein that is operable for gene editing using a guide molecule. “Cas nuclease” also encompasses Cas nickases, and endonuclease-deficient or dead Cas (dCas) DNA binding agents. Cas nickases and dCas DNA binding agents can include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas nickases (e.g., H840A, D10A, or N863A variants), which further have RNA-guided DNA nickase activity, and Class 2 dCas DNA binding agents, in which nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g, K810A, K1003A, R1060A variants), and eSPCas9 (1. 1) (e.g., K848A, K1003A, R1060A variants) proteins and modifications thereof. Cpf1 protein, Zetsche et al, Cell, 163:1-13 (2015), is homologous to Cas9, and contains a RuvC-like nuclease domain. Cpf1 sequences of Zetsche are incorporated by reference in their entirety. See, e.g., Zetsche, Tables SI and S3. “Cas9” encompasses Spy Cas9, the variants of Cas9 listed herein, and equivalents thereof. See, e.g., Makarova et al, Nat Rev Microbiol, 13 (11): 722-36 (2015); Shmakov et al., Molecular Cell, 60:385-397 (2015).

dCas DNA binding agents can be used in CRISPR interference (CRISPRi) as well as CRISPR activation (CRISPRa). In CRISPRi, dCas9 binds to its DNA target but does not cleave it. Without being bound by theory, it is believed that the binding of Cas9 alone will prevent the cell's transcription machinery from accessing the promoter, hence inhibiting the gene expression. On the other hand, dCas9's ability to bind target DNA can be exploited for activation, i.e., CRISPRa. A transcriptional activator is fused to dCas9, which can activate gene expression without changing DNA sequence. In some embodiments, the dCas DNA binding agent is fused to a repressor, such as a Krüppel-associated box (KRAB).

“Modified uridine” is used herein to refer to a nucleoside with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton, e.g., Nl-methyl pseudouridine. In some embodiments, a modified uridine is any of a substituted uridine, pseudouridine, or a substituted pseudouridine

As used herein, a first sequence is considered to “comprise a sequence that is at least X % identical to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) bind the same complement nucleotide(s) (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity>50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, the term “mRNA” refers to a polynucleotide that is RNA or modified RNA and includes an open reading frame that can be translated into a polypeptide (i.e., can serve as a substrate for translation by a ribosome and amino-acylated tRNAs). mRNA can include a phosphate-sugar backbone having ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof.

As used herein, the term “TTR” refers to transthyretin, which is the expressed product of a TTR gene. The human wild-type TTR sequence is available at NCBI Gene ID: 7276; Ensembl: Ensembl: ENSG00000118271. Transthyretin is a 56-kDa nonglycosylated protein composed of four identical 127-amino acid subunits. The single-copy transthyretin gene is located on chromosome 18 and composed of four exons spanning 7.3 kilobase pairs. The 5′-flanking region has a highly homologous DNA sequence across species, suggesting its crucial role in the regulation of transthyretin gene expression. Transthyretin is mostly of liver origin, but it is also synthesized in pancreatic islet cells, retina, and epithelial cells of choroid plexus in both rats and humans. As used herein, “mutant TTR” refers to a gene product of TTR (i.e., the TTR protein) having a change in the amino acid sequence of TTR compared to the wildtype amino acid sequence of TTR. Mutants forms of TTR associated with ATTR, e.g., in humans, include V30M, V30A, V30G, V30L, T60A, V122I, V122A, or V122(−).

As used herein, “amyloid” refers to abnormal aggregates of proteins or peptides that are normally soluble. Amyloids are insoluble, and amyloids can create proteinaceous deposits in organs and tissues. Proteins or peptides in amyloids may be misfolded into a form that allows many copies of the protein to stick together to form fibrils. While some forms of amyloid may have normal functions in the human body, “amyloids” as used herein refers to abnormal or pathologic aggregates of protein. Amyloids may comprise a single protein or peptide, such as TTR, or they may comprise multiple proteins or peptides, such as TTR and additional proteins.

As used herein, “amyloid fibrils” refers to insoluble fibers of amyloid that are resistant to degradation. Amyloid fibrils can produce symptoms based on the specific protein or peptide and the tissue and cell type in which it has aggregated.

As used herein, “amyloidosis” refers to a disease characterized by symptoms caused by deposition of amyloid or amyloid fibrils. Amyloidosis can affect numerous organs including the heart, kidney, liver, spleen, nervous system, and digestive track.

As used herein, “ATTR,” “ATTR amyloidosis,” or “amyloidosis associated with TTR” refers to amyloidosis that occurs due to the deposition of TTR amyloid fibrils.

As used herein, “familial amyloid cardiomyopathy” or “FAC” refers to a hereditary transthyretin amyloidosis (ATTR) characterized primarily by restrictive cardiomyopathy. Congestive heart failure is common in FAC. Average age of onset is approximately 60-70 years of age, with an estimated life expectancy of 4-5 years after diagnosis.

As used herein, “familial amyloid polyneuropathy” or “FAP” refers to a hereditary transthyretin amyloidosis (ATTR) characterized primarily by sensorimotor neuropathy. Autonomic neuropathy is common in FAP. While neuropathy is a primary feature, symptoms of FAP may also include cachexia, renal failure, and cardiac disease. Average age of onset of FAP is approximately 30-50 years of age, with an estimated life expectancy of 5-15 after diagnosis.

As used herein, “wild-type ATTR” and “ATTRwt” refer to ATTR not associated with a pathological TTR mutation such as T60A, V30M, V30A, V30G, V30L, V122I, V122A, or V122(−). ATTRwt has also been referred to as senile systemic amyloidosis. Onset typically occurs in men aged 60 or higher with the most common symptoms being congestive heart failure and abnormal heart rhythm such as atrial fibrillation. Additional symptoms include consequences of poor heart function such as shortness of breath, fatigue, dizziness, swelling (especially in the legs), nausea, angina, disrupted sleep, and weight loss. A history of carpal tunnel syndrome indicates increased risk for ATTRwt and may in some cases be indicative of early-stage disease. ATTRwt generally leads to decreasing heart function over time but can have a better prognosis than hereditary ATTR because wild-type TTR deposits accumulate more slowly. Existing treatments are similar to other forms of ATTR (other than liver transplantation) and are generally directed to supporting or improving heart function, ranging from diuretics and limited fluid and salt intake to anticoagulants, and in severe cases, heart transplants. Nonetheless, like FAC, ATTRwt can result in death from heart failure, sometimes within 3-5 years of diagnosis.

As used herein, “hereditary ATTR” refers to ATTR that is associated with a mutation in the sequence of the TTR gene. Known mutations in the TTR gene associated with ATTR include those resulting in TTR with substitutions of T60A, V30M, V30A, V30G, V30L, V122I, V122A, or V122(−). Other mutations include V28S, F33V, F33L, K35N, K35T, A36P, D38V, E42G, G47R, G47V, G47E, T49A, S50R, G53E, E54K, E54G, E54Q, T59K, E61K, Y69H, S77Y, S77F, G83R, A97S, or Y114C.

As used herein, the term “pathological mutation” refers to a mutation that renders a gene product, such as TTR, more likely to cause, promote, contribute to, or fail to inhibit the development of a disease, such as ATTR.

As used herein, “indels” refer to insertion/deletion mutations consisting of a number of nucleotides that are either inserted or deleted at the site of double-stranded breaks (DSBs) in a target nucleic acid.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest before and after knockdown. Methods for measuring knockdown of mRNA are known in the art, and include sequencing of mRNA isolated from a tissue or cell population of interest. In some embodiments, “knockdown” may refer to some loss of expression of a particular gene product, for example, a decrease in the amount of mRNA transcribed or a decrease in the amount of protein expressed or secreted by a population of cells (including in vivo populations such as those found in tissues).

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” or “treating” refers to an improvement, alleviation, or amelioration of at least one symptom of a disclosed condition upon administration or application of a therapeutic for the condition. The term includes inhibiting the condition or disease, arresting its development, relieving one or more symptoms of the condition or disease, curing the condition or disease, or preventing reoccurrence of one or more symptoms of the condition or disease. In the context of this disclosure, treatment of ATTR may comprise alleviating symptoms of ATTR. A treatment with the compositions of this disclosure is said to have “treated” the condition if the treatment results in a reduction in the pathology of the condition (e.g., amyloid deposition).

As used herein, the term “lipid nanoparticle” (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. See also, e.g., WO2015006747, WO2016118724, WO2021026358, WO2017173054 and WO2019067992, the contents of which are incorporated herein by reference in their entireties. Any LNP known to those of skill in the art to be capable of delivering nucleotides to subjects may be utilized with the guide RNAs and the nucleic acid encoding an RNA-guided DNA binding agent described herein.

As used herein, the term “pharmaceutically acceptable” means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects.

As used herein, “infusion” refers to an active administration of one or more agents with an infusion time of, for example, between approximately 30 minutes and 12 hours. In some embodiments, the one or more agents comprise an LNP, e.g., having an mRNA encoding an RNA-guided DNA binding agent (such as Cas9) described herein and a gRNA described herein.

The term “about” or “approximately” means an acceptable error for a particular value as determined by one of ordinary skill in the art, which depends in part on how the value is measured or determined.

Numeric ranges are inclusive of the numbers defining the range. Measured and measureable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

### Compositions and Methods Targeting the TTR Gene

Disclosed herein are compositions for use in methods targeting the TTR gene. The methods disclosed herein induce a double-stranded break (DSB) within the TTR gene in a subject, modify the TTR gene in a cell or subject, treat amyloidosis associated with TTR (ATTR) in a subject, reduce TTR monomeric, dimeric, and/or tetrameric serum concentration in a subject, and/or reduce or prevent the accumulation of amyloids or amyloid fibrils in a subject. In some embodiments, the disclosed compositions and methods inhibit the transcription and translation of TTR, thereby preventing the accumulation of TTR in tissues. In general, the disclosed compositions comprise a guide RNA targeting TTR (itself or in a vector), and an RNA-guided DNA binding agent, or a nucleic acid encoding an RNA-guided DNA binding agent (e.g., a CRISPR/Cas system). The subjects treated with such methods and compositions may have wild-type or non-wild type TTR gene sequences, such as, for example, subjects with ATTR, which may be ATTR wt or a hereditary or familial form of ATTR. In some embodiments, the composition is administered by infusion for 0.5-6 hours. In some embodiments, the composition is administered by subcutaneous injection. In some embodiments, the composition is administered by intrathecal injection.

A. Guide RNA (gRNAs)

The guide RNA used in the disclosed methods and compositions comprises a guide sequence targeting the TTR gene. Exemplary guide sequences targeting the TTR gene are shown in Table 3 at SEQ ID NOs: 1-85 and SEQ ID NOs: 275-307. Guide sequences useful in the guide RNA compositions and methods described herein are shown in Table 3 and throughout the application.

Each of the guide sequences in Table 3 may further comprise additional nucleotides to form a crRNA, e.g., with the following exemplary nucleotide sequence following the guide sequence at its 3′ end: GUU UUA GAG CUA UGC UGU UUU G (SEQ ID NO: 256). In the case of a sgRNA, the guide sequences of Table 3 may further comprise additional nucleotides to form a sgRNA, e.g., with the following exemplary nucleotide sequence following the 3′ end of the guide sequence, wherein the sgRNA has a custom-designed short crRNA component followed by the trRNA component: GUU UUA GAG CUA GAA AUA GCA AGU UAA AAU AAG GCU AGU CCG UUA UCA ACU UGA AAA AGU GGC ACC GAG UCG GUG CUU UU (SEQ ID NO: 258) in the 5′ to 3′ orientation.

SEQ ID NO: 258 is attached to the 3′ end of the guide sequence in the in the 5′ to 3′ orientation. sgRNA sequences useful in the compositions and methods of this disclosure are described in Table 4.

In some embodiments, the sgRNA is modified. In some embodiments, the sgRNA comprises the modification pattern shown below in SEQ ID NO: 261, where Nis any natural or non-natural nucleotide, and where the totality of the N's comprise a guide sequence as described herein and the modified sgRNA comprises the following sequence: mN*mN*mN*NNNNNNNNNNNNNNNNNGUUUUAGAmGmCmUmAmGmAmAmA mUmAmGmCAAGUUAAAAUAAGGCUAGUCCGUUAUCAmAmCmUmUm GmAmAmAmAmAmGmUmGmGmCmAmCmCmGmAmGmUmCmGmGmUmGmCm U*mU*mU*mU (SEQ ID NO: 261), where “N” may be any natural or non-natural nucleotide; *=PS linkage; ‘m’=2′-O-Me nucleotide. The modifications remain as shown in SEQ ID NO: 261 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

In some embodiments, the gRNA sequence has the modification pattern described in WO2016164356 and WO2016089433, each of which is incorporated herein in its entirety.

In some embodiments, the gRNA comprises a guide sequence that direct an RNA-guided DNA binding agent, which can be a nuclease (e.g., a Cas nuclease such as Cas9), to a target DNA sequence in TTR. The gRNA includes a crRNA having a guide sequence shown in Table 3. The gRNA includes a guide sequence having at least 15, 16, 17, 18, 19, or 20 contiguous nucleotides of any one of the guide sequences of SEQ ID NOs: 1-47 shown in Table 3, or at least 18, 19, or 20 contiguous nucleotides of any one of the guide sequences of SEQ ID NOs: 48-81 shown in Table 3 or at least 18, 19, or 20 contiguous nucleotides of any one of the guide sequences of SEQ ID NOs: 275-307 shown in Table 3. In some embodiments, the gRNA comprises a guide sequence having a sequence with about 75%, 80%, 85%, 90%, 91%, 92%, 93%, 94%, 95%, 96%, 97%, 98%, or 99% identity to at least 16, 17, 18, 19, or 20 contiguous nucleotides of any one of the guide sequences of SEQ ID NOs: 1-47 shown in Table 3, or a sequence with about 75%, 80%, 85%, 90%, 91%, 92%, 93%, 94%, 95%, 96%, 97%, 98%, or 99% identity to at least 18, 19, or 20 contiguous nucleotides of any one of the guide sequences of SEQ ID NOs: 48-81, or a sequence with about 75%, 80%, 85%, 90%, 91%, 92%, 93%, 94%, 95%, 96%, 97%, 98%, or 99% identity to at least 18, 19, or 20 contiguous nucleotides of any one of the guide sequences of SEQ ID NOs: 275-307. The gRNA may further comprise a tracr RNA (trRNA). In each composition and method embodiment described herein, the crRNA and trRNA may be associated as a single RNA (sgRNA), or may be on separate RNAs (dgRNA). In the context of sgRNAs, the crRNA and trRNA components may be covalently linked, e.g., via a phosphodiester bond or other covalent bond.

In each of the composition, use, and method embodiments described herein, the guide RNA may comprise two RNA molecules as a “dual guide RNA” or “dgRNA”. The dgRNA comprises a first RNA molecule comprising a crRNA having, e.g., a guide sequence shown in Table 3, and a second RNA molecule having a trRNA. The first and second RNA molecules may not be covalently linked, but may form a RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) having a guide sequence shown in Table 3 covalently linked to a trRNA. The sgRNA may comprise at least 15, 16, 17, 18, 19, or 20 contiguous nucleotides of any one of the guide sequences of SEQ ID NOs: 1-47 shown in Table 3, or at least 18, 19, or 20 contiguous nucleotides of any one of the guide sequences of SEQ ID NOs: 48-81 shown in Table 3, or at least 18, 19, or 20 contiguous nucleotides of any one of the guide sequences of SEQ ID NOs: 275-307 shown in Table 3. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures. In some embodiments, the composition comprises a gRNA that comprises a guide sequence that is at least 90%, 91%, 92%, 93%, 94%, 95%, 96%, 97%, 98%, or 99% identity to at least 16, 17, 18, 19, or 20 contiguous nucleotides of any one of the guide sequences of SEQ ID NOs: 1-47 shown in Table 3, or a sequence with about 75%, 80%, 85%, 90%, 90%, 91%, 92%, 93%, 94%, 95%, 96%, 97%, 98%, or 99% identity to at least 18, 19, or 20 contiguous nucleotides of any one of the guide sequences of SEQ ID NOs: 48-81, or a sequence with about 75%, 80%, 85%, 90%, 90%, 91%, 92%, 93%, 94%, 95%, 96%, 97%, 98%, or 99% identity to at least 18, 19, or 20 contiguous nucleotides of any one of the guide sequences of SEQ ID NOs: 275-307.

In some embodiments, the composition includes a guide RNA having a guide sequence selected from SEQ ID NOs: 1-81 or SEQ ID NOs: 275-307. The guide RNA having a guide sequence selected from SEQ ID NOs: 1-81 or SEQ ID NOs: 275-307 may be a chemically modified sgRNA, such as an end modified RNA. The guide RNA having a guide sequence selected from SEQ ID NOs: 1-81 or SEQ ID NOs: 275-307 may be dgRNA, such as a chemically modified dgRNA.

In other embodiments, the composition comprises at least one, e.g., at least two gRNAs having guide sequences selected from any two or more of the guide sequences of SEQ ID NOs: 1-81 or SEQ ID NOs: 275-307. In some embodiments, the composition comprises at least two gRNAs that each comprise a guide sequence at least 90%, 90%, 91%, 92%, 93%, 94%, 95%, 96%, 97%, 98%, or 99% identical to any of the nucleic acids of SEQ ID NOs: 1-81 or SEQ ID NOs: 275-307.

In some embodiments, the gRNA is a sgRNA having any one of SEQ ID NOs. 171-251 or SEQ ID NOs. 341-373. In some embodiments, the gRNA is a sgRNA having any one of SEQ ID NOs. 171-251 or SEQ ID NOs. 341-373, but without the modifications described in this disclosure (i.e., unmodified SEQ ID NOs. 171-251 or SEQ ID NOs. 341-373). In some embodiments, the gRNA is a sgRNA having any one of SEQ ID NOs. 171-251 or SEQ ID NOs. 341-373, but with at least one chemical modification. In some embodiments, the chemically modified SEQ ID NOs. 171-251 or SEQ ID NOs. 341-373 have 5′ and/or 3′ end modifications. In some embodiments, the gRNA is a sgRNA having any one of SEQ ID NOs. 171-251 or SEQ ID NOs. 341-373, but with the modification pattern shown in SEQ ID NO: 261.

The guide RNAs provided herein can be useful for recognizing (e.g., hybridizing to) a target sequence in the TTR gene. For example, the TTR target sequence may be recognized and cleaved by a provided Cas nuclease having a guide RNA. Thus, an RNA-guided DNA binding agent, such as a Cas nuclease, may be directed by a guide RNA to a target sequence of the TTR gene, where the guide sequence of the guide RNA hybridizes with the target sequence and the RNA-guided DNA binding agent, such as a Cas nuclease, cleaves the target sequence.

In some embodiments, the selection of the one or more guide RNAs is determined based on target sequences within the TTR gene. For example, the one or more guide RNAs is based on target sequences within any one of Exons 1˜4 or the 5′UTR region of the TTR gene.

Without being bound by any particular theory, mutations (e.g., frameshift mutations resulting from indels occurring as a result of a nuclease-mediated DSB) in certain regions of the gene may be less tolerable than mutations in other regions of the gene, thus, the location of a DSB is an important factor in the amount or type of protein knockdown that may result. In some embodiments, a gRNA complementary or having complementarity to a target sequence within TTR is used to direct the RNA-guided DNA binding agent to a particular location in the TTR gene. In some embodiments, gRNAs are designed to have guide sequences that are complementary or have complementarity to target sequences in exon 1, exon 2, exon 3, or exon 4 of TTR. In some embodiments, a frameshift or nonsense mutation is induced in the TTR gene of about 10%, about 15%, about 20%, about 25%, about 30% of cells to about 35% of the cells.

B. Modifications of gRNAs

In some embodiments, the gRNA is chemically modified. A gRNA having one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues, including T. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs having nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxy methyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e., at sugar modification. For example, the 2′ hydroxyl group (OH) can be modified, e.g., replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH2CH2O)n CH2CH2OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, O(CH2) n-amino, (wherein amino can be, e.g., NH2; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxy ethyl group (MOE), (OCH2CH2OCH3, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NEE; alkylamino, dialkylamino, heterocyclyl, arylamino, diarylamino, heteroarylamino, diheteroarylamino, or amino acid); NH(CH2CH2NH)nCH2CH2-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with, e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g, arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), thymidine (T) and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments having an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a guide RNA molecule are deoxynucleotides.

In some embodiments, a gRNA can have one or more modifications. In some embodiments, the modification includes a 2′-O-methyl (2′-O-Me) modified nucleotide. In some embodiments, the modification includes a phosphorothioate (PS) bond between nucleotides.

The terms “mA,” “mC,” “mU,” or “mG” may be used to denote a nucleotide that has been modified with 2′-O-Me.

In some embodiments, the guide RNA includes a sgRNA having a guide sequence selected from SEQ ID NOs: 1-81 or SEQ ID NOs: 275-307 and the nucleotides of SEQ ID NO: 258, wherein the nucleotides of SEQ ID NO: 258 are on the 3′ end of the guide sequence, and wherein the guide sequence may be modified as shown in SEQ ID NO: 261.

Further examples of gRNA modifications are shown in e.g., WO2020198697, WO2016164356, and WO2016089433, incorporated by reference herein in its entirety.

### C. PAM Sequences

The PAM, also known as the protospacer adjacent motif, follows the target DNA sequence that is essential for cleavage by Cas nuclease. The PAM is about 2-8 nucleotides downstream of the DNA sequence targeted by the guide RNA and the Cas cuts 3-4 nucleotides upstream of it. PAM sequences are exemplified below in Tables 1-2. A PAM in the context of this disclosure can be any one of the sequences in Tables 1-2 or any other sequence known in the art.

TABLE 1
PAM of synthetic spCas9 variants

| SpCas9 variant | Mutations (relative to SpCas9) | PAM sequence |
|---|---|---|
| D1135E variant | D1135E | NGG (SEQ ID NO: 262) |
| VQR variant | D1135V, R1335Q and T1337R | NGAN (SEQ ID NO: 263) or |
| | | NGNG (SEQ ID NO: 264) |
| EQR variant | D1135E, R1335Q and T1337R | NGAG (SEQ ID NO: 265) |
| VRER variant | D1135V, G1218R, R1335E and T1337R | NGCG (SEQ ID NO: 266) |

N is A, G, C or T.

TABLE 2
PAM of different Cas9 species

| Cas9 species | PAM sequence |
|---|---|
| Streptococcus pyogenes (Sp) | NGG (SEQ ID NO: 262) |
| Staphylococcus aureus (Sa) | NGRRN (SEQ ID NO: 267) |
| Neisseria meningitidis (Nm or Nme) | NNNNGATT (SEQ ID NO: 268) |
| Campylobacter jejuni (Cj) | NNNNRYAC (SEQ ID NO: 269) |
| Streptococcus thermophilus (St) | NNAGAAW (SEQ ID NO: 270) |
| Treponema denticola (Td) | NAAAAC (SEQ ID NO: 271) |

N is A, G, C or T.

### D. RNA-Guided DNA Binding Agent

Any nucleic acid having an open reading frame encoding an RNA-guided DNA binding agent, e.g. a Cas9 nuclease such as an S. pyogenes Cas9, may be combined in a composition or method with any of the gRNAs disclosed herein. In some embodiments, the nucleic acid having an open reading frame encoding an RNA-guided DNA binding agent is an mRNA. In some embodiments, the RNA-guided DNA binding agent is administered in its amino acid form, i.e., as a protein. In some embodiments, the nucleic acid encoding the RNA-guided DNA binding agent is part of a vector described herein. The nucleic acid encoding the RNA-guided DNA binding agent may have any of the characteristics described in WO2020198697, incorporated by reference herein in its entirety.

In some embodiments, the RNA-guided DNA binding agent for use in the compositions and methods described herein the RNA-guided DNA-binding agent is a Class 2 Cas nuclease. In some embodiments, the RNA-guided DNA-binding agent has double-strand endonuclease activity. In some embodiments, the RNA-guided DNA-binding agent comprises a Cas nuclease, such as a Class 2 Cas nuclease (which may be, e.g., a Cas nuclease of Type II, V, or VI). Class 2 Cas nucleases include, for example, Cas9, Cpf1, C2c1, C2c2, and C2c3 proteins and modifications thereof.

Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas 10, Csm1, or Cmr2 subunit thereof; and a Cascade complex of a type I CRISPR system, or the Cas3 subunit thereof. In some embodiments, the Cas nuclease may be from a Type-IIA, Type-11B, or Type-IIC system For discussion of various CRISPR systems and Cas nucleases see, e.g., Makarova et al., Nat. Rev. Microbiol. 9:467-477 (2011); Makarova et al., Nat. Rev. Microbiol, 13:722-36 (2015); Shmakov et al., Molecular Cell, 60:385-397 (2015). In some embodiments, the RNA-guided DNA binding agent is a Cas nickase, e.g. a Cas9 nickase. In some embodiments, the RNA-guided DNA binding agent is an S. pyogenes Cas9 nuclease.

Non-limiting exemplary species that the RNA-guided DNA binding agent (e.g., the Cas nuclease) can be derived from include but are not limited to Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter Jejuni, Pasteurella multocida, Fibrobacter succinogene, Rhodospirillum rubrum, Nocardiopsis dassonvillei, Streptomyces pristinaespiralis, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter zari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In some embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In some embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In certain embodiments, the Cas nuclease can cleave one or both strands of dsDNA. In some embodiments, the Cas nuclease can cleave a single strand of DNA. In some embodiments, the Cas nuclease may not have DNA nickase activity. An exemplary Cas9 amino acid sequence is provided as SEQ ID NO: 272.

(SEQ ID NO: 272)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGDGGGS
PKKKRKV

An exemplary Cas9 mRNA ORF sequence, which includes start and stop codons, is provided as SEQ ID NO: 273.

(SEQ ID NO: 273)
AUGGACAAGAAGUACAGCAUCGGACUGGACAUCGGAACAAACAGCGUCG
GAUGGGCAGUCAUCACAGACGAAUACAAGGUCCCGAGCAAGAAGUUCAA
GGUCCUGGGAAACACAGACAGACACAGCAUCAAGAAGAACCUGAUCGGA
GCACUGCUGUUCGACAGCGGAGAAACAGCAGAAGCAACAAGACUGAAGA
GAACAGCAAGAAGAAGAUACACAAGAAGAAAGAACAGAAUCUGCUACCU
GCAGGAAAUCUUCAGCAACGAAAUGGCAAAGGUCGACGACAGCUUCUUC
CACAGACUGGAAGAAAGCUUCCUGGUCGAAGAAGACAAGAAGCACGAAA
GACACCCGAUCUUCGGAAACAUCGUCGACGAAGUCGCAUACCACGAAAA
GUACCCGACAAUCUACCACCUGAGAAAGAAGCUGGUCGACAGCACAGAC
AAGGCAGACCUGAGACUGAUCUACCUGGCACUGGCACACAUGAUCAAGU
UCAGAGGACACUUCCUGAUCGAAGGAGACCUGAACCCGGACAACAGCGA
CGUCGACAAGCUGUUCAUCCAGCUGGUCCAGACAUACAACCAGCUGUUC
GAAGAAAACCCGAUCAACGCAAGCGGAGUCGACGCAAAGGCAAUCCUGA
GCGCAAGACUGAGCAAGAGCAGAAGACUGGAAAACCUGAUCGCACAGCU
GCCGGGAGAAAAGAAGAACGGACUGUUCGGAAACCUGAUCGCACUGAGC
CUGGGACUGACACCGAACUUCAAGAGCAACUUCGACCUGGCAGAAGACG
CAAAGCUGCAGCUGAGCAAGGACACAUACGACGACGACCUGGACAACCU
GCUGGCACAGAUCGGAGACCAGUACGCAGACCUGUUCCUGGCAGCAAAG
AACCUGAGCGACGCAAUCCUGCUGAGCGACAUCCUGAGAGUCAACACAG
AAAUCACAAAGGCACCGCUGAGCGCAAGCAUGAUCAAGAGAUACGACGA
ACACCACCAGGACCUGACACUGCUGAAGGCACUGGUCAGACAGCAGCUG
CCGGAAAAGUACAAGGAAAUCUUCUUCGACCAGAGCAAGAACGGAUACG
CAGGAUACAUCGACGGAGGAGCAAGCCAGGAAGAAUUCUACAAGUUCAU
CAAGCCGAUCCUGGAAAAGAUGGACGGAACAGAAGAACUGCUGGUCAAG
CUGAACAGAGAAGACCUGCUGAGAAAGCAGAGAACAUUCGACAACGGAA
GCAUCCCGCACCAGAUCCACCUGGGAGAACUGCACGCAAUCCUGAGAAG
ACAGGAAGACUUCUACCCGUUCCUGAAGGACAACAGAGAAAAGAUCGAA
AAGAUCCUGACAUUCAGAAUCCCGUACUACGUCGGACCGCUGGCAAGAG
GAAACAGCAGAUUCGCAUGGAUGACAAGAAAGAGCGAAGAAACAAUCAC
ACCGUGGAACUUCGAAGAAGUCGUCGACAAGGGAGCAAGCGCACAGAGC
UUCAUCGAAAGAAUGACAAACUUCGACAAGAACCUGCCGAACGAAAAGG
UCCUGCCGAAGCACAGCCUGCUGUACGAAUACUUCACAGUCUACAACGA
ACUGACAAAGGUCAAGUACGUCACAGAAGGAAUGAGAAAGCCGGCAUUC
CUGAGCGGAGAACAGAAGAAGGCAAUCGUCGACCUGCUGUUCAAGACAA
ACAGAAAGGUCACAGUCAAGCAGCUGAAGGAAGACUACUUCAAGAAGAU
CGAAUGCUUCGACAGCGUCGAAAUCAGCGGAGUCGAAGACAGAUUCAAC
GCAAGCCUGGGAACAUACCACGACCUGCUGAAGAUCAUCAAGGACAAGG
ACUUCCUGGACAACGAAGAAAACGAAGACAUCCUGGAAGACAUCGUCCU
GACACUGACACUGUUCGAAGACAGAGAAAUGAUCGAAGAAAGACUGAAG
ACAUACGCACACCUGUUCGACGACAAGGUCAUGAAGCAGCUGAAGAGAA
GAAGAUACACAGGAUGGGGAAGACUGAGCAGAAAGCUGAUCAACGGAAU
CAGAGACAAGCAGAGCGGAAAGACAAUCCUGGACUUCCUGAAGAGCGAC
GGAUUCGCAAACAGAAACUUCAUGCAGCUGAUCCACGACGACAGCCUGA
CAUUCAAGGAAGACAUCCAGAAGGCACAGGUCAGCGGACAGGGAGACAG
CCUGCACGAACACAUCGCAAACCUGGCAGGAAGCCCGGCAAUCAAGAAG
GGAAUCCUGCAGACAGUCAAGGUCGUCGACGAACUGGUCAAGGUCAUGG
GAAGACACAAGCCGGAAAACAUCGUCAUCGAAAUGGCAAGAGAAAACCA
GACAACACAGAAGGGACAGAAGAACAGCAGAGAAAGAAUGAAGAGAAUC
GAAGAAGGAAUCAAGGAACUGGGAAGCCAGAUCCUGAAGGAACACCCGG
UCGAAAACACACAGCUGCAGAACGAAAAGCUGUACCUGUACUACCUGCA
GAACGGAAGAGACAUGUACGUCGACCAGGAACUGGACAUCAACAGACUG
AGCGACUACGACGUCGACCACAUCGUCCCGCAGAGCUUCCUGAAGGACG
ACAGCAUCGACAACAAGGUCCUGACAAGAAGCGACAAGAACAGAGGAAA
GAGCGACAACGUCCCGAGCGAAGAAGUCGUCAAGAAGAUGAAGAACUAC
UGGAGACAGCUGCUGAACGCAAAGCUGAUCACACAGAGAAAGUUCGACA
ACCUGACAAAGGCAGAGAGAGGAGGACUGAGCGAACUGGACAAGGCAGG
AUUCAUCAAGAGACAGCUGGUCGAAACAAGACAGAUCACAAAGCACGUC
GCACAGAUCCUGGACAGCAGAAUGAACACAAAGUACGACGAAAACGACA
AGCUGAUCAGAGAAGUCAAGGUCAUCACACUGAAGAGCAAGCUGGUCAG
CGACUUCAGAAAGGACUUCCAGUUCUACAAGGUCAGAGAAAUCAACAAC
UACCACCACGCACACGACGCAUACCUGAACGCAGUCGUCGGAACAGCAC
UGAUCAAGAAGUACCCGAAGCUGGAAAGCGAAUUCGUCUACGGAGACUA
CAAGGUCUACGACGUCAGAAAGAUGAUCGCAAAGAGCGAACAGGAAAUC
GGAAAGGCAACAGCAAAGUACUUCUUCUACAGCAACAUCAUGAACUUCU
UCAAGACAGAAAUCACACUGGCAAACGGAGAAAUCAGAAAGAGACCGCU
GAUCGAAACAAACGGAGAAACAGGAGAAAUCGUCUGGGACAAGGGAAGA
GACUUCGCAACAGUCAGAAAGGUCCUGAGCAUGCCGCAGGUCAACAUCG
UCAAGAAGACAGAAGUCCAGACAGGAGGAUUCAGCAAGGAAAGCAUCCU
GCCGAAGAGAAACAGCGACAAGCUGAUCGCAAGAAAGAAGGACUGGGAC
CCGAAGAAGUACGGAGGAUUCGACAGCCCGACAGUCGCAUACAGCGUCC
UGGUCGUCGCAAAGGUCGAAAAGGGAAAGAGCAAGAAGCUGAAGAGCGU
CAAGGAACUGCUGGGAAUCACAAUCAUGGAAAGAAGCAGCUUCGAAAAG
AACCCGAUCGACUUCCUGGAAGCAAAGGGAUACAAGGAAGUCAAGAAGG
ACCUGAUCAUCAAGCUGCCGAAGUACAGCCUGUUCGAACUGGAAAACGG
AAGAAAGAGAAUGCUGGCAAGCGCAGGAGAACUGCAGAAGGGAAACGAA
CUGGCACUGCCGAGCAAGUACGUCAACUUCCUGUACCUGGCAAGCCACU
ACGAAAAGCUGAAGGGAAGCCCGGAAGACAACGAACAGAAGCAGCUGUU
CGUCGAACAGCACAAGCACUACCUGGACGAAAUCAUCGAACAGAUCAGC
GAAUUCAGCAAGAGAGUCAUCCUGGCAGACGCAAACCUGGACAAGGUCC
UGAGCGCAUACAACAAGCACAGAGACAAGCCGAUCAGAGAACAGGCAGA
AAACAUCAUCCACCUGUUCACACUGACAAACCUGGGAGCACCGGCAGCA
UUCAAGUACUUCGACACAACAAUCGACAGAAAGAGAUACACAAGCACAA
AGGAAGUCCUGGACGCAACACUGAUCCACCAGAGCAUCACAGGACUGUA
CGAAACAAGAAUCGACCUGAGCCAGCUGGGAGGAGACGGAGGAGGAAGC
CCGAAGAAGAAGAGAAAGGUCUAG

An exemplary Cas9 mRNA coding sequence, suitable for inclusion in a fusion protein, is provided as SEQ ID NO: 274.

(SEQ ID NO: 274)
GACAAGAAGUACAGCAUCGGACUGGACAUCGGAACAAACAGCGUCGGAU
GGGCAGUCAUCACAGACGAAUACAAGGUCCCGAGCAAGAAGUUCAAGGU
CCUGGGAAACACAGACAGACACAGCAUCAAGAAGAACCUGAUCGGAGCA
CUGCUGUUCGACAGCGGAGAAACAGCAGAAGCAACAAGACUGAAGAGAA
CAGCAAGAAGAAGAUACACAAGAAGAAAGAACAGAAUCUGCUACCUGCA
GGAAAUCUUCAGCAACGAAAUGGCAAAGGUCGACGACAGCUUCUUCCAC
AGACUGGAAGAAAGCUUCCUGGUCGAAGAAGACAAGAAGCACGAAAGAC
ACCCGAUCUUCGGAAACAUCGUCGACGAAGUCGCAUACCACGAAAAGUA
CCCGACAAUCUACCACCUGAGAAAGAAGCUGGUCGACAGCACAGACAAG
GCAGACCUGAGACUGAUCUACCUGGCACUGGCACACAUGAUCAAGUUCA
GAGGACACUUCCUGAUCGAAGGAGACCUGAACCCGGACAACAGCGACGU
CGACAAGCUGUUCAUCCAGCUGGUCCAGACAUACAACCAGCUGUUCGAA
GAAAACCCGAUCAACGCAAGCGGAGUCGACGCAAAGGCAAUCCUGAGCG
CAAGACUGAGCAAGAGCAGAAGACUGGAAAACCUGAUCGCACAGCUGCC
GGGAGAAAAGAAGAACGGACUGUUCGGAAACCUGAUCGCACUGAGCCUG
GGACUGACACCGAACUUCAAGAGCAACUUCGACCUGGCAGAAGACGCAA
AGCUGCAGCUGAGCAAGGACACAUACGACGACGACCUGGACAACCUGCU
GGCACAGAUCGGAGACCAGUACGCAGACCUGUUCCUGGCAGCAAAGAAC
CUGAGCGACGCAAUCCUGCUGAGCGACAUCCUGAGAGUCAACACAGAAA
UCACAAAGGCACCGCUGAGCGCAAGCAUGAUCAAGAGAUACGACGAACA
CCACCAGGACCUGACACUGCUGAAGGCACUGGUCAGACAGCAGCUGCCG
GAAAAGUACAAGGAAAUCUUCUUCGACCAGAGCAAGAACGGAUACGCAG
GAUACAUCGACGGAGGAGCAAGCCAGGAAGAAUUCUACAAGUUCAUCAA
GCCGAUCCUGGAAAAGAUGGACGGAACAGAAGAACUGCUGGUCAAGCUG
AACAGAGAAGACCUGCUGAGAAAGCAGAGAACAUUCGACAACGGAAGCA
UCCCGCACCAGAUCCACCUGGGAGAACUGCACGCAAUCCUGAGAAGACA
GGAAGACUUCUACCCGUUCCUGAAGGACAACAGAGAAAAGAUCGAAAAG
AUCCUGACAUUCAGAAUCCCGUACUACGUCGGACCGCUGGCAAGAGGAA
ACAGCAGAUUCGCAUGGAUGACAAGAAAGAGCGAAGAAACAAUCACACC
GUGGAACUUCGAAGAAGUCGUCGACAAGGGAGCAAGCGCACAGAGCUUC
AUCGAAAGAAUGACAAACUUCGACAAGAACCUGCCGAACGAAAAGGUCC
UGCCGAAGCACAGCCUGCUGUACGAAUACUUCACAGUCUACAACGAACU
GACAAAGGUCAAGUACGUCACAGAAGGAAUGAGAAAGCCGGCAUUCCUG
AGCGGAGAACAGAAGAAGGCAAUCGUCGACCUGCUGUUCAAGACAAACA
GAAAGGUCACAGUCAAGCAGCUGAAGGAAGACUACUUCAAGAAGAUCGA
AUGCUUCGACAGCGUCGAAAUCAGCGGAGUCGAAGACAGAUUCAACGCA
AGCCUGGGAACAUACCACGACCUGCUGAAGAUCAUCAAGGACAAGGACU
UCCUGGACAACGAAGAAAACGAAGACAUCCUGGAAGACAUCGUCCUGAC
ACUGACACUGUUCGAAGACAGAGAAAUGAUCGAAGAAAGACUGAAGACA
UACGCACACCUGUUCGACGACAAGGUCAUGAAGCAGCUGAAGAGAAGAA
GAUACACAGGAUGGGGAAGACUGAGCAGAAAGCUGAUCAACGGAAUCAG
AGACAAGCAGAGCGGAAAGACAAUCCUGGACUUCCUGAAGAGCGACGGA
UUCGCAAACAGAAACUUCAUGCAGCUGAUCCACGACGACAGCCUGACAU
UCAAGGAAGACAUCCAGAAGGCACAGGUCAGCGGACAGGGAGACAGCCU
GCACGAACACAUCGCAAACCUGGCAGGAAGCCCGGCAAUCAAGAAGGGA
AUCCUGCAGACAGUCAAGGUCGUCGACGAACUGGUCAAGGUCAUGGGAA
GACACAAGCCGGAAAACAUCGUCAUCGAAAUGGCAAGAGAAAACCAGAC
AACACAGAAGGGACAGAAGAACAGCAGAGAAAGAAUGAAGAGAAUCGAA
GAAGGAAUCAAGGAACUGGGAAGCCAGAUCCUGAAGGAACACCCGGUCG
AAAACACACAGCUGCAGAACGAAAAGCUGUACCUGUACUACCUGCAGAA
CGGAAGAGACAUGUACGUCGACCAGGAACUGGACAUCAACAGACUGAGC
GACUACGACGUCGACCACAUCGUCCCGCAGAGCUUCCUGAAGGACGACA
GCAUCGACAACAAGGUCCUGACAAGAAGCGACAAGAACAGAGGAAAGAG
CGACAACGUCCCGAGCGAAGAAGUCGUCAAGAAGAUGAAGAACUACUGG
AGACAGCUGCUGAACGCAAAGCUGAUCACACAGAGAAAGUUCGACAACC
UGACAAAGGCAGAGAGAGGAGGACUGAGCGAACUGGACAAGGCAGGAUU
CAUCAAGAGACAGCUGGUCGAAACAAGACAGAUCACAAAGCACGUCGCA
CAGAUCCUGGACAGCAGAAUGAACACAAAGUACGACGAAAACGACAAGC
UGAUCAGAGAAGUCAAGGUCAUCACACUGAAGAGCAAGCUGGUCAGCGA
CUUCAGAAAGGACUUCCAGUUCUACAAGGUCAGAGAAAUCAACAACUAC
CACCACGCACACGACGCAUACCUGAACGCAGUCGUCGGAACAGCACUGA
UCAAGAAGUACCCGAAGCUGGAAAGCGAAUUCGUCUACGGAGACUACAA
GGUCUACGACGUCAGAAAGAUGAUCGCAAAGAGCGAACAGGAAAUCGGA
AAGGCAACAGCAAAGUACUUCUUCUACAGCAACAUCAUGAACUUCUUCA
AGACAGAAAUCACACUGGCAAACGGAGAAAUCAGAAAGAGACCGCUGAU
CGAAACAAACGGAGAAACAGGAGAAAUCGUCUGGGACAAGGGAAGAGAC
UUCGCAACAGUCAGAAAGGUCCUGAGCAUGCCGCAGGUCAACAUCGUCA
AGAAGACAGAAGUCCAGACAGGAGGAUUCAGCAAGGAAAGCAUCCUGCC
GAAGAGAAACAGCGACAAGCUGAUCGCAAGAAAGAAGGACUGGGACCCG
AAGAAGUACGGAGGAUUCGACAGCCCGACAGUCGCAUACAGCGUCCUGG
UCGUCGCAAAGGUCGAAAAGGGAAAGAGCAAGAAGCUGAAGAGCGUCAA
GGAACUGCUGGGAAUCACAAUCAUGGAAAGAAGCAGCUUCGAAAAGAAC
CCGAUCGACUUCCUGGAAGCAAAGGGAUACAAGGAAGUCAAGAAGGACC
UGAUCAUCAAGCUGCCGAAGUACAGCCUGUUCGAACUGGAAAACGGAAG
AAAGAGAAUGCUGGCAAGCGCAGGAGAACUGCAGAAGGGAAACGAACUG
GCACUGCCGAGCAAGUACGUCAACUUCCUGUACCUGGCAAGCCACUACG
AAAAGCUGAAGGGAAGCCCGGAAGACAACGAACAGAAGCAGCUGUUCGU
CGAACAGCACAAGCACUACCUGGACGAAAUCAUCGAACAGAUCAGCGAA
UUCAGCAAGAGAGUCAUCCUGGCAGACGCAAACCUGGACAAGGUCCUGA
GCGCAUACAACAAGCACAGAGACAAGCCGAUCAGAGAACAGGCAGAAAA
CAUCAUCCACCUGUUCACACUGACAAACCUGGGAGCACCGGCAGCAUUC
AAGUACUUCGACACAACAAUCGACAGAAAGAGAUACACAAGCACAAAGG
AAGUCCUGGACGCAACACUGAUCCACCAGAGCAUCACAGGACUGUACGA
AACAAGAAUCGACCUGAGCCAGCUGGGAGGAGACGGAGGAGGAAGCCCG
AAGAAGAAGAGAAAGGUC

In some embodiments, chimeric Cas nucleases are used, where one domain or region of the protein is replaced by a portion of a different protein. In some embodiments, a Cas nuclease domain may be replaced with a domain from a different nuclease such as Fok 1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the Cas nuclease is fused to an exonuclease, for example, to reduce chromosomal translocations by suppressing repeated cleavage by promoting end processing and imperfect rejoinings. In some embodiments, the Cas nuclease is fused to TREX1, TREX2, or MRE11. In some embodiments, the exonuclease is truncated. In some embodiments, the Cas nuclease is fused to an exonuclease or a truncated exonuclease at the C terminus with a GGGGS (SEQ ID NO:374) linker. Cas exo-endonuclease fusions are described, for example, in Yin J, et al. Cas9 exo-endonuclease eliminates chromosomal translocations during genome editing. Nat Commun. 2022 Mar. 8; 13 (1): 1204. doi: 10.1038/s41467-022-28900-w. PMID: 35260581; PMCID: PMC8904484, which is hereby incorporated by reference in its entirety.

E. Determination of Efficacy of gRNAs

In some embodiments, the efficacy of a gRNA is determined when delivered together with other components, e.g., a nucleic acid encoding an RNA-guided DNA binding agent such as any of those described herein. In some embodiments, the efficacy of a combination of a gRNA and a nucleic acid encoding an RNA-guided DNA binding agent is determined.

As described herein, use of an RNA-guided DNA nuclease and a guide RNA disclosed herein can lead to double-stranded breaks in the DNA, which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular gRNAs or combinations is determined based on in vitro models. In some embodiments, the in vitro model is HEK293 cells. In some embodiments, the in vitro model is HUH7 human hepatocarcinoma cells. In some embodiments, the in vitro model is HepG2 cells. In some embodiments, the in vitro model is primary human hepatocytes. In some embodiments, the in vitro model is primary rodent hepatocytes. In some embodiments, the in vitro model is primary cynomolgus hepatocytes. With respect to using primary human hepatocytes, commercially available primary human hepatocytes can be used to provide greater consistency between experiments. In some embodiments, the number of off-target sites at which a deletion or insertion occurs in an in vitro model (e.g., in primary human hepatocytes) is determined, e.g., by analyzing genomic DNA from primary human hepatocytes transfected in vitro with Cas9 mRNA and the guide RNA In some embodiments, such a determination comprises analyzing genomic DNA from primary human hepatocytes transfected in vitro with Cas9 mRNA and the guide RNA. Exemplary procedures for such determinations are provided in the working examples below.

In some embodiments, the efficacy of particular gRNAs or combinations is determined across multiple in vitro cell models for a gRNA selection process. In some embodiments, a cell line comparison of data with selected gRNAs is performed. In some embodiments, cross screening in multiple cell models is performed.

In some embodiments, the efficacy of particular gRNAs or combinations is determined based on in vivo models. In some embodiments, the in vivo model is a rodent model. In some embodiments, the rodent model is a mouse, which expresses a human TTR gene, which may be a mutant human TTR gene. In some embodiments, the in vivo model is a non-human primate, for example, a cynomolgus monkey.

In some embodiments, the efficacy of a guide RNA or combination is measured by percent editing of TTR. In some embodiments, the percent editing of TTR is compared to the percent editing necessary to achieve knockdown of TTR protein, e.g., in the cell culture media in the case of an in vitro model or in serum or tissue in the case of an in vivo model. In some embodiments, the percent editing is between 30 and 99% of the population of cells. In some embodiments, the percent editing is between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the population of cells. In some embodiments, the percent editing is between 30%-95%, 40%-90%, or 50%-85%, 30%-60%, 40%-80%, 50%-75%, 60%-90%.

In some embodiments, the efficacy of a guide RNA or combination is measured by the number and/or frequency of indels at off-target sequences within the genome of the target cell type. In some embodiments, efficacious guide RNAs and combinations are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a hepatocyte), or which produce a frequency of off-target indel formation of <5% in a cell population and/or relative to the frequency of indel creation at the target site. In some embodiments, the disclosure provides guide RNAs and combinations which do not exhibit any off target indel formation in the target cell type (e.g., hepatocyte).

In some embodiments, guide RNAs and combinations are provided which produce indels at less than 5 off-target sites, e.g., as evaluated by one or more methods described herein. In some embodiments, guide RNAs and combinations are provided which produce indels at less than or equal to 4, 3, 2, or 1 off-target site(s), e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method), as described in WO2018/067447 or Schmidt et al., Nature Methods 4:1051-1057 (2007).

In some embodiments, detecting gene editing events, such as the formation of insertion/deletion (“indel”) mutations and homology directed repair (HDR) events in target DNA, further comprises sequencing the linear amplified products or the further amplified products. Sequencing may comprise any method known to those of skill in the art, including, next generation sequencing, and cloning the linear amplification products or further amplified products into a plasmid and sequencing the plasmid or a portion of the plasmid. Exemplary next generation sequencing methods are discussed, e.g., in Shendure et al., Nature 26:1135-1145 (2008). In other aspects, detecting gene editing events, such as the formation of insertion/deletion (“indel”) mutations and homology directed repair (HDR) events in target DNA, further comprises performing digital PCR (dPCR) or droplet digital PCR (ddPCR) on the linear amplified products or the further amplified products, or contacting the linear amplified products or the further amplified products with a nucleic acid probe designed to identify DNA having Homology-directed repair (HDR) template sequence and detecting the probes that have bound to the linear amplified product(s) or further amplified product(s). In some embodiments, the method further comprises determining the location of the HDR template in the target DNA.

In certain embodiments, the method further comprises determining the sequence of an insertion site in the target DNA, wherein the insertion site is the location where the HDR template incorporates into the target DNA, and wherein the insertion site may include some target DNA sequence and some HDR template sequence.

In some embodiments, the efficacy of a guide RNA or combination is measured by secretion of TTR. In some embodiments, secretion of TTR is measured using an enzyme-linked immunosorbent assay (ELISA) assay with cell culture media or serum. In some embodiments, secretion of TTR is measured in the same in vitro or in vivo systems or models used to measure editing. In some embodiments, secretion of TTR is measured in primary human hepatocytes. In some embodiments, secretion of TTR is measured in HUH7 cells. In some embodiments, secretion of TTR is measured in HepG2 cells. In some embodiments, secretion of TTR is measured in HEK cells.

ELISA assays are generally known to the skilled artisan and can be designed to determine serum TTR levels. In one exemplary embodiment, blood is collected and the serum is isolated. The total TTR serum levels may be determined using a Mouse Prealbumin (Transthyretin) ELISA Kit (Aviva Systems Biology) or a similar kit for measuring human TTR. If no kit is available, an ELISA can be developed using plates that are pre-coated with capture antibody specific for the TTR that is being measured. The plate is next incubated at room temperature for a period of time before washing. Enzyme-anti-TTR antibody conjugate is added and incubated. Unbound antibody conjugate is removed and the plate washed before the addition of the chromogenic substrate solution that reacts with the enzyme. The plate is read on an appropriate plate reader at an absorbance specific for the enzyme and substrate used.

In some embodiments, the amount of TTR in cells (including cells from tissue) measures efficacy of a gRNA or combination. In some embodiments, the amount of TTR in cells is measured using western blot. In some embodiments, the cell used is HUH7 cells. In some embodiments, the cell used is a primary human hepatocyte. In some embodiments, the cell used is a primary cell obtained from an animal. In some embodiments, the amount of TTR is compared to the amount of glyceraldehyde 3-phosphate dehydrogenase GAPDH (a housekeeping gene) to control for changes in cell number.

In some embodiments, the amount of TTR is reduced by between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the TTR in cells detected in the subject before administration of the composition. In some embodiments, the amount of TTR is reduced by between 30%-95%, 40%-90%, or 50%-85%, 30%-60%, 40%-80%, 50%-75%, or 60%-90% of the TTR in cells detected in the subject before administration of the composition.

In some embodiments, the degree or amount of amyloidosis is measured by methods known in the art. For example, amyloidosis in a subject can be measured using blood tests, urine tests and/or biopsies. Bone marrow tests or other small biopsy samples of tissue or organs can positively confirm the diagnosis of amyloidosis. In some embodiments, cardiac amyloidosis can be measured by cardiac biopsy, technetium pyrophosphate scans, or radionuclide imaging as described in Hotta M et al., RadioGraphics 2020; 40:2029-2041.

In some embodiments, amyloidosis is reduced by between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the amyloidosis in tissues detected in the subject before administration of the composition. In some embodiments, the amyloidosis is reduced by between 30%-95%, 40%-90%, or 50%-85%, 30%-60%, 40%-80%, 50%-75%, or 60%-90% of the amyloidosis in tissues detected in the subject before administration of the composition.

### F. Methods of Treatment

In some embodiments, the disclosure provides a method of treating ATTR is provided which includes administering a composition including a guide RNA having any one or more of the guide sequences of SEQ ID NOs: 1-81 or SEQ ID NOs: 275-307, or any one or more of the sgRNAs of SEQ ID NOs: 171-251 or SEQ ID NOs: 341-373, or any one or more of the crRNAs of SEQ ID NOs: 86-166 or SEQ ID NOs: 308-340. In some embodiments, the gRNAs have any one or more of the guide sequences of SEQ ID NOs: 1-81 or SEQ ID NOs: 275-307, or any one or more of the sgRNAs of SEQ ID NOs: 171-251 or SEQ ID NOs: 341-373 are administered to treat ATTR. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP having a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, the disclosure provides a method of inducing a double-stranded break (DSB) within the TTR gene including administering a composition having a guide RNA as described herein, e.g. having any one or more guide sequences of SEQ ID NOs: 1-81 or SEQ ID NOs: 275-307, or any one or more of the sgRNAs of SEQ ID NOs: 171-251 or SEQ ID NOs: 341-373. In some embodiments, gRNAs such as any one or more of the guide sequences of SEQ ID NOs: 1-81 or SEQ ID NOs: 275-307 are administered to recognize and bind to the TTR gene. The guide RNA is administered together with a nucleic acid (e.g., mRNA) or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, a method of inducing a double-stranded break (DSB) within the TTR gene is provided comprising administering a composition comprising a guide RNA, such as a chemically modified guide RNA, comprising any one or more guide sequences of SEQ ID NOs: 1-81 or SEQ ID NOs: 275-307, or any one or more of the sgRNAs of SEQ ID NOs: 171-251 or SEQ ID NOs: 341-373. In some embodiments, any one or more of the sgRNAs of SEQ ID NOs: 171-251 or SEQ ID NOs: 341-373 or gRNAs comprising any one or more of the guide sequences of SEQ ID NOs: 1-81 or SEQ ID NOs: 275-307 are administered to induce a DSB in the TTR gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, a method of modifying the TTR gene is provided comprising administering a composition comprising a guide RNA as described herein, e.g. having any one or more of the guide sequences of SEQ ID NOs: 1-81 or SEQ ID NOs: 275-307, or any one or more of the sgRNAs of SEQ ID NOs: 171-251 or SEQ ID NOs: 341-373. In some embodiments, gRNAs comprising any one or more of the guide sequences of SEQ ID NOs: 1-81 or SEQ ID NOs: 275-307, or any one or more of the sgRNAs of SEQ ID NOs: 171-251 or SEQ ID NOs: 341-373, are administered to modify the TTR gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, a method of modifying the TTR gene is provided comprising administering a composition comprising a guide RNA comprising any one or more of the guide sequences of SEQ ID NOs: 1-81 or SEQ ID NOs: 275-307, or any one or more of the sgRNAs of SEQ ID NOs: 171-251 or SEQ ID NOs: 341-373. In some embodiments, gRNAs comprising any one or more of the guide sequences of SEQ ID NOs: 1-81 or SEQ ID NOs: 275-307, or any one or more of the sgRNAs of SEQ ID NOs: 171-251 or SEQ ID NOs: 341-373, are administered to modify the TTR gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, a method of treating ATTR is provided comprising administering a composition comprising a guide RNA as described herein, e.g. having any one or more of the guide sequences of SEQ ID NOs: 1-81 or SEQ ID NOs: 275-307, or any one or more of the sgRNAs of SEQ ID NOs: 171-251 or SEQ ID NOs: 341-373. In some embodiments, gRNAs comprising any one or more of the guide sequences of SEQ ID NOs: 1-81 or SEQ ID NOs: 275-307, or any one or more of the sgRNAs of SEQ ID NOs: 171-251 or SEQ ID NOs: 341-373 are administered to treat ATTR. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, the disclosure features a method of reducing TTR serum concentration including administering a guide RNA as described herein, e.g. having any one or more of the guide sequences of SEQ ID NOs: 1-81 or SEQ ID NOs: 275-307, or any one or more of the sgRNAs of SEQ ID NOs: 171-251 or SEQ ID NOs: 341-373. In some embodiments, gRNAs comprising any one or more of the guide sequences of SEQ ID NOs: 1-81 or SEQ ID NOs: 275-307 or any one or more of the sgRNAs of SEQ ID NOs: 171-251 or SEQ ID NOs: 341-373 are administered to reduce or prevent the accumulation of TTR in amyloids or amyloid fibrils (amyloid deposition). The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, the disclosure features a method of reducing TTR serum concentration is provided including administering a guide RNA as described herein, e.g., comprising any one or more of the guide sequences of SEQ ID NOs: 1-81 or SEQ ID NOs: 275-307, or any one or more of the sgRNAs of SEQ ID NOs: 171-251 or SEQ ID NOs: 341-373. In some embodiments, gRNAs comprising any one or more of the guide sequences of SEQ ID NOs: 1-81 or SEQ ID NOs: 275-307, or any one or more of the sgRNAs of SEQ ID NOs: 171-251 or SEQ ID NOs: 341-373 are administered to reduce or prevent the accumulation of TTR in amyloids or amyloid fibrils (amyloid deposition). The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, the disclosure features a method of reducing or preventing the accumulation of TTR in amyloids or amyloid fibrils of a subject including comprising administering a composition comprising a guide RNA as described herein, e.g. having any one or more of the guide sequences of SEQ ID NOs: 1-81 or SEQ ID NOs: 275-307, or any one or more of the sgRNAs of SEQ ID NOs: 171-251 or SEQ ID NOs: 341-373. In some embodiments, a method of reducing or preventing the accumulation of TTR in amyloids or amyloid fibrils of a subject is provided comprising administering a composition comprising any one or more of the sgRNAs of SEQ ID NOs: 171-251 or SEQ ID NOs: 341-373. In some embodiments, gRNAs comprising any one or more of the guide sequences of SEQ ID NOs: 1-81 or SEQ ID NOs: 275-307 or any one or more of the sgRNAs of SEQ ID NOs: 171-251 or SEQ ID NOs: 341-373 are administered to reduce or prevent the accumulation of TTR in amyloids or amyloid fibrils (amyloid deposition). The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, the disclosure features a method of reducing or preventing the accumulation of TTR in amyloids or amyloid fibrils (amyloid deposition) of a subject including administering a composition comprising a guide RNA as described herein, e.g. having any one or more of the guide sequences of SEQ ID NOs: 1-81 or SEQ ID NOs: 275-307, or any one or more of the sgRNAs of SEQ ID NOs: 171-251 or SEQ ID NOs: 341-373. In some embodiments, a method of reducing or preventing the accumulation of

TTR in amyloids or amyloid fibrils (amyloid deposition) of a subject is provided comprising administering a composition comprising any one or more of the sgRNAs of SEQ ID NOs: 171-251 or SEQ ID NOs: 341-373. In some embodiments, gRNAs that include any one or more of the guide sequences of SEQ ID NOs: 1-81 or SEQ ID NOs: 275-307 or any one or more of the sgRNAs of SEQ ID NOs: 171-251 or SEQ ID NOs: 341-373 are administered to reduce or prevent the accumulation of TTR in amyloids or amyloid fibrils (amyloid deposition). The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP that includes a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

In some embodiments, the gRNA includes a guide sequence of Table 3 together with an RNA-guided DNA nuclease such as a Cas nuclease translated from the nucleic acid induce DSBs, and non-homologous ending joining (NHEJ) during repair leads to a mutation in the TTR gene. In some embodiments, NHEJ leads to a deletion or insertion of a nucleotide(s), which induces a frameshift or nonsense mutation in the TTR gene.

In some embodiments, administering the guide RNA and nucleic acid encoding an RNA-guided DNA binding agent (e.g., in a composition provided herein) reduces levels (e.g., serum levels) of TTR in the subject, and therefore prevents accumulation and aggregation of TTR in amyloids or amyloid fibrils (amyloid deposition).

In some embodiments, reducing or preventing the accumulation of TTR in amyloids or amyloid fibrils (amyloid deposition) of a subject comprises reducing or preventing TTR deposition in one or more tissues of the subject, such as liver, stomach, colon, or nervous tissue. In some embodiments, the nervous tissue comprises sciatic nerve or dorsal root ganglion. In some embodiments, TTR deposition is reduced in two, three, or four of the stomach, colon, dorsal root ganglion, and sciatic nerve. The level of deposition in a given tissue can be determined using a biopsy sample, e.g., using immunostaining. In some embodiments, reducing or preventing the accumulation of TTR in amyloids or amyloid fibrils of a subject and/or reducing or preventing TTR deposition is inferred based on reducing serum TTR levels for a period of time. As discussed in the examples, it has been found that reducing serum TTR levels in accordance with methods and uses provided herein can result in clearance of deposited TTR from tissues such as those discussed above and in the examples, e.g., as measured 8 weeks after administration of the composition.

In some embodiments, amyloid deposition reduced by between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the amyloid deposition seen in the subject before administration of the composition. In some embodiments, the amyloid deposition is reduced by between 30%-95%, 40%-90%, or 50%-85%, 30%-60%, 40%-80%, 50%-75%, or 60%-90% of the amyloid deposition seen in the subject before administration of the composition.

In some embodiments, the subject is mammalian. In some embodiments, the subject is human. In some embodiments, the subject is cow, pig, monkey, sheep, dog, cat, fish, or poultry. In some embodiments, the subject is a companion animal or a livestock animal.

In some embodiments, the use of one or more guide RNAs as described herein, e.g. including any one or more of the guide sequences in Table 3 (e.g., in a composition provided herein) and of a nucleic acid (e.g. mRNA) described herein encoding an RNA-guided DNA-binding agent is provided for the preparation of a medicament for treating a human subject having ATTR. The RNA-guided DNA-binding agent may be a Cas9, e.g. an S. pyogenes Cas9. In particular embodiments, the guide RNA is chemically modified.

In some embodiments, the composition that includes the guide RNA and nucleic acid is administered intravenously. In some embodiments, the composition that includes the guide RNA and nucleic acid is administered into the hepatic circulation.

In some embodiments, a single administration of a composition that includes a guide RNA and nucleic acid provided herein is sufficient to knock down expression of the mutant protein. In some embodiments, a single administration of a composition that includes a guide RNA and nucleic acid provided herein is sufficient to knock out expression of the mutant protein in a population of cells. In other embodiments, more than one administration of a composition that includes a guide RNA and nucleic acid provided herein may be beneficial to maximize editing via cumulative effects. For example, a composition provided herein can be administered 2, 3, 4, 5, or more times, such as 2 times. Administrations can be separated by a period of time ranging from, e.g., 1 day to 2 years, such as 1 to 7 days, 7 to 14 days, 14 days to 30 days, 30 days to 60 days, 60 days to 120 days, 120 days to 183 days, 183 days to 274 days, 274 days to 366 days, or 366 days to 2 years.

In some embodiments, a composition is administered in an effective amount in the range of 0.01 to 10 mg/kg (mpk), e.g., 0.01 to 0.1 mpk, 0.1 to 0.3 mpk, 0.3 to 0.5 mpk, 0.5 to 1 mpk, 1 to 2 mpk, 2 to 3 mpk, 3 to 5 mpk, 5 to 10 mpk, or 0.1, 0.2, 0.3, 0.5, 1, 2, 3, 5, or 10 mpk. In some embodiments, a composition is administered in the amount of 2-4 mg/kg, such as 2.5-3.5 mg/kg. In some embodiments, a composition is administered in the amount of about 3 mg/kg.

In some embodiments, the efficacy of treatment with the compositions described herein is assessed at 1 year, 2 years, 3 years, 4 years, 5 years, or 10 years after delivery. In some embodiments, efficacy of treatment with the compositions described herein is assessed by measuring serum levels of TTR (monomer and/or tetramer) before and after treatment. In some embodiments, efficacy of treatment with the compositions assessed via a reduction of serum levels of TTR is seen at 1 week, 2 weeks, 3 weeks, 4 weeks, 2 months, 3 months, 4 months, 5 months, 6 months, 7 months, 8 months, 9 months, 10 months, or at 11 months. In some embodiments, the levels of monomeric, dimeric, and/or tetrameric TTR are reduced by at least 10%, at least 20%, at least 30%, at least 40%, at least 50%, at least 60%, at least 70%, at least 80%, at least 90%, at least 95%, or at least 99%.

In some embodiments, treatment slows or halts disease progression.

In some embodiments, treatment slows or halts progression of FAP. In some embodiments, treatment results in improvement, stabilization, or slowing of change in symptoms of sensorimotor neuropathy or autonomic neuropathy.

In some embodiments, treatment results in improvement, stabilization, or slowing of change in symptoms of FAC. In some embodiments, treatment results in improvement, stabilization, or slowing of change symptoms of restrictive cardiomyopathy or congestive heart failure.

In some embodiments, efficacy of treatment is measured by increased survival time of the subject.

In some embodiments, efficacy of treatment is measured by improvement or slowing of progression in symptoms of sensorimotor or autonomic neuropathy. In some embodiments, efficacy of treatment is measured by an increase or a slowing of decrease in ability to move an area of the body or to feel in any area of the body. In some embodiments, efficacy of treatment is measured by improvement or a slowing of decrease in the ability to swallow; breathe; use arms, hands, legs, or feet; or walk. In some embodiments, efficacy of treatment is measured by improvement or a slowing of progression of neuralgia. In some embodiments, the neuralgia is characterized by pain, burning, tingling, or abnormal feeling.

In some embodiments, efficacy of treatment is measured by improvement or a slowing of increase in postural hypotension, dizziness, gastrointestinal dysmotility, bladder dysfunction, or sexual dysfunction. In some embodiments, efficacy of treatment is measured by improvement or a slowing of progression of weakness. In some embodiments, efficacy of treatment is measured using electromyogram, nerve conduction tests, or patient-reported outcomes.

In some embodiments, efficacy of treatment is measured by improvement or slowing of progression of symptoms of congestive heart failure or CHF. In some embodiments, efficacy of treatment is measured by a decrease or a slowing of increase in shortness of breath, trouble breathing, fatigue, or swelling in the ankles, feet, legs, abdomen, or veins in the neck. In some embodiments, efficacy of treatment is measured by improvement or a slowing of progression of fluid buildup in the body, which may be assessed by measures such as weight gain, frequent urination, or nighttime cough. In some embodiments, efficacy of treatment is measured using cardiac biomarker tests (such as 13-type natriuretic peptide [BNP] or N-terminal pro b-type natriuretic peptide [NT-proBNP]), lung function tests, chest x-rays, or electrocardiography.

### Additional Treatments

In some embodiments, combination therapies are described that include administering any one of the gRNAs as described herein, e.g., including any one or more of the guide sequences disclosed in Table 3 and a nucleic acid encoding an RNA-guided DNA-binding agent (e.g., in a composition provided herein) as described herein, such as a nucleic acid (e.g. mRNA) or vector described herein encoding an S. pyogenes Cas9, together with an additional therapy suitable for alleviating symptoms of ATTR.

In some embodiments, the additional therapy for ATTR is a treatment for sensorimotor or autonomic neuropathy. In some embodiments, the treatment for sensorimotor or autonomic neuropathy is a nonsteroidal anti-inflammatory drug, antidepressant, anticonvulsant medication, antiarrythmic medication, or narcotic agent. In some embodiments, the antidepressant is a tricyclic agent or a serotonin-norepinephrine reuptake inhibitor. In some embodiments, the antidepressant is amitriptyline, duloxetine, or venlafaxine. In some embodiments, the anticonvulsant agent is gabapentin, pregabalin, topiramate, or carbamazepine. In some embodiments, the additional therapy for sensorimotor neuropathy is transcutaneous electrical nerve stimulation.

In some embodiments, the additional therapy for ATTR is a treatment for restrictive cardiomyopathy or congestive heart failure (CHF). In some embodiments, the treatment for CHF is an ACE inhibitor, aldosterone antagonist, angiotensin receptor blocker, beta blocker, digoxin, diuretic, or isosorbide dinitrate/hydralazine hydrochloride. In some embodiments, the ACE inhibitor is enalapril, captopril, ramipril, perindopril, imidapril, or quinapril. In some embodiments, the aldosterone antagonist is eplerenone or spironolactone. In some embodiments, the angiotensin receptor blocker is azilsartan, cadesartan, eprosartan, irbesartan, losartan, olmesartan, telmisartan, or valsartan. In some embodiments, the beta blocker is acebutolol, atenolol, bisoprolol, metoprolol, nadolol, nebivolol, or propranolol. In some embodiments, the diuretic is chlorothiazide, chlorthalidone, hydrochlorothiazide, indapamide, metolazone, bumetanide, furosemide, torsemide, amiloride, or triameterene.

In some embodiments, the combination therapy comprises administering any one of the gRNAs that includes any one or more of the guide sequences disclosed in Table 3 and a nucleic acid encoding an RNA-guided DNA-binding agent (e.g., in a composition provided herein) together with a siRNA that targets TTR or mutant TTR. In some embodiments, the siRNA is any siRNA capable of further reducing or eliminating the expression of wild type or mutant TTR. In some embodiments, the siRNA is the drug Patisiran (ALN-TTR02) or ALN-TTRsc02. In some embodiments, the siRNA is administered after any one of the gRNAs that includes any one or more of the guide sequences disclosed in Table 3 (e.g., in a composition provided herein). In some embodiments, the siRNA is administered on a regular basis following treatment with any of the gRNA compositions provided herein.

In some embodiments, the combination therapy comprises administering any one of the gRNAs that includes any one or more of the guide sequences described herein, e.g., disclosed in Table 3 and a nucleic acid encoding an RNA-guided DNA-binding agent described herein (e.g., in a composition provided herein) together with antisense nucleotide that targets TTR or mutant TTR. In some embodiments, the antisense nucleotide is any antisense nucleotide capable of further reducing or eliminating the expression of wild type or mutant TTR. In some embodiments, the antisense nucleotide is the drug Inotersen (IONS-TTRRx). In some embodiments, the antisense nucleotide is administered after any one of the gRNAs that includes any one or more of the guide sequences disclosed in Table 3 and a nucleic acid encoding an RNA-guided DNA-binding agent (e.g., in a composition provided herein). In some embodiments, the antisense nucleotide is administered on a regular basis following treatment with any of the gRNA compositions provided herein.

In some embodiments, the combination therapy comprises administering any one of the gRNAs that includes any one or more of the guide sequences disclosed in Table 3 and a nucleic acid encoding an RNA-guided DNA-binding agent (e.g., in a composition provided herein) together with a small molecule stabilizer that promotes kinetic stabilization of the correctly folded tetrameric form of TTR.

In some embodiments, the small molecule stabilizer is the drug tafamidis (Vyndaqel®) or diflunisal. In some embodiments, the small molecule stabilizer is administered after any one of the gRNAs that includes any one or more of the guide sequences disclosed in Table 3 (e.g., in a composition provided herein). In some embodiments, the small molecule stabilizer is administered on a regular basis following treatment with any of the compositions provided herein.

In some embodiments, the combination therapy comprises administering any one of the gRNAs that includes any one or more of the guide sequences disclosed in Table 3 and a nucleic acid encoding an RNA-guided DNA-binding agent (e.g., in a composition provided herein) together with any of the compounds disclosed in Müller M L et al., European Journal of Heart Failure (2020) 22, 39-53 For instance, the compound is a TTR tetramer stabilizer such as Tafamidis (Vyndaqel), Diflunisal, or Epigallocatechin-3-gallate (EGCG), a TTR silencer such as Inotersen (Tegsedi) or Patisiran (Onpattro), a fibril disruptor such as Doxycycline, tauroursodeoxycholic acid (TUDCA) or a monoclonal antibody such as an anti-TTR antibody or an anti-Serum Amyloid P (SAP) antibody (Dezamizumab).

In any of the foregoing embodiments, the guide sequences disclosed in Table 3, and/or the guide RNA may be a chemically modified guide RNA.

In some embodiments, a method described herein comprises infusion prophylaxis. In some embodiments, an infusion prophylaxis is administered to a subject before the gene editing composition. In some embodiments, an infusion prophylaxis is administered to a subject 8-24 hours or 1-2 hours prior to the administration of the nucleic acid composition.

In some embodiments, an infusion prophylaxis comprises corticosteroid. In some embodiments, the infusion prophylaxis comprises one or more, or all, of corticosteroid, an antipyretic (e.g. oral acetaminophen (also called paracetamol), which may reduce pain and fever and/or inhibit COX enzymes and/or prostaglandins), H1 blocker, or H2 blocker. In some embodiments, the infusion prophylaxis comprises an intravenous corticosteroid (e.g., dexamethasone 8-12 mg, such as 10 mg or equivalent) and an antipyretic (e.g. oral acetaminophen or paracetamol 500 mg). In some embodiments, the H1 blocker (e.g., diphenhydramine 50 mg or equivalent) and/or H2 blocker (e.g., ranitidine 50 mg or equivalent) are administered orally. In some embodiments, the H1 blocker (e.g., diphenhydramine 50 mg or equivalent) and/or H2 blocker (e.g., ranitidine 50 mg or equivalent) are administered intravenously. In some embodiments, an infusion prophylaxis is administered intravenously 1-2 hour before infusion of the nucleic acid composition.

In some embodiments an intravenous H1 blocker and/or an intravenous H2 blocker is substituted with an oral equivalent. The infusion prophylaxis may function to reduce adverse reactions associated with administering the nucleic acid composition. In some embodiments, the infusion prophylaxis is administered as a required premedication prior to administering the nucleic acid composition. The dosage, frequency and mode of administration of the corticosteroid, infusion prophylaxis, and the guide-RNA containing composition described herein can be controlled independently.

The corticosteroid used in the disclosed methods may be administered according to regimens known in the art, e.g., US FDA-approved regimens. In some embodiments, e.g., administration to or for use in a human subject, the corticosteroid can be administered in an amount that ranges from about 0.75 mg to about 25 mg. In some embodiments, e.g., administration to or for use in a human subject, the corticosteroid can be administered in an amount that ranges from about 0.01-0.5 mg/kg, such as 0.1-0.40 mg/kg or 0.25-0.40 mg/kg.

In some embodiments, the corticosteroid is administered before the guide RNA-containing composition described herein. In some embodiments, the corticosteroid is administered after the guide RNA-containing composition described herein. In some embodiments, the corticosteroid is administered simultaneously with the guide RNA-containing composition described herein. In some embodiments, multiple doses of the corticosteroid are administered before or after the administration of the guide RNA-containing composition. In some embodiments, multiple doses of the guide RNA-containing composition are administered before or after the administration of the corticosteroid. In some embodiments, multiple doses of the corticosteroid and multiple doses of the guide RNA-containing composition are administered.

If appropriate, a dose of corticosteroid may be administered as at least two sub doses administered separately at appropriate intervals. In some embodiments, the corticosteroid is administered at least two times before the administration of the guide RNA-containing composition described herein. In some embodiments, a dose of corticosteroid is administered at least two times after the administration of the guide RNA-containing composition described herein. In some embodiments, the corticosteroid is administered (e.g., before, with, and/or after the administration of the guide RNA-containing composition described herein) at an interval of 1 hour, 2 hours, 3 hours, 4 hours, 6 hours, 12 hours, 18 hours; 1, 2, 3, 4, 5, 6, 7, 8, 9, 10, 11, 12, 13, 14, or 15 days; 3, 4, 5, 6, 7, 8, 9, 10, 11, 12, 13, 14, or 15 weeks; or an amount of time in a range bounded by any two of the preceding values. In some embodiments, the corticosteroid is administered before the administration of the guide RNA-containing composition described herein at an interval of 1 hour, 2 hours, 3 hours, 4 hours, 6 hours, 12 hours, 18 hours; 1, 2, 3, 4, 5, 6, 7, 8, 9, 10, 11, 12, 13, 14, 15 days; 3, 4, 5, 6, 7, 8, 9, 10, 11, 12, 13, 14, or 15 weeks; or an amount of time in a range bounded by any two of the preceding values. In some embodiments, the corticosteroid is administered after the administration of the guide RNA-containing composition described herein at an interval of 1 hour, 2 hours, 3 hours, 4 hours, 6 hours, 12 hours, 18 hours; 1, 2, 3, 4, 5, 6, 7, 8, 9, 10, 11, 12, 13, 14, 15 days; 3, 4, 5, 6, 7, 8, 9, 10, 11, 12, 13, 14, or 15 weeks; or an amount of time in a range bounded by any two of the preceding values.

In some embodiments, the corticosteroid is administered at least two times. In some embodiments, the corticosteroid is administered at least three times. In some embodiments, the corticosteroid is administered at least four times. In some embodiments, the corticosteroid is administered up to five, six, seven, eight, nine, or ten times. A first dose may be oral and a second or subsequent dose may be by parenteral administration, e.g. infusion. Alternatively, a first dose may be parenteral and a second or subsequent dose may be by oral administration.

In some embodiments, the corticosteroid is administered orally before intravenous administration of a guide RNA-containing composition described herein. In some embodiments, the corticosteroid is administered orally at or after intravenous administration of a guide RNA-containing composition described herein.

In some embodiments, corticosteroid is dexamethasone. In some embodiments, dexamethasone is administered intravenously 1-2 hour before infusion of the nucleic acid composition. In some embodiments, dexamethasone is administered intravenously in the amount of 8-12 mg, such as 10 mg, 1-2 hour before infusion of the nucleic acid composition. In some embodiments, dexamethasone is administered orally 8 to 24 hours before infusion of the nucleic acid composition. In some embodiments, dexamethasone is administered orally in the amount of 8-12 mg, such as 8 mg, 8 to 24 hours before infusion of the nucleic acid composition. In some embodiments, dexamethasone is administered orally in the amount of 8-12 mg, such as 8 mg, 8 to 24 hours before infusion of the nucleic acid composition and dexamethasone is administered intravenously in the amount of 8-12 mg, such as 10 mg, 1-2 hour before infusion of the nucleic acid composition.

### Delivery of Nucleic Acid Compositions

In some embodiments, the nucleic acid compositions described herein, that include a gRNA and a nucleic acid encoding an RNA-guided DNA-binding agent as RNA or encoded on one or more vectors, are formulated in or administered via a lipid nanoparticle (LNP); see e.g., WO2017173054A1 and WO2019067992A1, the contents of which are hereby incorporated by reference in their entireties. Any LNP known to those of skill in the art to be capable of delivering nucleotides to subjects may be utilized with the guide RNAs described herein and the nucleic acid encoding an RNA-guided DNA nuclease.

In some embodiments, the guide RNA and the nucleic acid encoding an RNA-guided DNA nuclease are administered in an LNP described herein, such as an LNP that includes a CCD lipid (e.g., an amine lipid), a helper lipid (e.g., cholesterol), a stealth lipid (e.g., a PEG lipid, such as PEG2k-DMG), and optionally a neutral lipid (e.g., DSPC).

Disclosed herein are various embodiments of LNP formulations for RNAs, including CRISPR/Cas cargoes. Such LNP formulations may include (i) a CCD lipid, such as an amine lipid, (ii) a neutral lipid, (iii) a helper lipid, and (iv) a stealth lipid, such as a PEG lipid. Some embodiments of the LNP formulations include an amine lipid, along with a helper lipid, a neutral lipid, and a stealth lipid such as a PEG lipid. In some embodiments, the LNP formulations include less than 1 percent neutral phospholipid. In some embodiments, the LNP formulations include less than 0.5 percent neutral phospholipid. A “lipid nanoparticle” could be a particle that comprises a plurality of (i.e. more than one) lipid molecules physically associated with each other by intermolecular forces. CCD Lipids, Amine Lipids, Neutral Lipids, and other lipids that can be used in the LNP formulations disclosed herein are described in WO2020198697, WO2015006747, WO2016118724, and WO2021026358, each of which is incorporated herein in its entirety.

In some embodiments, the nucleic acid compositions described herein are formulated in or administered via a lipid nanoparticle (LNP) which comprises ALC0315, DSPC, cholesterol, and DMG-PEG2000. In some embodiments, the N/P ratio of the LNP is about 1, 2, 3, 4, 5, 6, 7, 8, 9, or 10. In some embodiments, the N/P ratio is about 3 to about 7. The N/P ratio is the molar ratio between amines (N, which become cationic at low pH) found on the ionizable lipids, and the phosphates (P, anionic) found on the RNA backbone. The N/P ratio can affect the encapsulation efficiency and biological activity of LNP formulations comprising RNAs.

In some embodiments, the ALC0315 comprises from 40 mol % to 60 mol % of the total lipid present in the particle, DSPC comprises from 5 mol % to 15 mol % of the total lipid present in the particle, cholesterol comprises from 30 mol % to 50 mol % of the total lipid present in the particle, and DMG-PEG2000 comprises from 1 mol % to 5 mol % total lipid present in the particle. In an embodiments, the LNP comprises ALC0315, DSPC, cholesterol, and DMG-PEG2000 at a molar ratio of 50:9.5:37.5:3.

Further technologies that can be used for delivery of the compositions of this disclosure include those that utilize encapsulation by biodegradable polymers, liposomes, or nanoparticles. In some embodiments, the compositions of this disclosure are administered in any suitable delivery vehicle, including, but not limited to, polymers, engineered viral particles (e.g., adeno-associated virus), exosomes, liposomes, supercharged proteins, implantable devices, or red blood cells. Suitable delivery methods are described in U.S. Pat. Nos. 10,851,357, 10,709,797, and US20170349914, each of which is incorporated herein in its entirety.

### EXAMPLES

The practice of the methods and compositions of the disclosure employs, unless otherwise indicated, conventional techniques of molecular biology (including recombinant techniques), cell culture, immunology, cell biology, and biochemistry, which are well within the purview of the skilled artisan. Such techniques are explained in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The materials, reagents, and methods, further described below, are used in the following examples. The embodiments described in the following examples do not limit the scope of the claims.

### Example 1. Design of Guide RNA Sequences

Initial guide selection was performed in silico using CRISPOR (CRISPOR (tefor.net)), a web tool for genome editing experiments with the CRISPR-Cas9 system. CRISPOR finds guide RNAs in an input sequence and ranks them according to different scores that evaluate potential off-targets in the genome of interest and predict on-target activity. Initial guide selection was done using a human reference genome—Homo sapiens (human) genome assembly GRCh38.p13 (hg38) and user defined genomic regions of interest (e.g., TTR protein coding exons, promoter, regulatory region, etc.), for identifying PAMs (NGG) in the regions of interest. For each selected PAM, analyses were performed and statistics reported. gRNA molecules were further selected and rank ordered based on a number of criteria (e.g., GC content, predicted on-target activity, and potential off-target activity). ART-Design refers to the guide sequences that were prepared. Table 3 below shows these 118 guide sequences that were designed to be targeted to the TTR gene. The corresponding sgRNAs are shown in Table 4.

TABLE 3
Guide sequences

| | | | Chromosomal | SEQ ID |
|---|---|---|---|---|
| Name | Guide Sequence | Description | Location | NO |
| ART-001-g-006 | CCCAUUCCAUGAGCAUGCAG | TTR (Exon 3) | chr18: 31595234- | 1 |
| | | | 31595253 | |
| ART-001-g-012 | GCCAUCCUGCCAAGAAUGAG | TTR (Exon 1) | chr18: 31591888- | 2 |
| | | | 31591907 | |
| ART-001-g-015 | GAAUGAGUGGACUUCUGUGA | TTR (Exon 1) | chr18: 31591875- | 3 |
| | | | 31591894 | |
| ART-001-g-017 | UCCUAUAAGGUGUGAAAGUC | TTR (Intron) | chr18: 31595102- | 4 |
| | | | 31595121 | |
| ART-001-g-018 | GUGAGUAUACAGACCUUCGA | TTR (Intron) | chr18: 31595256- | 5 |
| | | | 31595275 | |
| ART-001-g-019 | GGUGAGUAUACAGACCUUCG | TTR (Intron) | chr18: 31595255- | 6 |
| | | | 31595274 | |
| ART-001-g-020 | AUGAGGGACUUCUCCUCCAG | TTR (Exon 4) | chr18: 31598672- | 7 |
| | | | 31598691 | |
| ART-001-g-021 | UUCUCCUCCAGUGGACCUGA | TTR (Exon 4) | chr18: 31598681- | 8 |
| | | | 31598700 | |
| ART-001-g-022 | CCAGUGGACCUGAAGGACGA | TTR (Exon 4) | chr18: 31598688- | 9 |
| | | | 31598707 | |
| ART-001-g-023 | UCCAGUGGACCUGAAGGACG | TTR (Exon 4) | chr18: 31598687- | 10 |
| | | | 31598706 | |
| ART-001-g-024 | UCGUCCUUCAGGUCCACUGG | TTR (Exon 4) | chr18: 31598688- | 11 |
| | | | 31598707 | |
| ART-001-g-025 | AAUCCCAUCCCUCGUCCUUC | TTR (Exon 4) | chr18: 31598699- | 12 |
| | | | 31598718 | |
| ART-001-g-026 | CCCUCGUCCUUCAGGUCCAC | TTR (Exon 4) | chr18: 31598691- | 13 |
| | | | 31598710 | |
| ART-001-g-027 | UGGACCUGAAGGACGAGGGA | TTR (Exon 4) | chr18: 31598692- | 14 |
| | | | 31598711 | |
| ART-001-g-028 | GGACCUGAAGGACGAGGGAU | TTR (Exon 4) | chr18: 31598693- | 15 |
| | | | 31598712 | |
| ART-001-g-029 | UAGUAAAAAUGGAAUACUCU | TTR (Exon 4) | chr18: 31598730- | 16 |
| | | | 31598749 | |
| ART-001-g-030 | AAACACUGCUUUAGUAAAAA | TTR (Exon 4) | chr18: 31598741- | 17 |
| | | | 31598760 | |
| ART-001-g-031 | GACUUCUAACAUAGCAUAUG | TTR (Exon 4) | chr18: 31598767- | 18 |
| | | | 31598786 | |
| ART-001-g-032 | AUAUGCUAUGUUAGAAGUCC | TTR (Exon 4) | chr18: 31598768- | 19 |
| | | | 31598787 | |
| ART-001-g-033 | AAUGUUUUAUUGUCUCUGCC | TTR (Exon 4) | chr18: 31598789- | 20 |
| | | | 31598808 | |
| ART-001-g-034 | CAAUAAAACAUUCCUGUGAA | TTR (Exon 4) | chr18: 31598797- | 21 |
| | | | 31598816 | |
| ART-001-g-035 | GAAUGAAAAGUGCCUUUCAC | TTR (Exon 4) | chr18: 31598812- | 22 |
| | | | 31598831 | |
| ART-001-g-039 | AGUCAAUAAUCAGAAUCAGC | TTR | chr18: 31591775- | 23 |
| | | (Promoter) | 31591794 | |
| ART-001-g-040 | CAGCAGGUUUGCAGUCAGAU | TTR | chr18: 31591791- | 24 |
| | | (Promoter) | 31591810 | |
| ART-001-g-041 | AGGUUUGCAGUCAGAUUGGC | TTR | chr18: 31591795- | 25 |
| | | (Promoter) | 31591814 | |
| ART-001-g-042 | GGUUUGCAGUCAGAUUGGCA | TTR | chr18: 31591796- | 26 |
| | | (Promoter) | 31591815 | |
| ART-001-g-043 | AGGGAUAAGCAGCCUAGCUC | TTR | chr18: 31591815- | 27 |
| | | (Promoter) | 31591834 | |
| ART-001-g-044 | AUACUCACUUCUCCUGAGCU | TTR | chr18: 31591830- | 28 |
| | | (Promoter) | 31591849 | |
| ART-001-g-045 | GAAGUGAGUAUAAAAGCCCC | TTR | chr18: 31591839- | 29 |
| | | (Promoter) | 31591858 | |
| ART-001-g-046 | UGAGUAUAAAAGCCCCAGGC | TTR | chr18: 31591843- | 30 |
| | | (Promoter) | 31591862 | |
| ART-001-g-047 | GAGUAUAAAAGCCCCAGGCU | TTR | chr18: 31591844- | 31 |
| | | (Promoter) | 31591863 | |
| ART-001-g-048 | UGAUGGCUGCUCCCAGCCUG | TTR | chr18: 31591858- | 32 |
| | | (Promoter) | 31591877 | |
| ART-001-g-049 | GUGAUGGCUGCUCCCAGCCU | TTR | chr18: 31591859- | 33 |
| | | (Promoter) | 31591878 | |
| ART-001-g-050 | UGUGAUGGCUGCUCCCAGCC | TTR | chr18: 31591860- | 34 |
| | | (Promoter) | 31591879 | |
| ART-001-g-051 | UUUAAAAAAUCAAGUUAAAG | TTR (3′RR) | chr18: 31598834- | 35 |
| | | | 31598853 | |
| ART-001-g-052 | UUUUUUGGAAGGGACAAUAA | TTR (3′RR) | chr18: 31598859- | 36 |
| | | | 31598878 | |
| ART-001-g-053 | UUUUUUUGGAAGGGACAAUA | TTR (3′RR) | chr18: 31598860- | 37 |
| | | | 31598879 | |
| ART-001-g-054 | UGAUUCUCUUUUUUUUGGAA | TTR (3′RR) | chr18: 31598869- | 38 |
| | | | 31598888 | |
| ART-001-g-055 | UUGAUUCUCUUUUUUUUGGA | TTR (3′RR) | chr18: 31598870- | 39 |
| | | | 31598889 | |
| ART-001-g-056 | AAUUUUGAUUCUCUUUUUUU | TTR (3′RR) | chr18: 31598874- | 40 |
| | | | 31598893 | |
| ART-001-g-057 | AAAAUUUUACAAAGAAUCAA | TTR (3′RR) | chr18: 31598888- | 41 |
| | | | 31598907 | |
| ART-001-g-058 | AAGGAAUUCUAGAAAGUAUC | TTR (3′RR) | chr18: 31598907- | 42 |
| | | | 31598926 | |
| ART-001-g-059 | AGGAAUUCUAGAAAGUAUCU | TTR (3′RR) | chr18: 31598908- | 43 |
| | | | 31598927 | |
| ART-001-g-060 | AAGUAUCUGGGCAGAACGCU | TTR (3′RR) | chr18: 31598920- | 44 |
| | | | 31598939 | |
| ART-001-g-061 | CUUGCAAGACAAUGGAAAUU | TTR (3′RR) | chr18: 31598952- | 45 |
| | | | 31598971 | |
| ART-001-g-062 | GUGCUUUGCUUGCAAGACAA | TTR (3′RR) | chr18: 31598960- | 46 |
| | | | 31598979 | |
| ART-001-g-063 | UACAGAAUGUGUCUUUUUAA | TTR (3′RR) | chr18: 31599003- | 47 |
| | | | 31599022 | |
| ART-001-g-001 | UGUGUCUGAGGCUGGCCCUA | TTR (Exon 1) | chr18: 31591950- | 48 |
| | | | 31591969 | |
| ART-001-g-002 | CUUGGAUUCACCGGUGCCCU | TTR (Exon 2) | chr18: 31592894- | 49 |
| | | | 31592913 | |
| ART-001-g-003 | ACUUGGAUUCACCGGUGCCC | TTR (Exon 2) | chr18: 31592895- | 50 |
| | | | 31592914 | |
| ART-001-g-004 | UCCAGACUUUCACACCUUAU | TTR (Intron) | chr18: 31595098- | 51 |
| | | | 31595117 | |
| ART-001-g-005 | GACUCACUGGUUUUCCUAUA | TTR (Exon 3) | chr18: 31595115- | 52 |
| | | | 31595134 | |
| ART-001-g-007 | AUCUGUCUGUCUUCUCUCAU | TTR (Intron) | chr18: 31598546- | 53 |
| | | | 31598565 | |
| ART-001-g-008 | GUCACCAAUCCCAAGGAAUG | TTR (Exon 4) | chr18: 31598655- | 54 |
| | | | 31598674 | |
| ART-001-g-009 | UCACCAAUCCCAAGGAAUGA | TTR (Exon 4) | chr18: 31598656- | 55 |
| | | | 31598675 | |
| ART-001-g-010 | AGGAGAAGUCCCUCAUUCCU | TTR (Exon 4) | chr18: 31598668- | 56 |
| | | | 31598687 | |
| ART-001-g-011 | GGAGAAGUCCCUCAUUCCUU | TTR (Exon 4) | chr18: 31598667- | 57 |
| | | | 31598686 | |
| ART-001-g-013 | GAAGUCCACUCAUUCUUGGC | TTR (Exon 1) | chr18: 31591880- | 58 |
| | | | 31591899 | |
| ART-001-g-014 | CACAGAAGUCCACUCAUUCU | TTR (Exon 1) | chr18: 31591876- | 59 |
| | | | 31591895 | |
| ART-001-g-int01 | GGGAGCUGCUGAUGACACCU | TTR (artificial) | chr18: 31592992- | 60 |
| | | | 31593007 | |
| ART-001-g-int02 | AGGAGCUGCUGAUGACACCU | TTR (artificial) | chr18: 31592992- | 61 |
| | | | 31593007 | |
| ART-001-g-int03 | GGAAGCUGCUGAUGACACCU | TTR (artificial) | chr18: 31592992- | 62 |
| | | | 31593007 | |
| ART-001-g-int04 | GAGAGCUGCUGAUGACACCU | TTR (artificial) | chr18: 31592992- | 63 |
| | | | 31593007 | |
| ART-001-g-int05 | AGAAGTUGCUGAUGACACCU | TTR (artificial) | chr18: 31592994- | 64 |
| | | | 31593007 | |
| ART-001-g-int06 | GAGGACUGCUGAUGACACCU | TTR (artificial) | chr18: 31592994- | 65 |
| | | | 31593007 | |
| ART-001-g-int07 | UUUUGCUGCUGAUGACACCU | TTR (artificial) | chr18: 31592992- | 66 |
| | | | 31593007 | |
| ART-001-g-int08 | CCCCGCUGCUGAUGACACCU | TTR (artificial) | chr18: 31592992- | 67 |
| | | | 31593007 | |
| ART-001-g-int09 | UUCCGCUGCUGAUGACACCU | TTR (artificial) | chr18: 31592992- | 68 |
| | | | 31593007 | |
| ART-001-g-int10 | CCUUGCUGCUGAUGACACCU | TTR (artificial) | chr18: 31592992- | 69 |
| | | | 31593007 | |
| ART-001-g-int11 | UCUCGCUGCUGAUGACACCU | TTR (artificial) | chr18: 31592992- | 70 |
| | | | 31593007 | |
| ART-001-g-int12 | CUCUGCUGCUGAUGACACCU | TTR (artificial) | chr18: 31592992- | 71 |
| | | | 31593007 | |
| ART-001-g-int13 | UUUCGCUGCUGAUGACACCU | TTR (artificial) | chr18: 31592992- | 72 |
| | | | 31593007 | |
| ART-001-g-int14 | CCCUGCUGCUGAUGACACCU | TTR (artificial) | chr18: 31592992- | 73 |
| | | | 31593007 | |
| ART-001-g-int15 | GGCCGCUGCUGAUGACACCU | TTR (artificial) | chr18: 31592992- | 74 |
| | | | 31593007 | |
| ART-001-g-int16 | GGUUGCUGCUGAUGACACCU | TTR (artificial) | chr18: 31592992- | 75 |
| | | | 31593007 | |
| ART-001-g-int17 | GCGCGCUGCUGAUGACACCU | TTR (artificial) | chr18: 31592992- | 76 |
| | | | 31593007 | |
| ART-001-g-int18 | GUGUGCUGCUGAUGACACCU | TTR (artificial) | chr18: 31592992- | 77 |
| | | | 31593007 | |
| ART-001-g-int19 | GGGCGCUGCUGAUGACACCU | TTR (artificial) | chr18: 31592992- | 78 |
| | | | 31593007 | |
| ART-001-g-int20 | GGGUGCUGCUGAUGACACCU | TTR (artificial) | chr18: 31592992- | 79 |
| | | | 31593007 | |
| ART-001-g-int21 | GCCCGCUGCUGAUGACACCU | TTR (artificial) | chr18: 31592992- | 80 |
| | | | 31593007 | |
| ART-001-g-int22 | GUUUGCUGCUGAUGACACCU | TTR (artificial) | chr18: 31592992- | 81 |
| | | | 31593007 | |
| ART-001-g-036 | AAAGUUCUAGAUGCUGUCCG | TTR (Exon 2) | chr18: 31592929- | 82 |
| (Bo-5) | | | 31592948 | |
| ART-001-g-037 | GGGCGGCAAUGGUGUAGCGG | TTR (Exon 4) | chr18: 31598600- | 83 |
| (Bo-11) | | | 31598619 | |
| ART-001-g-038 | CAUUGAUGGCAGGACUGCCU | TTR (Exon 2) | chr18: 31592949- | 84 |
| (Bo-51) | | | 31592968 | |
| ART-001-g-int | AAAGGCUGCUGAUGACACCU | TTR (Exon 2) | chr18: 31592988- | 85 |
| (NTLA-2001) | | | 31593007 | |
| ART-001-g-064 | ACACAAAUACCAGUCCAGCA | TTR (Exon 1) | chr18: 31591935- | 275 |
| | | | 31591954 | |
| ART-001-g-065 | UCUAGAACUUUGACCAUCAG | TTR (Exon 2) | chr18: 31592920- | 276 |
| | | | 31592939 | |
| ART-001-g-066 | UUACAGCCACGUCUACAGCA | mTTR (Exon 2) | | 277 |
| ART-001-g-067 | ACACAAAUACCAGUCCAGCG | mTTR (Exon 1) | | 278 |
| ART-001-g-068 | AAAGGCTGCUGAUGACACCU | TTR (Exon 2) | chr18: 31592988- | 279 |
| | | | 31593007 | |
| ART-001-g-069 | AAAGGCUGCTGAUGACACCU | TTR (Exon 2) | chr18: 31592988- | 280 |
| | | | 31593007 | |
| ART-001-g-070 | AAAGGCUGCUGATGACACCU | TTR (Exon 2) | chr18: 31592988- | 281 |
| | | | 31593007 | |
| ART-001-g-071 | AAAGGCUGCUGAUGACACCT | TTR (Exon 2) | chr18: 31592988- | 282 |
| | | | 31593007 | |
| ART-001-g-072 | AAAGGCUGCUGATGACACCT | TTR (Exon 2) | chr18: 31592988- | 283 |
| | | | 31593007 | |
| ART-001-g-073 | AAAGGCTGCTGATGACACCT | TTR (Exon 2) | chr18: 31592988- | 284 |
| | | | 31593007 | |
| ART-001-g-074 | AAGGCUGCUGAUGACACCU | TTR (Exon 1) | chr18: | 285 |
| | | | 31591950- | |
| | | | 31591969 | |
| ART-001-g-075 | AGGCUGCUGAUGACACCU | TTR (Exon 1) | chr18: | 286 |
| | | | 31591950- | |
| | | | 31591969 | |
| ART-001-g-076 | GGCUGCUGAUGACACCU | TTR (Exon 1) | chr18: | 287 |
| | | | 31591950- | |
| | | | 31591969 | |
| ART-001-g-077 | GCUGCUGAUGACACCU | TTR (Exon 1) | chr18: | 288 |
| | | | 31591950- | |
| | | | 31591969 | |
| ART-001-g-078 | AAGGCTGCTGATGACACCT | TTR (Exon 1) | chr18: | 289 |
| | | | 31591950- | |
| | | | 31591969 | |
| ART-001-g-079 | AGGCTGCTGATGACACCT | TTR (Exon 1) | chr18: | 290 |
| | | | 31591950- | |
| | | | 31591969 | |
| ART-001-g-080 | GGCTGCTGATGACACCT | TTR (Exon 1) | chr18: | 291 |
| | | | 31591950- | |
| | | | 31591969 | |
| ART-001-g-081 | GCTGCTGATGACACCT | TTR (Exon 1) | chr18: | 292 |
| | | | 31591950- | |
| | | | 31591969 | |
| ART-001-g-082 | TGUGUCUGAGGCUGGCCCUA | TTR (Exon 1) | chr18: | 293 |
| | | | 31591950- | |
| | | | 31591969 | |
| ART-001-g-083 | UGTGUCUGAGGCUGGCCCUA | TTR (Exon 1) | chr18: | 294 |
| | | | 31591950- | |
| | | | 31591969 | |
| ART-001-g-084 | UGUGTCUGAGGCUGGCCCUA | TTR (Exon 1) | chr18: | 295 |
| | | | 31591950- | |
| | | | 31591969 | |
| ART-001-g-085 | UGUGUCTGAGGCUGGCCCUA | TTR (Exon 1) | chr18: | 296 |
| | | | 31591950- | |
| | | | 31591969 | |
| ART-001-g-086 | UGUGUCUGAGGCTGGCCCUA | TTR (Exon 1) | chr18: | 297 |
| | | | 31591950- | |
| | | | 31591969 | |
| ART-001-g-087 | UGUGUCUGAGGCUGGCCCTA | TTR (Exon 1) | chr18: | 298 |
| | | | 31591950- | |
| | | | 31591969 | |
| ART-001-g-088 | TGTGTCTGAGGCUGGCCCUA | TTR (Exon 1) | chr18: | 299 |
| | | | 31591950- | |
| | | | 31591969 | |
| ART-001-g-089 | TGTGTCTGAGGCTGGCCCUA | TTR (Exon 1) | chr18: | 300 |
| | | | 31591950- | |
| | | | 31591969 | |
| ART-001-g-090 | TGTGTCTGAGGCTGGCCCTA | TTR (Exon 1) | chr18: | 301 |
| | | | 31591950- | |
| | | | 31591969 | |
| ART-001-g-091 | ACACAAATACCAGUCCAGCA | TTR (Exon 1) | chr18: 31591935- | 302 |
| | | | 31591954 | |
| ART-001-g-092 | ACACAAATACCAGTCCAGCA | TTR (Exon 1) | chr18: 31591935- | 303 |
| | | | 31591954 | |
| ART-001-g-093 | UGUGUCUfGfAfGfGCfUfGGCCCUA | TTR (Exon 1) | chr18: | 304 |
| | | | 31591950- | |
| | | | 31591969 | |
| ART-001-g-094 | CUUGGAUfUfCfAfCCGfGfUGCCCU | TTR (Exon 2) | chr18: 31592894- | 305 |
| | | | 31592913 | |
| ART-001-g-095 | ACUUGGAfUfUfCfACCfGfGUGCCC | TTR (Exon 2) | chr18: 31592895- | 306 |
| | | | 31592914 | |
| ART-001-g-096 | AAAGGCUfGfCfUfGAfUfGACACCU | TTR (Exon 2) | chr18: 31592988- | 307 |
| | | | 31593007 | |

Note:
the character “f” within a polynucleotide sequence disclosed herein indicates a 2′ fluoro base, wherein the base has a fluorine modified ribose. The “f” indicates fluoro modification of the base that follows the “f” in the polynucleotide sequence, e.g., CUfGATU comprises a 2′ fluoro G.

TABLE 4
crRNA and sgRNA sequences corresponding to the guide sequences in Table 3

| | Guide | | | | |
|---|---|---|---|---|---|
| | sequence | | crRNA | | sgRNA |
| | SEQ ID | | SEQ ID | | SEQ ID |
| Name | NO: | crRNA sequence | NO: | sgRNA Sequence | NO: |
| ART- | 1 | CCCAUUCCAUGAGC | 86 | CCCAUUCCAUGAGCAUGCAGGUUUUA | 171 |
| 001-g- | | AUGCAGGUUUUAGA | | GAGCUAGAAAUAGCAAGUUAAAAUAA | |
| 006 | | GCUAUGCUGUUUUG | | GGCUAGUCCGUUAUCAACUUGAAAAA | |
| | | | | GUGGCACCGAGUCGGUGCUUUU | |
| ART- | 2 | GCCAUCCUGCCAAG | 87 | GCCAUCCUGCCAAGAAUGAGGUUUUA | 172 |
| 001-g- | | AAUGAGGUUUUAGA | | GAGCUAGAAAUAGCAAGUUAAAAUAA | |
| 012 | | GCUAUGCUGUUUUG | | GGCUAGUCCGUUAUCAACUUGAAAAA | |
| | | | | GUGGCACCGAGUCGGUGCUUUU | |
| ART- | 3 | GAAUGAGUGGACUU | 88 | GAAUGAGUGGACUUCUGUGAGUUUUA | 173 |
| 001-g- | | CUGUGAGUUUUAGA | | GAGCUAGAAAUAGCAAGUUAAAAUAA | |
| 015 | | GCUAUGCUGUUUUG | | GGCUAGUCCGUUAUCAACUUGAAAAA | |
| | | | | GUGGCACCGAGUCGGUGCUUUU | |
| ART- | 4 | UCCUAUAAGGUGUG | 89 | UCCUAUAAGGUGUGAAAGUCGUUUUA | 174 |
| 001-g- | | AAAGUCGUUUUAGA | | GAGCUAGAAAUAGCAAGUUAAAAUAA | |
| 017 | | GCUAUGCUGUUUUG | | GGCUAGUCCGUUAUCAACUUGAAAAA | |
| | | | | GUGGCACCGAGUCGGUGCUUUU | |
| ART- | 5 | GUGAGUAUACAGAC | 90 | GUGAGUAUACAGACCUUCGAGUUUUA | 175 |
| 001-g- | | CUUCGAGUUUUAGA | | GAGCUAGAAAUAGCAAGUUAAAAUAA | |
| 018 | | GCUAUGCUGUUUUG | | GGCUAGUCCGUUAUCAACUUGAAAAA | |
| | | | | GUGGCACCGAGUCGGUGCUUUU | |
| ART- | 6 | GGUGAGUAUACAGA | 91 | GGUGAGUAUACAGACCUUCGGUUUUA | 176 |
| 001-g- | | CCUUCGGUUUUAGA | | GAGCUAGAAAUAGCAAGUUAAAAUAA | |
| 019 | | GCUAUGCUGUUUUG | | GGCUAGUCCGUUAUCAACUUGAAAAA | |
| | | | | GUGGCACCGAGUCGGUGCUUUU | |
| ART- | 7 | AUGAGGGACUUCUC | 92 | AUGAGGGACUUCUCCUCCAGGUUUUA | 177 |
| 001-g- | | CUCCAGGUUUUAGA | | GAGCUAGAAAUAGCAAGUUAAAAUAA | |
| 020 | | GCUAUGCUGUUUUG | | GGCUAGUCCGUUAUCAACUUGAAAAA | |
| | | | | GUGGCACCGAGUCGGUGCUUUU | |
| ART- | 8 | UUCUCCUCCAGUGG | 93 | UUCUCCUCCAGUGGACCUGAGUUUUA | 178 |
| 001-g- | | ACCUGAGUUUUAGA | | GAGCUAGAAAUAGCAAGUUAAAAUAA | |
| 021 | | GCUAUGCUGUUUUG | | GGCUAGUCCGUUAUCAACUUGAAAAA | |
| | | | | GUGGCACCGAGUCGGUGCUUUU | |
| ART- | 9 | CCAGUGGACCUGAA | 94 | CCAGUGGACCUGAAGGACGAGUUUUA | 179 |
| 001-g- | | GGACGAGUUUUAGA | | GAGCUAGAAAUAGCAAGUUAAAAUAA | |
| 022 | | GCUAUGCUGUUUUG | | GGCUAGUCCGUUAUCAACUUGAAAAA | |
| | | | | GUGGCACCGAGUCGGUGCUUUU | |
| ART- | 10 | UCCAGUGGACCUGA | 95 | UCCAGUGGACCUGAAGGACGGUUUUA | 180 |
| 001-g- | | AGGACGGUUUUAGA | | GAGCUAGAAAUAGCAAGUUAAAAUAA | |
| 023 | | GCUAUGCUGUUUUG | | GGCUAGUCCGUUAUCAACUUGAAAAA | |
| | | | | GUGGCACCGAGUCGGUGCUUUU | |
| ART- | 11 | UCGUCCUUCAGGUC | 96 | UCGUCCUUCAGGUCCACUGGGUUUUA | 181 |
| 001-g- | | CACUGGGUUUUAGA | | GAGCUAGAAAUAGCAAGUUAAAAUAA | |
| 024 | | GCUAUGCUGUUUUG | | GGCUAGUCCGUUAUCAACUUGAAAAA | |
| | | | | GUGGCACCGAGUCGGUGCUUUU | |
| ART- | 12 | AAUCCCAUCCCUCG | 97 | AAUCCCAUCCCUCGUCCUUCGUUUUA | 182 |
| 001-g- | | UCCUUCGUUUUAGA | | GAGCUAGAAAUAGCAAGUUAAAAUAA | |
| 025 | | GCUAUGCUGUUUUG | | GGCUAGUCCGUUAUCAACUUGAAAAA | |
| | | | | GUGGCACCGAGUCGGUGCUUUU | |
| ART- | 13 | CCCUCGUCCUUCAG | 98 | CCCUCGUCCUUCAGGUCCACGUUUUA | 183 |
| 001-g- | | GUCCACGUUUUAGA | | GAGCUAGAAAUAGCAAGUUAAAAUAA | |
| 026 | | GCUAUGCUGUUUUG | | GGCUAGUCCGUUAUCAACUUGAAAAA | |
| | | | | GUGGCACCGAGUCGGUGCUUUU | |
| ART- | 14 | UGGACCUGAAGGAC | 99 | UGGACCUGAAGGACGAGGGAGUUUUA | 184 |
| 001-g- | | GAGGGAGUUUUAGA | | GAGCUAGAAAUAGCAAGUUAAAAUAA | |
| 027 | | GCUAUGCUGUUUUG | | GGCUAGUCCGUUAUCAACUUGAAAAA | |
| | | | | GUGGCACCGAGUCGGUGCUUUU | |
| ART- | 15 | GGACCUGAAGGACG | 100 | GGACCUGAAGGACGAGGGAUGUUUUA | 185 |
| 001-g- | | AGGGAUGUUUUAGA | | GAGCUAGAAAUAGCAAGUUAAAAUAA | |
| 028 | | GCUAUGCUGUUUUG | | GGCUAGUCCGUUAUCAACUUGAAAAA | |
| | | | | GUGGCACCGAGUCGGUGCUUUU | |
| ART- | 16 | UAGUAAAAAUGGAA | 101 | UAGUAAAAAUGGAAUACUCUGUUUUA | 186 |
| 001-g- | | UACUCUGUUUUAGA | | GAGCUAGAAAUAGCAAGUUAAAAUAA | |
| 029 | | GCUAUGCUGUUUUG | | GGCUAGUCCGUUAUCAACUUGAAAAA | |
| | | | | GUGGCACCGAGUCGGUGCUUUU | |
| ART- | 17 | AAACACUGCUUUAG | 102 | AAACACUGCUUUAGUAAAAAGUUUUA | 187 |
| 001-g- | | UAAAAAGUUUUAGA | | GAGCUAGAAAUAGCAAGUUAAAAUAA | |
| 030 | | GCUAUGCUGUUUUG | | GGCUAGUCCGUUAUCAACUUGAAAAA | |
| | | | | GUGGCACCGAGUCGGUGCUUUU | |
| ART- | 18 | GACUUCUAACAUAG | 103 | GACUUCUAACAUAGCAUAUGGUUUUA | 188 |
| 001-g- | | CAUAUGGUUUUAGA | | GAGCUAGAAAUAGCAAGUUAAAAUAA | |
| 031 | | GCUAUGCUGUUUUG | | GGCUAGUCCGUUAUCAACUUGAAAAA | |
| | | | | GUGGCACCGAGUCGGUGCUUUU | |
| ART- | 19 | AUAUGCUAUGUUAG | 104 | AUAUGCUAUGUUAGAAGUCCGUUUUA | 189 |
| 001-g- | | AAGUCCGUUUUAGA | | GAGCUAGAAAUAGCAAGUUAAAAUAA | |
| 032 | | GCUAUGCUGUUUUG | | GGCUAGUCCGUUAUCAACUUGAAAAA | |
| | | | | GUGGCACCGAGUCGGUGCUUUU | |
| ART- | 20 | AAUGUUUUAUUGUC | 105 | AAUGUUUUAUUGUCUCUGCCGUUUUA | 190 |
| 001-g- | | UCUGCCGUUUUAGA | | GAGCUAGAAAUAGCAAGUUAAAAUAA | |
| 033 | | GCUAUGCUGUUUUG | | GGCUAGUCCGUUAUCAACUUGAAAAA | |
| | | | | GUGGCACCGAGUCGGUGCUUUU | |
| ART- | 21 | CAAUAAAACAUUCC | 106 | CAAUAAAACAUUCCUGUGAAGUUUUA | 191 |
| 001-g- | | UGUGAAGUUUUAGA | | GAGCUAGAAAUAGCAAGUUAAAAUAA | |
| 034 | | GCUAUGCUGUUUUG | | GGCUAGUCCGUUAUCAACUUGAAAAA | |
| | | | | GUGGCACCGAGUCGGUGCUUUU | |
| ART- | 22 | GAAUGAAAAGUGCC | 107 | GAAUGAAAAGUGCCUUUCACGUUUUA | 192 |
| 001-g- | | UUUCACGUUUUAGA | | GAGCUAGAAAUAGCAAGUUAAAAUAA | |
| 035 | | GCUAUGCUGUUUUG | | GGCUAGUCCGUUAUCAACUUGAAAAA | |
| | | | | GUGGCACCGAGUCGGUGCUUUU | |
| ART- | 23 | AGUCAAUAAUCAGA | 108 | AGUCAAUAAUCAGAAUCAGCGUUUUA | 193 |
| 001-g- | | AUCAGCGUUUUAGA | | GAGCUAGAAAUAGCAAGUUAAAAUAA | |
| 039 | | GCUAUGCUGUUUUG | | GGCUAGUCCGUUAUCAACUUGAAAAA | |
| | | | | GUGGCACCGAGUCGGUGCUUUU | |
| ART- | 24 | CAGCAGGUUUGCAG | 109 | CAGCAGGUUUGCAGUCAGAUGUUUUA | 194 |
| 001-g- | | UCAGAUGUUUUAGA | | GAGCUAGAAAUAGCAAGUUAAAAUAA | |
| 040 | | GCUAUGCUGUUUUG | | GGCUAGUCCGUUAUCAACUUGAAAAA | |
| | | | | GUGGCACCGAGUCGGUGCUUUU | |
| ART- | 25 | AGGUUUGCAGUCAG | 110 | AGGUUUGCAGUCAGAUUGGCGUUUUA | 195 |
| 001-g- | | AUUGGCGUUUUAGA | | GAGCUAGAAAUAGCAAGUUAAAAUAA | |
| 041 | | GCUAUGCUGUUUUG | | GGCUAGUCCGUUAUCAACUUGAAAAA | |
| | | | | GUGGCACCGAGUCGGUGCUUUU | |
| ART- | 26 | GGUUUGCAGUCAGA | 111 | GGUUUGCAGUCAGAUUGGCAGUUUUA | 196 |
| 001-g- | | UUGGCAGUUUUAGA | | GAGCUAGAAAUAGCAAGUUAAAAUAA | |
| 042 | | GCUAUGCUGUUUUG | | GGCUAGUCCGUUAUCAACUUGAAAAA | |
| | | | | GUGGCACCGAGUCGGUGCUUUU | |
| ART- | 27 | AGGGAUAAGCAGCC | 112 | AGGGAUAAGCAGCCUAGCUCGUUUUA | 197 |
| 001-g- | | UAGCUCGUUUUAGA | | GAGCUAGAAAUAGCAAGUUAAAAUAA | |
| 043 | | GCUAUGCUGUUUUG | | GGCUAGUCCGUUAUCAACUUGAAAAA | |
| | | | | GUGGCACCGAGUCGGUGCUUUU | |
| ART- | 28 | AUACUCACUUCUCC | 113 | AUACUCACUUCUCCUGAGCUGUUUUA | 198 |
| 001-g- | | UGAGCUGUUUUAGA | | GAGCUAGAAAUAGCAAGUUAAAAUAA | |
| 044 | | GCUAUGCUGUUUUG | | GGCUAGUCCGUUAUCAACUUGAAAAA | |
| | | | | GUGGCACCGAGUCGGUGCUUUU | |
| ART- | 29 | GAAGUGAGUAUAAA | 114 | GAAGUGAGUAUAAAAGCCCCGUUUUA | 199 |
| 001-g- | | AGCCCCGUUUUAGA | | GAGCUAGAAAUAGCAAGUUAAAAUAA | |
| 045 | | GCUAUGCUGUUUUG | | GGCUAGUCCGUUAUCAACUUGAAAAA | |
| | | | | GUGGCACCGAGUCGGUGCUUUU | |
| ART- | 30 | UGAGUAUAAAAGCC | 115 | UGAGUAUAAAAGCCCCAGGCGUUUUA | 200 |
| 001-g- | | CCAGGCGUUUUAGA | | GAGCUAGAAAUAGCAAGUUAAAAUAA | |
| 046 | | GCUAUGCUGUUUUG | | GGCUAGUCCGUUAUCAACUUGAAAAA | |
| | | | | GUGGCACCGAGUCGGUGCUUUU | |
| ART- | 31 | GAGUAUAAAAGCCC | 116 | GAGUAUAAAAGCCCCAGGCUGUUUUA | 201 |
| 001-g- | | CAGGCUGUUUUAGA | | GAGCUAGAAAUAGCAAGUUAAAAUAA | |
| 047 | | GCUAUGCUGUUUUG | | GGCUAGUCCGUUAUCAACUUGAAAAA | |
| | | | | GUGGCACCGAGUCGGUGCUUUU | |
| ART- | 32 | UGAUGGCUGCUCCC | 117 | UGAUGGCUGCUCCCAGCCUGGUUUUA | 202 |
| 001-g- | | AGCCUGGUUUUAGA | | GAGCUAGAAAUAGCAAGUUAAAAUAA | |
| 048 | | GCUAUGCUGUUUUG | | GGCUAGUCCGUUAUCAACUUGAAAAA | |
| | | | | GUGGCACCGAGUCGGUGCUUUU | |
| ART- | 33 | GUGAUGGCUGCUCC | 118 | GUGAUGGCUGCUCCCAGCCUGUUUUA | 203 |
| 001-g- | | CAGCCUGUUUUAGA | | GAGCUAGAAAUAGCAAGUUAAAAUAA | |
| 049 | | GCUAUGCUGUUUUG | | GGCUAGUCCGUUAUCAACUUGAAAAA | |
| | | | | GUGGCACCGAGUCGGUGCUUUU | |
| ART- | 34 | UGUGAUGGCUGCUC | 119 | UGUGAUGGCUGCUCCCAGCCGUUUUA | 204 |
| 001-g- | | CCAGCCGUUUUAGA | | GAGCUAGAAAUAGCAAGUUAAAAUAA | |
| 050 | | GCUAUGCUGUUUUG | | GGCUAGUCCGUUAUCAACUUGAAAAA | |
| | | | | GUGGCACCGAGUCGGUGCUUUU | |
| ART- | 35 | UUUAAAAAAUCAAG | 120 | UUUAAAAAAUCAAGUUAAAGGUUUUA | 205 |
| 001-g- | | UUAAAGGUUUUAGA | | GAGCUAGAAAUAGCAAGUUAAAAUAA | |
| 051 | | GCUAUGCUGUUUUG | | GGCUAGUCCGUUAUCAACUUGAAAAA | |
| | | | | GUGGCACCGAGUCGGUGCUUUU | |
| ART- | 36 | UUUUUUGGAAGGGA | 121 | UUUUUUGGAAGGGACAAUAAGUUUUA | 206 |
| 001-g- | | CAAUAAGUUUUAGA | | GAGCUAGAAAUAGCAAGUUAAAAUAA | |
| 052 | | GCUAUGCUGUUUUG | | GGCUAGUCCGUUAUCAACUUGAAAAA | |
| | | | | GUGGCACCGAGUCGGUGCUUUU | |
| ART- | 37 | UUUUUUUGGAAGGG | 122 | UUUUUUUGGAAGGGACAAUAGUUUUA | 207 |
| 001-g- | | ACAAUAGUUUUAGA | | GAGCUAGAAAUAGCAAGUUAAAAUAA | |
| 053 | | GCUAUGCUGUUUUG | | GGCUAGUCCGUUAUCAACUUGAAAAA | |
| | | | | GUGGCACCGAGUCGGUGCUUUU | |
| ART- | 38 | UGAUUCUCUUUUUU | 123 | UGAUUCUCUUUUUUUUGGAAGUUUUA | 208 |
| 001-g- | | UUGGAAGUUUUAGA | | GAGCUAGAAAUAGCAAGUUAAAAUAA | |
| 054 | | GCUAUGCUGUUUUG | | GGCUAGUCCGUUAUCAACUUGAAAAA | |
| | | | | GUGGCACCGAGUCGGUGCUUUU | |
| ART- | 39 | UUGAUUCUCUUUUU | 124 | UUGAUUCUCUUUUUUUUGGAGUUUUA | 209 |
| 001-g- | | UUUGGAGUUUUAGA | | GAGCUAGAAAUAGCAAGUUAAAAUAA | |
| 055 | | GCUAUGCUGUUUUG | | GGCUAGUCCGUUAUCAACUUGAAAAA | |
| | | | | GUGGCACCGAGUCGGUGCUUUU | |
| ART- | 40 | AAUUUUGAUUCUCU | 125 | AAUUUUGAUUCUCUUUUUUUGUUUUA | 210 |
| 001-g- | | UUUUUUGUUUUAGA | | GAGCUAGAAAUAGCAAGUUAAAAUAA | |
| 056 | | GCUAUGCUGUUUUG | | GGCUAGUCCGUUAUCAACUUGAAAAA | |
| | | | | GUGGCACCGAGUCGGUGCUUUU | |
| ART- | 41 | AAAAUUUUACAAAG | 126 | AAAAUUUUACAAAGAAUCAAGUUUUA | 211 |
| 001-g- | | AAUCAAGUUUUAGA | | GAGCUAGAAAUAGCAAGUUAAAAUAA | |
| 057 | | GCUAUGCUGUUUUG | | GGCUAGUCCGUUAUCAACUUGAAAAA | |
| | | | | GUGGCACCGAGUCGGUGCUUUU | |
| ART- | 42 | AAGGAAUUCUAGAA | 127 | AAGGAAUUCUAGAAAGUAUCGUUUUA | 212 |
| 001-g- | | AGUAUCGUUUUAGA | | GAGCUAGAAAUAGCAAGUUAAAAUAA | |
| 058 | | GCUAUGCUGUUUUG | | GGCUAGUCCGUUAUCAACUUGAAAAA | |
| | | | | GUGGCACCGAGUCGGUGCUUUU | |
| ART- | 43 | AGGAAUUCUAGAAA | 128 | AGGAAUUCUAGAAAGUAUCUGUUUUA | 213 |
| 001-g- | | GUAUCUGUUUUAGA | | GAGCUAGAAAUAGCAAGUUAAAAUAA | |
| 059 | | GCUAUGCUGUUUUG | | GGCUAGUCCGUUAUCAACUUGAAAAA | |
| | | | | GUGGCACCGAGUCGGUGCUUUU | |
| ART- | 44 | AAGUAUCUGGGCAG | 129 | AAGUAUCUGGGCAGAACGCUGUUUUA | 214 |
| 001-g- | | AACGCUGUUUUAGA | | GAGCUAGAAAUAGCAAGUUAAAAUAA | |
| 060 | | GCUAUGCUGUUUUG | | GGCUAGUCCGUUAUCAACUUGAAAAA | |
| | | | | GUGGCACCGAGUCGGUGCUUUU | |
| ART- | 45 | CUUGCAAGACAAUG | 130 | CUUGCAAGACAAUGGAAAUUGUUUUA | 215 |
| 001-g- | | GAAAUUGUUUUAGA | | GAGCUAGAAAUAGCAAGUUAAAAUAA | |
| 061 | | GCUAUGCUGUUUUG | | GGCUAGUCCGUUAUCAACUUGAAAAA | |
| | | | | GUGGCACCGAGUCGGUGCUUUU | |
| ART- | 46 | GUGCUUUGCUUGCA | 131 | GUGCUUUGCUUGCAAGACAAGUUUUA | 216 |
| 001-g- | | AGACAAGUUUUAGA | | GAGCUAGAAAUAGCAAGUUAAAAUAA | |
| 062 | | GCUAUGCUGUUUUG | | GGCUAGUCCGUUAUCAACUUGAAAAA | |
| | | | | GUGGCACCGAGUCGGUGCUUUU | |
| ART- | 47 | UACAGAAUGUGUCU | 132 | UACAGAAUGUGUCUUUUUAAGUUUUA | 217 |
| 001-g- | | UUUUAAGUUUUAGA | | GAGCUAGAAAUAGCAAGUUAAAAUAA | |
| 063 | | GCUAUGCUGUUUUG | | GGCUAGUCCGUUAUCAACUUGAAAAA | |
| | | | | GUGGCACCGAGUCGGUGCUUUU | |
| ART- | 48 | UGUGUCUGAGGCUG | 133 | UGUGUCUGAGGCUGGCCCUAGUUUUA | 218 |
| 001-g- | | GCCCUAGUUUUAGA | | GAGCUAGAAAUAGCAAGUUAAAAUAA | |
| 001 | | GCUAUGCUGUUUUG | | GGCUAGUCCGUUAUCAACUUGAAAAA | |
| | | | | GUGGCACCGAGUCGGUGCUUUU | |
| ART- | 49 | CUUGGAUUCACCGG | 134 | CUUGGAUUCACCGGUGCCCUGUUUUA | 219 |
| 001-g- | | UGCCCUGUUUUAGA | | GAGCUAGAAAUAGCAAGUUAAAAUAA | |
| 002 | | GCUAUGCUGUUUUG | | GGCUAGUCCGUUAUCAACUUGAAAAA | |
| | | | | GUGGCACCGAGUCGGUGCUUUU | |
| ART- | 50 | ACUUGGAUUCACCG | 135 | ACUUGGAUUCACCGGUGCCCGUUUUA | 220 |
| 001-g- | | GUGCCCGUUUUAGA | | GAGCUAGAAAUAGCAAGUUAAAAUAA | |
| 003 | | GCUAUGCUGUUUUG | | GGCUAGUCCGUUAUCAACUUGAAAAA | |
| | | | | GUGGCACCGAGUCGGUGCUUUU | |
| ART- | 51 | UCCAGACUUUCACA | 136 | UCCAGACUUUCACACCUUAUGUUUUA | 221 |
| 001-g- | | CCUUAUGUUUUAGA | | GAGCUAGAAAUAGCAAGUUAAAAUAA | |
| 004 | | GCUAUGCUGUUUUG | | GGCUAGUCCGUUAUCAACUUGAAAAA | |
| | | | | GUGGCACCGAGUCGGUGCUUUU | |
| ART- | 52 | GACUCACUGGUUUU | 137 | GACUCACUGGUUUUCCUAUAGUUUUA | 222 |
| 001-g- | | CCUAUAGUUUUAGA | | GAGCUAGAAAUAGCAAGUUAAAAUAA | |
| 005 | | GCUAUGCUGUUUUG | | GGCUAGUCCGUUAUCAACUUGAAAAA | |
| | | | | GUGGCACCGAGUCGGUGCUUUU | |
| ART- | 53 | AUCUGUCUGUCUUC | 138 | AUCUGUCUGUCUUCUCUCAUGUUUUA | 223 |
| 001-g- | | UCUCAUGUUUUAGA | | GAGCUAGAAAUAGCAAGUUAAAAUAA | |
| 007 | | GCUAUGCUGUUUUG | | GGCUAGUCCGUUAUCAACUUGAAAAA | |
| | | | | GUGGCACCGAGUCGGUGCUUUU | |
| ART- | 54 | GUCACCAAUCCCAA | 139 | GUCACCAAUCCCAAGGAAUGGUUUUA | 224 |
| 001-g- | | GGAAUGGUUUUAGA | | GAGCUAGAAAUAGCAAGUUAAAAUAA | |
| 008 | | GCUAUGCUGUUUUG | | GGCUAGUCCGUUAUCAACUUGAAAAA | |
| | | | | GUGGCACCGAGUCGGUGCUUUU | |
| ART- | 55 | UCACCAAUCCCAAG | 140 | UCACCAAUCCCAAGGAAUGAGUUUUA | 225 |
| 001-g- | | GAAUGAGUUUUAGA | | GAGCUAGAAAUAGCAAGUUAAAAUAA | |
| 009 | | GCUAUGCUGUUUUG | | GGCUAGUCCGUUAUCAACUUGAAAAA | |
| | | | | GUGGCACCGAGUCGGUGCUUUU | |
| ART- | 56 | AGGAGAAGUCCCUC | 141 | AGGAGAAGUCCCUCAUUCCUGUUUUA | 226 |
| 001-g- | | AUUCCUGUUUUAGA | | GAGCUAGAAAUAGCAAGUUAAAAUAA | |
| 010 | | GCUAUGCUGUUUUG | | GGCUAGUCCGUUAUCAACUUGAAAAA | |
| | | | | GUGGCACCGAGUCGGUGCUUUU | |
| ART- | 57 | GGAGAAGUCCCUCA | 142 | GGAGAAGUCCCUCAUUCCUUGUUUUA | 227 |
| 001-g- | | UUCCUUGUUUUAGA | | GAGCUAGAAAUAGCAAGUUAAAAUAA | |
| 011 | | GCUAUGCUGUUUUG | | GGCUAGUCCGUUAUCAACUUGAAAAA | |
| | | | | GUGGCACCGAGUCGGUGCUUUU | |
| ART- | 58 | GAAGUCCACUCAUU | 143 | GAAGUCCACUCAUUCUUGGCGUUUUA | 228 |
| 001-g- | | CUUGGCGUUUUAGA | | GAGCUAGAAAUAGCAAGUUAAAAUAA | |
| 013 | | GCUAUGCUGUUUUG | | GGCUAGUCCGUUAUCAACUUGAAAAA | |
| | | | | GUGGCACCGAGUCGGUGCUUUU | |
| ART- | 59 | CACAGAAGUCCACU | 144 | CACAGAAGUCCACUCAUUCUGUUUUA | 229 |
| 001-g- | | CAUUCUGUUUUAGA | | GAGCUAGAAAUAGCAAGUUAAAAUAA | |
| 014 | | GCUAUGCUGUUUUG | | GGCUAGUCCGUUAUCAACUUGAAAAA | |
| | | | | GUGGCACCGAGUCGGUGCUUUU | |
| ART- | 60 | GGGAGCUGCUGAUG | 145 | GGGAGCUGCUGAUGACACCUGUUUUA | 230 |
| 001-g- | | ACACCUGUUUUAGA | | GAGCUAGAAAUAGCAAGUUAAAAUAA | |
| int01 | | GCUAUGCUGUUUUG | | GGCUAGUCCGUUAUCAACUUGAAAAA | |
| | | | | GUGGCACCGAGUCGGUGCUUUU | |
| ART- | 61 | AGGAGCUGCUGAUG | 146 | AGGAGCUGCUGAUGACACCUGUUUUA | 231 |
| 001-g- | | ACACCUGUUUUAGA | | GAGCUAGAAAUAGCAAGUUAAAAUAA | |
| int02 | | GCUAUGCUGUUUUG | | GGCUAGUCCGUUAUCAACUUGAAAAA | |
| | | | | GUGGCACCGAGUCGGUGCUUUU | |
| ART- | 62 | GGAAGCUGCUGAUG | 147 | GGAAGCUGCUGAUGACACCUGUUUUA | 232 |
| 001-g- | | ACACCUGUUUUAGA | | GAGCUAGAAAUAGCAAGUUAAAAUAA | |
| int03 | | GCUAUGCUGUUUUG | | GGCUAGUCCGUUAUCAACUUGAAAAA | |
| | | | | GUGGCACCGAGUCGGUGCUUUU | |
| ART- | 63 | GAGAGCUGCUGAUG | 148 | GAGAGCUGCUGAUGACACCUGUUUUA | 233 |
| 001-g- | | ACACCUGUUUUAGA | | GAGCUAGAAAUAGCAAGUUAAAAUAA | |
| int04 | | GCUAUGCUGUUUUG | | GGCUAGUCCGUUAUCAACUUGAAAAA | |
| | | | | GUGGCACCGAGUCGGUGCUUUU | |
| ART- | 64 | AGAAGTUGCUGAUG | 149 | AGAAGTUGCUGAUGACACCUGUUUUA | 234 |
| 001-g- | | ACACCUGUUUUAGA | | GAGCUAGAAAUAGCAAGUUAAAAUAA | |
| int05 | | GCUAUGCUGUUUUG | | GGCUAGUCCGUUAUCAACUUGAAAAA | |
| | | | | GUGGCACCGAGUCGGUGCUUUU | |
| ART- | 65 | GAGGACUGCUGAUG | 150 | GAGGACUGCUGAUGACACCUGUUUUA | 235 |
| 001-g- | | ACACCUGUUUUAGA | | GAGCUAGAAAUAGCAAGUUAAAAUAA | |
| int06 | | GCUAUGCUGUUUUG | | GGCUAGUCCGUUAUCAACUUGAAAAA | |
| | | | | GUGGCACCGAGUCGGUGCUUUU | |
| ART- | 66 | UUUUGCUGCUGAUG | 151 | UUUUGCUGCUGAUGACACCUGUUUUA | 236 |
| 001-g- | | ACACCUGUUUUAGA | | GAGCUAGAAAUAGCAAGUUAAAAUAA | |
| int07 | | GCUAUGCUGUUUUG | | GGCUAGUCCGUUAUCAACUUGAAAAA | |
| | | | | GUGGCACCGAGUCGGUGCUUUU | |
| ART- | 67 | CCCCGCUGCUGAUG | 152 | CCCCGCUGCUGAUGACACCUGUUUUA | 237 |
| 001-g- | | ACACCUGUUUUAGA | | GAGCUAGAAAUAGCAAGUUAAAAUAA | |
| int08 | | GCUAUGCUGUUUUG | | GGCUAGUCCGUUAUCAACUUGAAAAA | |
| | | | | GUGGCACCGAGUCGGUGCUUUU | |
| ART- | 68 | UUCCGCUGCUGAUG | 153 | UUCCGCUGCUGAUGACACCUGUUUUA | 238 |
| 001-g- | | ACACCUGUUUUAGA | | GAGCUAGAAAUAGCAAGUUAAAAUAA | |
| int09 | | GCUAUGCUGUUUUG | | GGCUAGUCCGUUAUCAACUUGAAAAA | |
| | | | | GUGGCACCGAGUCGGUGCUUUU | |
| ART- | 69 | CCUUGCUGCUGAUG | 154 | CCUUGCUGCUGAUGACACCUGUUUUA | 239 |
| 001-g- | | ACACCUGUUUUAGA | | GAGCUAGAAAUAGCAAGUUAAAAUAA | |
| int10 | | GCUAUGCUGUUUUG | | GGCUAGUCCGUUAUCAACUUGAAAAA | |
| | | | | GUGGCACCGAGUCGGUGCUUUU | |
| ART- | 70 | UCUCGCUGCUGAUG | 155 | UCUCGCUGCUGAUGACACCUGUUUUA | 240 |
| 001-g- | | ACACCUGUUUUAGA | | GAGCUAGAAAUAGCAAGUUAAAAUAA | |
| int11 | | GCUAUGCUGUUUUG | | GGCUAGUCCGUUAUCAACUUGAAAAA | |
| | | | | GUGGCACCGAGUCGGUGCUUUU | |
| ART- | 71 | CUCUGCUGCUGAUG | 156 | CUCUGCUGCUGAUGACACCUGUUUUA | 241 |
| 001-g- | | ACACCUGUUUUAGA | | GAGCUAGAAAUAGCAAGUUAAAAUAA | |
| int12 | | GCUAUGCUGUUUUG | | GGCUAGUCCGUUAUCAACUUGAAAAA | |
| | | | | GUGGCACCGAGUCGGUGCUUUU | |
| ART- | 72 | UUUCGCUGCUGAUG | 157 | UUUCGCUGCUGAUGACACCUGUUUUA | 242 |
| 001-g- | | ACACCUGUUUUAGA | | GAGCUAGAAAUAGCAAGUUAAAAUAA | |
| int13 | | GCUAUGCUGUUUUG | | GGCUAGUCCGUUAUCAACUUGAAAAA | |
| | | | | GUGGCACCGAGUCGGUGCUUUU | |
| ART- | 73 | CCCUGCUGCUGAUG | 158 | CCCUGCUGCUGAUGACACCUGUUUUA | 243 |
| 001-g- | | ACACCUGUUUUAGA | | GAGCUAGAAAUAGCAAGUUAAAAUAA | |
| int14 | | GCUAUGCUGUUUUG | | GGCUAGUCCGUUAUCAACUUGAAAAA | |
| | | | | GUGGCACCGAGUCGGUGCUUUU | |
| ART- | 74 | GGCCGCUGCUGAUG | 159 | GGCCGCUGCUGAUGACACCUGUUUUA | 244 |
| 001-g- | | ACACCUGUUUUAGA | | GAGCUAGAAAUAGCAAGUUAAAAUAA | |
| int15 | | GCUAUGCUGUUUUG | | GGCUAGUCCGUUAUCAACUUGAAAAA | |
| | | | | GUGGCACCGAGUCGGUGCUUUU | |
| ART- | 75 | GGUUGCUGCUGAUG | 160 | GGUUGCUGCUGAUGACACCUGUUUUA | 245 |
| 001-g- | | ACACCUGUUUUAGA | | GAGCUAGAAAUAGCAAGUUAAAAUAA | |
| int16 | | GCUAUGCUGUUUUG | | GGCUAGUCCGUUAUCAACUUGAAAAA | |
| | | | | GUGGCACCGAGUCGGUGCUUUU | |
| ART- | 76 | GCGCGCUGCUGAUG | 161 | GCGCGCUGCUGAUGACACCUGUUUUA | 246 |
| 001-g- | | ACACCUGUUUUAGA | | GAGCUAGAAAUAGCAAGUUAAAAUAA | |
| int17 | | GCUAUGCUGUUUUG | | GGCUAGUCCGUUAUCAACUUGAAAAA | |
| | | | | GUGGCACCGAGUCGGUGCUUUU | |
| ART- | 77 | GUGUGCUGCUGAUG | 162 | GUGUGCUGCUGAUGACACCUGUUUUA | 247 |
| 001-g- | | ACACCUGUUUUAGA | | GAGCUAGAAAUAGCAAGUUAAAAUAA | |
| int18 | | GCUAUGCUGUUUUG | | GGCUAGUCCGUUAUCAACUUGAAAAA | |
| | | | | GUGGCACCGAGUCGGUGCUUUU | |
| ART- | 78 | GGGCGCUGCUGAUG | 163 | GGGCGCUGCUGAUGACACCUGUUUUA | 248 |
| 001-g- | | ACACCUGUUUUAGA | | GAGCUAGAAAUAGCAAGUUAAAAUAA | |
| int19 | | GCUAUGCUGUUUUG | | GGCUAGUCCGUUAUCAACUUGAAAAA | |
| | | | | GUGGCACCGAGUCGGUGCUUUU | |
| ART- | 79 | GGGUGCUGCUGAUG | 164 | GGGUGCUGCUGAUGACACCUGUUUUA | 249 |
| 001-g- | | ACACCUGUUUUAGA | | GAGCUAGAAAUAGCAAGUUAAAAUAA | |
| int20 | | GCUAUGCUGUUUUG | | GGCUAGUCCGUUAUCAACUUGAAAAA | |
| | | | | GUGGCACCGAGUCGGUGCUUUU | |
| ART- | 80 | GCCCGCUGCUGAUG | 165 | GCCCGCUGCUGAUGACACCUGUUUUA | 250 |
| 001-g- | | ACACCUGUUUUAGA | | GAGCUAGAAAUAGCAAGUUAAAAUAA | |
| int21 | | GCUAUGCUGUUUUG | | GGCUAGUCCGUUAUCAACUUGAAAAA | |
| | | | | GUGGCACCGAGUCGGUGCUUUU | |
| ART- | 81 | GUUUGCUGCUGAUG | 166 | GUUUGCUGCUGAUGACACCUGUUUUA | 251 |
| 001-g- | | ACACCUGUUUUAGA | | GAGCUAGAAAUAGCAAGUUAAAAUAA | |
| int22 | | GCUAUGCUGUUUUG | | GGCUAGUCCGUUAUCAACUUGAAAAA | |
| | | | | GUGGCACCGAGUCGGUGCUUUU | |
| ART- | 82 | AAAGUUCUAGAUGC | 167 | AAAGUUCUAGAUGCUGUCCGGUUUUA | 252 |
| 001-g- | | UGUCCGGUUUUAGA | | GAGCUAGAAAUAGCAAGUUAAAAUAA | |
| 036 (Bo- | | GCUAUGCUGUUUUG | | GGCUAGUCCGUUAUCAACUUGAAAAA | |
| 5) | | | | GUGGCACCGAGUCGGUGCUUUU | |
| ART- | 83 | GGGCGGCAAUGGUG | 168 | GGGCGGCAAUGGUGUAGCGGGUUUUA | 253 |
| 001-g- | | UAGCGGGUUUUAGA | | GAGCUAGAAAUAGCAAGUUAAAAUAA | |
| 037 (Bo- | | GCUAUGCUGUUUUG | | GGCUAGUCCGUUAUCAACUUGAAAAA | |
| 11) | | | | GUGGCACCGAGUCGGUGCUUUU | |
| ART- | 84 | CAUUGAUGGCAGGA | 169 | CAUUGAUGGCAGGACUGCCUGUUUUA | 254 |
| 001-g- | | CUGCCUGUUUUAGA | | GAGCUAGAAAUAGCAAGUUAAAAUAA | |
| 038 (Bo- | | GCUAUGCUGUUUUG | | GGCUAGUCCGUUAUCAACUUGAAAAA | |
| 51) | | | | GUGGCACCGAGUCGGUGCUUUU | |
| ART- | 85 | AAAGGCUGCUGAUG | 170 | AAAGGCUGCUGAUGACACCUGUUUUA | 255 |
| 001-g- | | ACACCUGUUUUAGA | | GAGCUAGAAAUAGCAAGUUAAAAUAA | |
| int | | GCUAUGCUGUUUUG | | GGCUAGUCCGUUAUCAACUUGAAAAA | |
| (NTLA- | | | | GUGGCACCGAGUCGGUGCUUUU | |
| 2001) | | | | | |
| ART- | 275 | ACACAAAUACCAGU | 308 | ACACAAAUACCAGUCCAGCAGUUUUA | 341 |
| 001-g- | | CCAGCAGUUUUAGA | | GAGCUAGAAAUAGCAAGUUAAAAUAA | |
| 064 | | GCUAUGCUGUUUUG | | GGCUAGUCCGUUAUCAACUUGAAAAA | |
| | | | | GUGGCACCGAGUCGGUGCUUUU | |
| ART- | 276 | UCUAGAACUUUGAC | 309 | UCUAGAACUUUGACCAUCAGGUUUUA | 342 |
| 001-g- | | CAUCAGGUUUUAGA | | GAGCUAGAAAUAGCAAGUUAAAAUAA | |
| 065 | | GCUAUGCUGUUUUG | | GGCUAGUCCGUUAUCAACUUGAAAAA | |
| | | | | GUGGCACCGAGUCGGUGCUUUU | |
| ART- | 277 | UUACAGCCACGUCU | 310 | UUACAGCCACGUCUACAGCAGUUUUA | 343 |
| 001-g- | | ACAGCAGUUUUAGA | | GAGCUAGAAAUAGCAAGUUAAAAUAA | |
| 066 | | GCUAUGCUGUUUUG | | GGCUAGUCCGUUAUCAACUUGAAAAA | |
| | | | | GUGGCACCGAGUCGGUGCUUUU | |
| ART- | 278 | ACACAAAUACCAGU | 311 | ACACAAAUACCAGUCCAGCGGUUUUA | 344 |
| 001-g- | | CCAGCGGUUUUAGA | | GAGCUAGAAAUAGCAAGUUAAAAUAA | |
| 067 | | GCUAUGCUGUUUUG | | GGCUAGUCCGUUAUCAACUUGAAAAA | |
| | | | | GUGGCACCGAGUCGGUGCUUUU | |
| ART- | 279 | AAAGGCTGCUGAUG | 312 | AAAGGCTGCUGAUGACACCUGUUUUA | 345 |
| 001-g- | | ACACCUGUUUUAGA | | GAGCUAGAAAUAGCAAGUUAAAAUAA | |
| 068 | | GCUAUGCUGUUUUG | | GGCUAGUCCGUUAUCAACUUGAAAAA | |
| | | | | GUGGCACCGAGUCGGUGCUUUU | |
| ART- | 280 | AAAGGCUGCTGAUG | 313 | AAAGGCUGCTGAUGACACCUGUUUUA | 346 |
| 001-g- | | ACACCUGUUUUAGA | | GAGCUAGAAAUAGCAAGUUAAAAUAA | |
| 069 | | GCUAUGCUGUUUUG | | GGCUAGUCCGUUAUCAACUUGAAAAA | |
| | | | | GUGGCACCGAGUCGGUGCUUUU | |
| ART- | 281 | AAAGGCUGCUGATG | 314 | AAAGGCUGCUGATGACACCUGUUUUA | 347 |
| 001-g- | | ACACCUGUUUUAGA | | GAGCUAGAAAUAGCAAGUUAAAAUAA | |
| 070 | | GCUAUGCUGUUUUG | | GGCUAGUCCGUUAUCAACUUGAAAAA | |
| | | | | GUGGCACCGAGUCGGUGCUUUU | |
| ART- | 282 | AAAGGCUGCUGAUG | 315 | AAAGGCUGCUGAUGACACCTGUUUUA | 348 |
| 001-g- | | ACACCTGUUUUAGA | | GAGCUAGAAAUAGCAAGUUAAAAUAA | |
| 071 | | GCUAUGCUGUUUUG | | GGCUAGUCCGUUAUCAACUUGAAAAA | |
| | | | | GUGGCACCGAGUCGGUGCUUUU | |
| ART- | 283 | AAAGGCUGCUGATG | 316 | AAAGGCUGCUGATGACACCTGUUUUA | 349 |
| 001-g- | | ACACCTGUUUUAGA | | GAGCUAGAAAUAGCAAGUUAAAAUAA | |
| 072 | | GCUAUGCUGUUUUG | | GGCUAGUCCGUUAUCAACUUGAAAAA | |
| | | | | GUGGCACCGAGUCGGUGCUUUU | |
| ART- | 284 | AAAGGCTGCTGATG | 317 | AAAGGCTGCTGATGACACCTGUUUUA | 350 |
| 001-g- | | ACACCTGUUUUAGA | | GAGCUAGAAAUAGCAAGUUAAAAUAA | |
| 073 | | GCUAUGCUGUUUUG | | GGCUAGUCCGUUAUCAACUUGAAAAA | |
| | | | | GUGGCACCGAGUCGGUGCUUUU | |
| ART- | 285 | AAGGCUGCUGAUGA | 318 | AAGGCUGCUGAUGACACCU | 351 |
| 001-g- | | CACCUGUUUUAGAG | | GUUUUAGAGCUAGAAAUAGCAAGUUA | |
| 074 | | CUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 286 | AGGCUGCUGAUGAC | 319 | AGGCUGCUGAUGACACCU | 352 |
| 001-g- | | ACCUGUUUUAGAGC | | GUUUUAGAGCUAGAAAUAGCAAGUUA | |
| 075 | | UAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 287 | GGCUGCUGAUGACA | 320 | GGCUGCUGAUGACACCU | 353 |
| 001-g- | | CCUGUUUUAGAGCU | | GUUUUAGAGCUAGAAAUAGCAAGUUA | |
| 076 | | AUGCUGUUUUG | | AAAUAAGGCUAGUCCGUUAUCAACUU | |
| | | | | GAAAAAGUGGCACCGAGUCGGUGCUU | |
| | | | | UU | |
| ART- | 288 | GCUGCUGAUGACAC | 321 | GCUGCUGAUGACACCU | 354 |
| 001-g- | | CUGUUUUAGAGCUA | | GUUUUAGAGCUAGAAAUAGCAAGUUA | |
| 077 | | UGCUGUUUUG | | AAAUAAGGCUAGUCCGUUAUCAACUU | |
| | | | | GAAAAAGUGGCACCGAGUCGGUGCUU | |
| | | | | UU | |
| ART- | 289 | AAGGCTGCTGATGA | 322 | AAGGCTGCTGATGACACCT | 355 |
| 001-g- | | CACCTGUUUUAGAG | | GUUUUAGAGCUAGAAAUAGCAAGUUA | |
| 078 | | CUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 290 | AGGCTGCTGATGAC | 323 | AGGCTGCTGATGACACCT | 356 |
| 001-g- | | ACCTGUUUUAGAGC | | GUUUUAGAGCUAGAAAUAGCAAGUUA | |
| 079 | | UAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 291 | GGCTGCTGATGACA | 324 | GGCTGCTGATGACACCT | 357 |
| 001-g- | | CCTGUUUUAGAGCU | | GUUUUAGAGCUAGAAAUAGCAAGUUA | |
| 080 | | AUGCUGUUUUG | | AAAUAAGGCUAGUCCGUUAUCAACUU | |
| | | | | GAAAAAGUGGCACCGAGUCGGUGCUU | |
| | | | | UU | |
| ART- | 292 | GCTGCTGATGACAC | 325 | GCTGCTGATGACACCT | 358 |
| 001-g- | | CTGUUUUAGAGCUA | | GUUUUAGAGCUAGAAAUAGCAAGUUA | |
| 081 | | UGCUGUUUUG | | AAAUAAGGCUAGUCCGUUAUCAACUU | |
| | | | | GAAAAAGUGGCACCGAGUCGGUGCUU | |
| | | | | UU | |
| ART- | 293 | TGUGUCUGAGGCUG | 326 | TGUGUCUGAGGCUGGCCCUA | 359 |
| 001-g- | | GCCCUAGUUUUAGA | | GUUUUAGAGCUAGAAAUAGCAAGUUA | |
| 082 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 294 | UGTGUCUGAGGCUG | 327 | UGTGUCUGAGGCUGGCCCUA | 360 |
| 001-g- | | GCCCUAGUUUUAGA | | GUUUUAGAGCUAGAAAUAGCAAGUUA | |
| 083 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 295 | UGUGTCUGAGGCUG | 328 | UGUGTCUGAGGCUGGCCCUA | 361 |
| 001-g- | | GCCCUAGUUUUAGA | | GUUUUAGAGCUAGAAAUAGCAAGUUA | |
| 084 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 296 | UGUGUCTGAGGCUG | 329 | UGUGUCTGAGGCUGGCCCUA | 362 |
| 001-g- | | GCCCUAGUUUUAGA | | GUUUUAGAGCUAGAAAUAGCAAGUUA | |
| 085 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 297 | UGUGUCUGAGGCTG | 330 | UGUGUCUGAGGCTGGCCCUA | 363 |
| 001-g- | | GCCCUAGUUUUAGA | | GUUUUAGAGCUAGAAAUAGCAAGUUA | |
| 086 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 298 | UGUGUCUGAGGCUG | 331 | UGUGUCUGAGGCUGGCCCTA | 364 |
| 001-g- | | GCCCTAGUUUUAGA | | GUUUUAGAGCUAGAAAUAGCAAGUUA | |
| 087 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 299 | TGTGTCTGAGGCUG | 332 | TGTGTCTGAGGCUGGCCCUA | 365 |
| 001-g- | | GCCCUAGUUUUAGA | | GUUUUAGAGCUAGAAAUAGCAAGUUA | |
| 088 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 300 | TGTGTCTGAGGCTG | 333 | TGTGTCTGAGGCTGGCCCUA | 366 |
| 001-g- | | GCCCUAGUUUUAGA | | GUUUUAGAGCUAGAAAUAGCAAGUUA | |
| 089 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 301 | TGTGTCTGAGGCTG | 334 | TGTGTCTGAGGCTGGCCCTA | 367 |
| 001-g- | | GCCCTAGUUUUAGA | | GUUUUAGAGCUAGAAAUAGCAAGUUA | |
| 090 | | GCUAUGCUGUUUUG | | AAAUAAGGCUAGUCCGUUAUCAACUU | |
| | | | | GAAAAAGUGGCACCGAGUCGGUGCUU | |
| | | | | UU | |
| ART- | 302 | ACACAAATACCAGU | 335 | ACACAAATACCAGUCCAGCAGUUUUA | 368 |
| 001-g- | | CCAGCAGUUUUAGA | | GAGCUAGAAAUAGCAAGUUAAAAUAA | |
| 091 | | GCUAUGCUGUUUUG | | GGCUAGUCCGUUAUCAACUUGAAAAA | |
| | | | | GUGGCACCGAGUCGGUGCUUUU | |
| ART- | 303 | ACACAAATACCAGT | 336 | ACACAAATACCAGTCCAGCAGUUUUA | 369 |
| 001-g- | | CCAGCAGUUUUAGA | | GAGCUAGAAAUAGCAAGUUAAAAUAA | |
| 092 | | GCUAUGCUGUUUUG | | GGCUAGUCCGUUAUCAACUUGAAAAA | |
| | | | | GUGGCACCGAGUCGGUGCUUUU | |
| ART- | 304 | UGUGUCUfGfAfGf | 337 | UGUGUCUfGfAfGfGCfUfGGCCCUA | 370 |
| 001-g- | | GCfUfGGCCCUA | | GUUUUAGAGCUAGAAAUAGCAAGUUA | |
| 093 | | GUUUUAGAGCUAUG | | AAAUAAGGCUAGUCCGUUAUCAACUU | |
| | | CUGUUUUG | | GAAAAAGUGGCACCGAGUCGGUGCUU | |
| | | | | UU | |
| ART- | 305 | CUUGGAUfUfCfAf | 338 | CUUGGAUfUfCfAfCCGfGfUGCCCU | 371 |
| 001-g- | | CCGfGfUGCCCU | | GUUUUAGAGCUAGAAAUAGCAAGUUA | |
| 094 | | GUUUUAGAGCUAUG | | AAAUAAGGCUAGUCCGUUAUCAACUU | |
| | | CUGUUUUG | | GAAAAAGUGGCACCGAGUCGGUGCUU | |
| | | | | UU | |
| ART- | 306 | ACUUGGAfUfUfCf | 339 | ACUUGGAfUfUfCfACCfGfGUGCCC | 372 |
| 001-g- | | ACCfGfGUGCCC | | GUUUUAGAGCUAGAAAUAGCAAGUUA | |
| 095 | | GUUUUAGAGCUAUG | | AAAUAAGGCUAGUCCGUUAUCAACUU | |
| | | CUGUUUUG | | GAAAAAGUGGCACCGAGUCGGUGCUU | |
| | | | | UU | |
| ART- | 307 | AAAGGCUfGfCfUf | 340 | AAAGGCUfGfCfUfGAfUfGACACCU | 373 |
| 001-g- | | GAfUfGACACCU | | GUUUUAGAGCUAGAAAUAGCAAGUUA | |
| 096 | | GUUUUAGAGCUAUG | | AAAUAAGGCUAGUCCGUUAUCAACUU | |
| | | CUGUUUUG | | GAAAAAGUGGCACCGAGUCGGUGCUU | |
| | | | | UU | |

The 118 sgRNA sequences shown in Table 4 (SEQ ID NOs: 171-255 and 341-373) were tested further in in vitro and in vivo assays.

### Example 2. Select gRNAs Showing High Editing Efficacy in Cell Lines

Various sgRNAs from Table 4 (SEQ ID NOs: 171-255 and 341-373) were screened for gene editing efficacy in multiple cell types electroporated with a ribonucleoprotein (RNP) complex containing spCas9 protein and indicated synthetic sgRNAs of Table 4 or lipotransfected with spCas9 mRNA and indicated synthetic sgRNAs of Table 4, which target human TTR. Percent editing was determined for sgRNAs comprising each guide sequence across each cell type and the guide sequences were then rank ordered based on highest Edit %. The following materials and methods were used in this study.

spCas9 Protein and sgRNA Delivery In Vitro

GRNA modifications. 2′-O-methyl (2′-O-Me) modifications were present in the last 3 nucleotides at both the 5′ and 3′ end. Phosphorothioate (PS) bonds were between the last four nucleotides at both the 5′ and 3′ end, Additional modification or replacements such as deoxyribonucleotides or 2′F were specifically indicated in examples.

HEK293 cell line. The human embryonic kidney adenocarcinoma cell line was cultured in DMEM media supplemented with 10% fetal bovine serum. Cells were plated at a density of 800,000-2,000,000 cells/well in a 6-well plate or 8,000-20,000 cells/well in a 96-well plate 24 hours prior to electroporation. Cells were electroporated with Celetrix electroporator (Celetrix, CTX-1500A) per the manufacturer's protocol. Cells were electroporated with a RNP complex containing Cas9 Nuclease (5-50 μmol), sgRNA (10-500 μmol) and Celetrix buffer.

HepG2 cell line. The human hepatocellular carcinoma cell line HepG2 was cultured in DMEM media supplemented with 10% fetal bovine serum. Cells were plated at a density of 1,000,000-1,500,000 cells/well in a 6-well plate or 8,000-22,000 cells/well in a 96-well plate 24 hours prior to electroporation. Cells were electroporated with Celetrix electroporator (Celetrix, CTX-1500A) per the manufacturer's protocol. Cells were electroporated with a RNP complex containing Cas9 Nuclease (5-50 μmol), sgRNA (10-500 μmol) and Celetrix buffer.

Huh7 cell line. The human hepatocellular carcinoma cell line Huh7 was cultured in DMEM media supplemented with 10% fetal bovine serum. Cells were plated at a density of 500,000-1,500,000 cells/well in a 6-well plate or 5,000-15,000 cells/well in a 96-well plate 24 hours prior to electroporation. Cells were electroporated with Celetrix electroporator (Celetrix, CTX-1500A) per the manufacturer's protocol. Cells were electroporated with a RNP complex containing Cas9 Nuclease (5-50 μmol), sgRNA (10-500 μmol) and Celetrix buffer.

spCas9 mRNA and sgRNA Delivery In Vitro

HEK293 cell line. The human embryonic kidney adenocarcinoma cell line was cultured in DMEM media supplemented with 10% fetal bovine serum. Cells were plated at a density of 800,000-2,000,000 cells/well in a 6-well plate or 8,000-20,000 cells/well in a 96-well plate 24 hours prior to transfection. Cells were transfected with Lipofectamine 3000 (ThermoFisher, Cat. L3000001) per the manufacturer's protocol. Cells were transfected with a lipoplex containing 0.5-500 ng Cas9 mRNA, 1-1,000 ng sgRNA and Lipofectamine 3000.

HepG2 cell line. The human hepatocellular carcinoma cell line HepG2 was cultured in DMEM media supplemented with 10% fetal bovine serum. Cells were plated at a density of 1,000,000-1,500,000 cells/well in a 6-well plate or 8,000-22,000 cells/well in a 96-well plate 24 hours prior to transfection. Cells were transfected with Lipofectamine RNAiMAX (ThermoFisher, Cat. 13778075) per the manufacturer's protocol. Cells were transfected with a lipoplex containing 1-500 ng Cas9 mRNA, 2-1,000 ng sgRNA and Lipofectamine RNAiMAX.

Huh7 cell line. The human hepatocellular carcinoma cell line Huh7 was cultured in DMEM media supplemented with 10% fetal bovine serum. Cells were plated at a density of 500,000-1,500,000 cells/well in a 6-well plate or 5,000-15,000 cells/well in a 96-well plate 24 hours prior to transfection. Cells were transfected with Lipofectamine MessengerMAX (ThermoFisher, Cat. LMRNA003) per the manufacturer's protocol. Cells were transfected with a lipoplex containing 1-500 ng Cas9 mRNA, 2-1,000 ng sgRNA and Lipofectamine MessengerMAX.

Primary human and cynomolgus hepatocytes. Primary human hepatocytes (PHH) (thermofisher or BioIVT) and primary cynomolgus hepatocytes (PCH) (BioIVT) were cultured per the manufacturer's protocol. In brief, the cells were thawed and resuspended in hepatocyte thawing medium with supplements followed by centrifugation at 100 g for 10 minutes for human and 80 g for 4 minutes for cyno. The supernatant was discarded and the pelleted cells resuspended in hepatocyte plating medium plus supplement pack. Cells were counted and plated on Bio-coat collagen I coated 96-well plates (ThermoFisher, Cat. 877272) at a density of 60,000 cells/well. Plated cells were allowed to settle and adhere for 6 or 24 hours in a tissue culture incubator at 37° C. and 5% CO₂ atmosphere. After incubation cells were checked for monolayer formation and media was replaced with hepatocyte culture medium with serum-free supplement pack. The next day, cells were transfected with Lipofectamine RNAiMAX (ThermoFisher, Cat. 13778075) per the manufacturer's protocol. Cells were transfected with a lipoplex containing 1-500 ng Cas9 mRNA, 2-1,000 ng sgRNA and Lipofectamine RNAiMAX.

### Transthyretin (TTR) ELISA Analysis in Cultured Cells

Cell (HepG2 or Huh7) supernatant or lysate was collected and isolated, then the TTR expression levels were determined using a Human Prealbumin (Transthyretin) ELISA Kit (Abcam, Cat. ab231920), according to manufacturer's protocol. Briefly, samples were serial diluted with kit sample diluent to a final dilution of 5,000-fold when measuring human TTR. 100 μL of the prepared standard or diluted serum samples were added to the ELISA plate, incubated for 30 minutes at room temperature then washed 3 times with provided wash buffer. 100 μL of detection antibody was then added to each well and incubated for 20 minutes at room temperature followed by 3 washes. 100 μL of substrate is added then incubated for 10 minutes at room temperature before the addition of 100 ul stop solution. The absorbance of the contents was measured on the Spectramax M5 plate reader with analysis using SoftmaxPro version 7.0 software. Serum TTR levels were calculated from the standard curve using 4 parameter logistic fit and expressed as ng/ml of serum or percent knockdown relative control (vehicle treated) cells.

### Western Blot Analysis of Intracellular TTR

The human hepatocellular carcinoma cell line, HepG2, was transfected as previously described. Six-days post-transfection, the media was removed and the cells were lysed with 50 ul RIPA buffer plus freshly added protease inhibitor mixture consisting of complete protease inhibitor cocktail (Sigma, Cat. 11697498001), 1 mM DTT, and 250 U/ml Benzonase (EMD Millipore, Cat. 71206-3). Cells were kept on ice for 30 minutes at which time NaCl (1 M final concentration) was added. Cell lysates were thoroughly mixed and retained on ice for 30 minutes. The whole cell extracts (“WCE”) were transferred to a PCR plate and centrifuged to pellet debris. A BCA assay (UU-BIO, Cat. U10007A) was used to assess protein content of the lysates. The BCA assay procedure was completed per the manufacturer's protocol. Extracts were stored at minus 20° C. prior to use. Western blots were performed to assess intracellular TTR protein levels. Lysates were denatured at 95° C. for 10 min. Western blots were run using the eZwest system on 8-12% Bis-Tris gels (Genscript) per the manufacturer's protocol. Blots were rinsed with TBST and probed with rabbit a-TTR monoclonal antibody (Abcam, Cat. Ab75815) at 1:2000 in TBST. β-actin was used as a loading control (Abcam, Cat. ab8226) at 1:5000 in TBST and incubated simultaneously with the TTR primary antibody. Blots were incubated and rinsed in TBST and probed with secondary antibodies to Mouse and Rabbit (ThermoFisher, Cat. PI35518 and PISA535571) at 1:25,000 each in TBST for 30 min at room temperature. After incubation, blots were rinsed 3 times for 5 min each in TBST and 2 times with PBS. Blots were visualized and analyzed using a ePhoto system (Genscript).

### Genomic DNA Isolation

Transfected cells were harvested post-transfection at 72 hours. The genomic DNA was extracted from each well of a 6-well plate using PureLink™ Genomic DNA Mini Kit (ThermoFisher, Cat. K182001) or each 10 cm dish using QuickExtract DNA Extraction Solution (LGC Lucigen, Cat. QE09050) per the manufacturer's protocol. All DNA samples were subjected to subsequent Sanger sequencing analyses, as described herein.

### Sanger Sequencing Analysis

To quantitatively determine the efficiency of editing at the target location in the genome and quickly shortlist potential sgRNAs, Sanger sequencing was utilized to identify the editing efficiency introduced by gene editing.

Primers were designed around the target site within the gene of interest (e.g. TTR), and the genomic area of interest was amplified.

Sanger sequencing was performed on 3730xl DNA Analyzer (ThermoFisher, Cat. 3730XL) per manufacturer's protocol. The raw sequencing files (.ab1) were analyzed for determining editing efficiency in online analysis tools (e.g., TIDE: tide.nki.nl/, ICE: ice.synthego.com/).

### Next-Generation Sequencing (“NGS”) Analysis

To quantitatively determine the efficiency and pattern of editing at the target location in the genome, sequencing was utilized to identify the presence of substitutions, insertions and deletions introduced by gene editing.

NGS was used to assess the efficiency and pattern of editing at the specific genomic target site. This approach allowed for the detection of any insertions, deletions, or substitutions introduced by the gene editing process in a quantitative manner.

Primers were designed around the target site within the gene of interest (e.g. TTR), and the genomic area of interest was amplified.

According to the Illumina manufacturer's protocols, additional PCR was performed to add chemistry for sequencing. Subsequently, the amplicons were sequenced using an Illumina NovaSeq 6000 instrument. The resulting reads were subjected to alignment against a reference genome, which could be the human reference genome (hg38), cynomolgus reference genome (mf5), rat reference genome (rn6), or mouse reference genome (mm39). Low quality score reads were eliminated prior to alignment. The reads were mapped to the reference genome and only those overlapping with the target region of interest were selected. The wild type reads and those containing an insertion, substitution, or deletion were determined and counted.

The editing percentage, also referred to as the “editing efficiency,” “percent editing,” or “indel frequency,” is determined by dividing the total number of sequence reads with insertions, deletions, or substitutions by the total number of sequence reads, which includes wild type reads.

### LNP Delivery In Vivo

Compositions for delivery of the protein and nucleic acid components of CRISPR/Cas to a cell, such as a cell in a patient, are needed. Particularly, compositions with useful properties for in vitro and in vivo delivery that can stabilize and deliver RNA components are of interest.

Herein, we provide lipid nanoparticle-based compositions with useful properties, in particular for delivery of CRISPR/Cas gene editing components. The LNP compositions comprise: an RNA component; and a lipid component, wherein the lipid component comprises: (1) about 45-55 mol-% amine lipid; (2) about 9-11 mol-% neutral lipid; and (3) about 1-5 mol-% PEG lipid, wherein the remainder of the lipid component is helper lipid, and wherein the N/P ratio of the LNP composition is about 3 to about 8.

Unless otherwise noted, CD-1 female mice or TTR-humanized mice, ranging 6-8 weeks of age were used in each study. Animals were weighed and grouped according to body weight for preparing dosing solutions based on group average weight. LNPs were dosed via the tail vein in a volume of 0.2 ml per animal (approximately 10 ml per kilogram body weight). The animals were observed every day for adverse effects. Body weight was measured at every other day post dose. Blood were collected through eye socket or heart puncture at various time points. For studies involving in vivo editing, animal was sacrificed and liver tissue was collected for DNA extraction and analysis.

### Transthyretin (TTR) ELISA Analysis Used in Animal Studies

Blood was collected and the serum was isolated. The total mouse TTR and human TTR serum levels were measured using Mouse Prealbumin ELISA kit (Abcam, Cat. ab282297) and Human Prealbumin ELISA kit (Abcam, Cat. ab231920), respectively, each following manufacture's protocol.

### Example 3. Targeted Sequencing for Validating Potential Off-Target Sites

Potential off-target sites are validated using targeted sequencing of the identified potential off-target sites.

In one approach, HepG2 cells are transfected with Cas9 mRNA and sgRNA as described in Example 1. The HepG2 cells are then lysed and primers flanking the potential off-target site(s) are used to generate an amplicon for NGS analysis. Identification of indels at a certain level (dependent on the overall indel level for all sites) validate potential off-target site, whereas the lack of indels found at the potential off-target site may indicate a false positive in the HepG2 dsDNA insertion assay.

Genomic DNA isolation. Transfected cells were harvested post-transfection at 72 hours. The genomic DNA was extracted from either each well of a 6-well plate using PureLink™ Genomic DNA Mini Kit (ThermoFisher, Cat. K182001) or each 10 cm dish using QuickExtract DNA Extraction Solution (LGC Lucigen, Cat. QE09050) per manufacturer's protocol.

All DNA samples were subjected to subsequent Sanger sequencing analyses and NGS analysis, as described in Example 2.

### Example 4. Screening of sgRNA Sequences

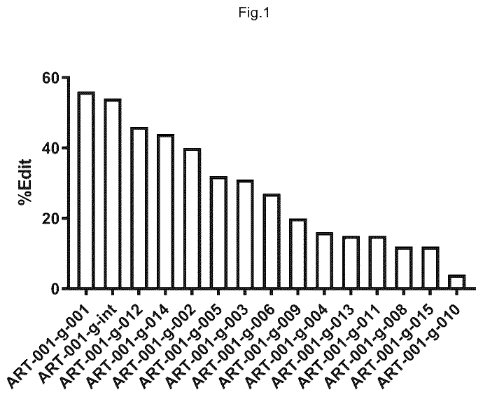

Screening of TTR guide RNAs in HEK293 sgRNAs targeting human TTR were delivered to HEK293 as described in Example 1. Percent editing was determined for sgRNAs comprising each guide sequence, and the guide sequences were then ordered based on highest % edit. The editing data are listed below in Table 5 and in FIG. 1.

TABLE 5
TTR editing data in HEK293 cells with Cas9 protein and sgRNAs

| Name | % Edit |
|---|---|
| ART-001-g-001 | 56 |
| ART-001-g-int | 54 |
| ART-001-g-012 | 46 |
| ART-001-g-014 | 44 |
| ART-001-g-002 | 40 |
| ART-001-g-005 | 32 |
| ART-001-g-003 | 31 |
| ART-001-g-006 | 27 |
| ART-001-g-009 | 20 |
| ART-001-g-004 | 16 |
| ART-001-g-013 | 15 |
| ART-001-g-011 | 15 |
| ART-001-g-008 | 12 |
| ART-001-g-015 | 12 |
| ART-001-g-010 | 4 |

### Screening of TTR Guide RNAs in HepG2

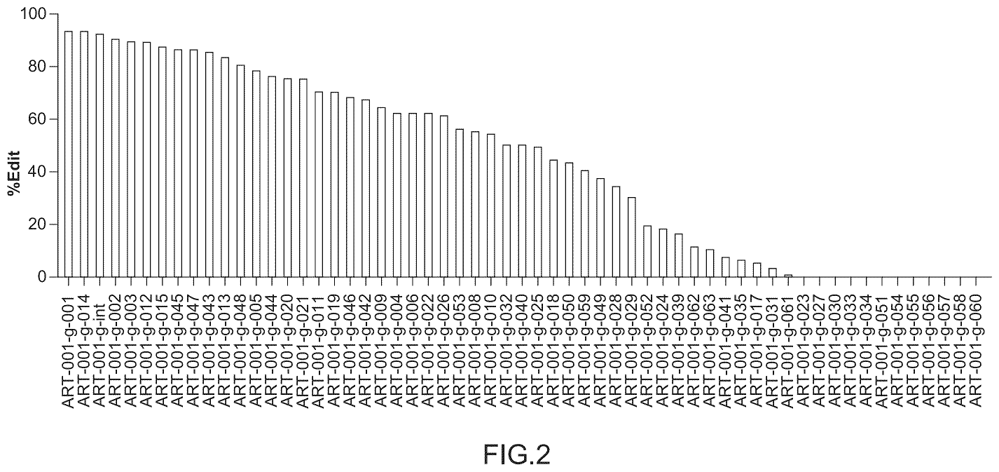

sgRNAs targeting human TTR were delivered to HepG2 as described in Example 1. Percent editing was determined for sgRNAs comprising each guide sequence and the guide sequences were then rank ordered based on highest % edit. The editing data are listed below in Table 6. The data are shown graphically in FIG. 2.

TABLE 6
TTR editing data in HepG2 cells with Cas9 protein and sgRNAs

| Name | % Edit |
|---|---|
| ART-001-g-001 | 94 |
| ART-001-g-014 | 94 |
| ART-001-g-int | 93 |
| ART-001-g-002 | 91 |
| ART-001-g-003 | 90 |
| ART-001-g-012 | 90 |
| ART-001-g-015 | 88 |
| ART-001-g-045 | 87 |
| ART-001-g-047 | 87 |
| ART-001-g-043 | 86 |
| ART-001-g-013 | 84 |
| ART-001-g-048 | 81 |
| ART-001-g-005 | 79 |
| ART-001-g-044 | 77 |
| ART-001-g-020 | 76 |
| ART-001-g-021 | 76 |
| ART-001-g-011 | 71 |
| ART-001-g-019 | 71 |
| ART-001-g-046 | 69 |
| ART-001-g-042 | 68 |
| ART-001-g-009 | 65 |
| ART-001-g-004 | 63 |
| ART-001-g-006 | 63 |
| ART-001-g-022 | 63 |
| ART-001-g-026 | 62 |
| ART-001-g-053 | 57 |
| ART-001-g-008 | 56 |
| ART-001-g-010 | 55 |
| ART-001-g-032 | 51 |
| ART-001-g-040 | 51 |
| ART-001-g-025 | 50 |
| ART-001-g-018 | 45 |
| ART-001-g-050 | 44 |
| ART-001-g-059 | 41 |
| ART-001-g-049 | 38 |
| ART-001-g-028 | 35 |
| ART-001-g-029 | 31 |
| ART-001-g-052 | 20 |
| ART-001-g-024 | 19 |
| ART-001-g-039 | 17 |
| ART-001-g-062 | 12 |
| ART-001-g-063 | 11 |
| ART-001-g-041 | 8 |
| ART-001-g-035 | 7 |
| ART-001-g-017 | 6 |
| ART-001-g-031 | 4 |
| ART-001-g-061 | 1 |
| ART-001-g-023 | 0 |
| ART-001-g-027 | 0 |
| ART-001-g-030 | 0 |
| ART-001-g-033 | 0 |
| ART-001-g-034 | 0 |
| ART-001-g-051 | 0 |
| ART-001-g-054 | 0 |
| ART-001-g-055 | 0 |
| ART-001-g-056 | 0 |
| ART-001-g-057 | 0 |
| ART-001-g-058 | 0 |
| ART-001-g-060 | 0 |

### Screening of TTR Guide RNAs in Primary Human Hepatocytes

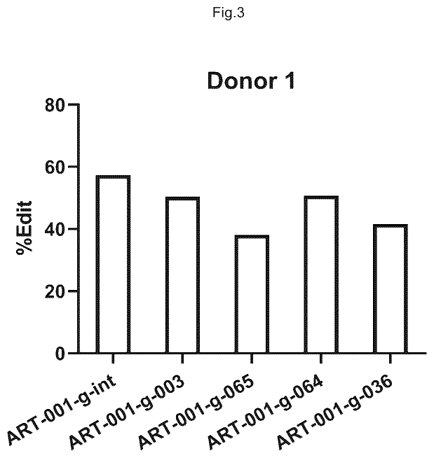

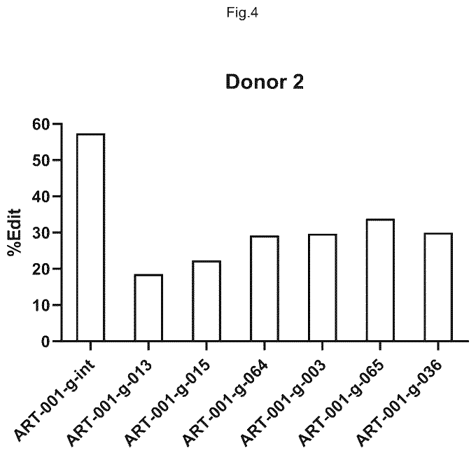

sgRNAs targeting human TTR were delivered to primary human hepatocytes from different donors using RNAi max as described in Example 1. Percent editing was determined for sgRNAs comprising each guide sequence, and the guide sequences were then rank ordered based on highest % edit. The editing data are listed below in Table 7 and 8. The data are shown graphically in FIGS. 3-4.

TABLE 7
TTR editing data in primary human hepatocytes
(Donor 1) with Cas9 mRNA and sgRNAs

| Name | % Edit |
|---|---|
| ART-001-g-int | 57 |
| ART-001-g-003 | 50 |
| ART-001-g-065 | 38 |
| ART-001-g-064 | 51 |
| ART-001-g-036 | 42 |

TABLE 8
TTR editing data in primary human hepatocytes
(Donor 2) with Cas9 mRNA and sgRNAs

| Name | % Edit |
|---|---|
| ART-001-g-int | 57 |
| ART-001-g-013 | 19 |
| ART-001-g-015 | 22 |
| ART-001-g-003 | 30 |
| ART-001-g-065 | 34 |
| ART-001-g-064 | 29 |
| ART-001-g-036 | 30 |

### Example 5. Dose Response of sgRNAs in HepG2 and PHH Cells

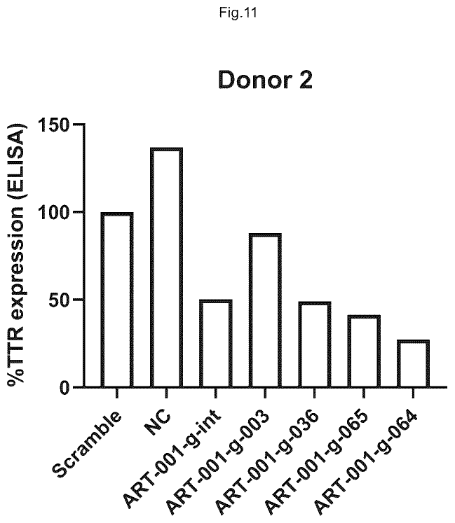

Lipid nanoparticle formulations of modified sgRNAs targeting human TTR sequences were tested on HepG2 or primary human hepatocytes in different concentration of RNA. HepG2 cells or primary human hepatocytes were plated as described in Example 1. Both cell lines were incubated at 37° C., 5% CO2 for 24 hours prior to treatment with LNPs. The LNPs used in the experiments detailed in Tables 20-22 were prepared using the cross-flow procedure described above but purified using PD-10 columns (GE Healthcare Life Sciences) and concentrated using Amicon centrifugal filter units (Millipore Sigma), each containing the specified sgRNA and Cas9 mRNA (SEQ ID NO: 1). The LNPs contained ALC-0315, Cholesterol, DSPC, and PEG2k-DMG in a 50:38:9:3 molar ratio, respectively, and had a N:P ratio of 6.0. LNPs were incubated in hepatocyte maintenance media containing 6% serum at 37° C., 5% CO2 for 5 minutes. Post incubation the LNPs were added onto the HepG2 cells or primary human hepatocytes in a 6-12 point 2-fold concentration increase. The cells were lysed 72 hours post treatment for Sanger Sequencing analysis as described in Example 1. Percent editing was determined for sgRNAs comprising each guide sequence and the guide sequences were then rank ordered based on EC50 values and maximum editing percent. The dose response curve data for the guide sequences in HepG2 cell lines or primary human hepatocytes is shown in FIGS. 11 through 13. The EC 50 values and maximum editing percent are listed below (Table 9 and 10).

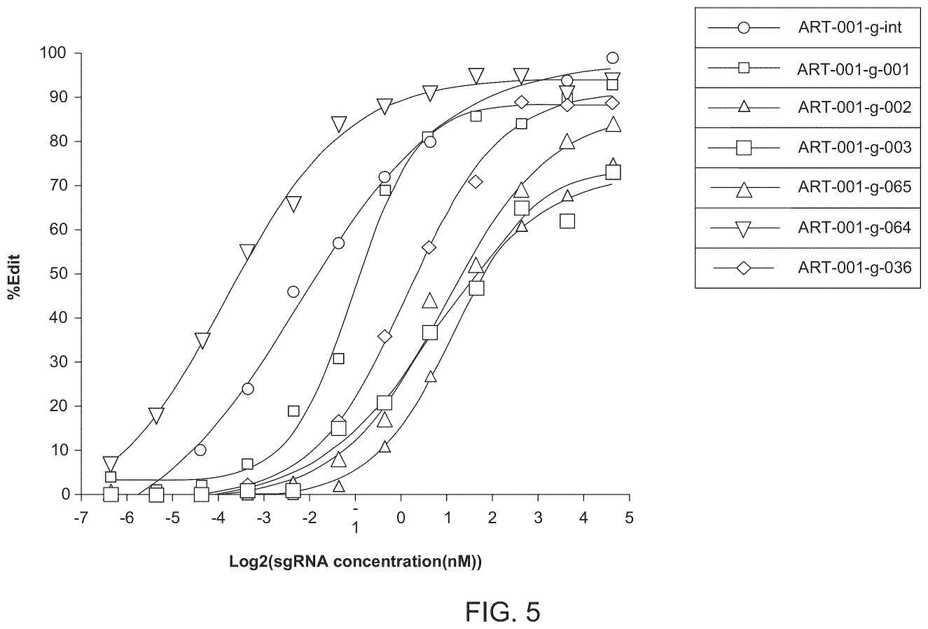

Table 9 shows the EC50 and maximum editing of the tested human specific TTR sgRNAs with Spy Cas9 protein on HepG2 as dose response curves. The data are shown graphically in FIG. 5.

TABLE 9
TTR editing data in HepG2 cells treated
with Cas9 protein and sgRNAs

| Name | EC50 | Max % Edit |
|---|---|---|
| ART-001-g-int | 0.006 | 98.22 |
| ART-001-g-001 | 0.092 | 88.86 |
| ART-001-g-002 | 14.220 | 74.23 |
| ART-001-g-003 | 4.665 | 73.79 |
| ART-001-g-065 | 8.972 | 86.65 |
| ART-001-g-064 | 0.0001 | 94.44 |
| ART-001-g-036 | 1.395 | 91.55 |

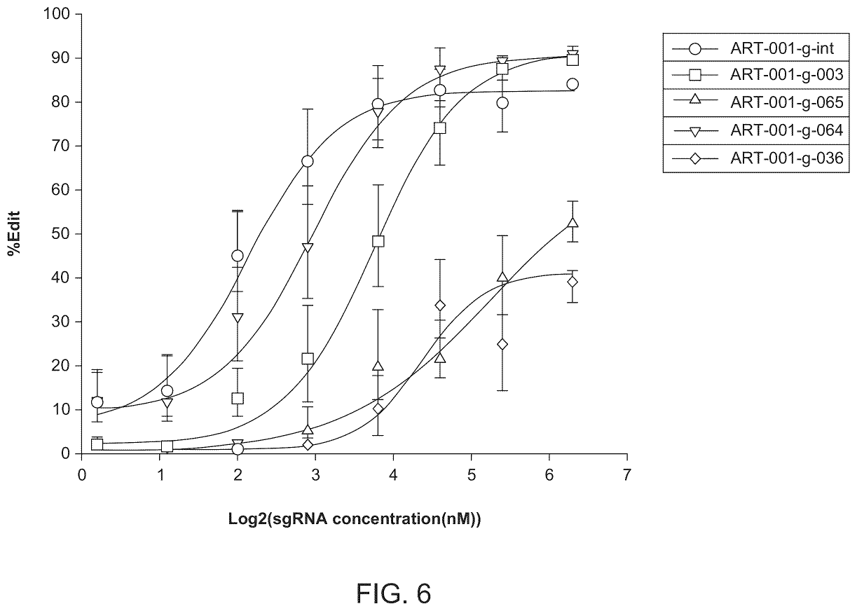

Table 10 shows the EC50 and maximum editing of the tested human specific TTR sgRNAs with Spy Cas9 mRNA formulated in LNP on HepG2 as dose response curves. The data are shown graphically in FIG. 6.

TABLE 10
TTR editing data in HepG2 cells treated with
Cas9 mRNA and sgRNAs formulated in LNP

| Name | EC50 | Max % Edit |
|---|---|---|
| ART-001-g-int | 4.2 | 82.92 |
| ART-001-g-003 | 13.4 | 91.67 |
| ART-001-g-065 | 44.1 | 68.73 |
| ART-001-g-064 | 7.5 | 90.77 |
| ART-001-g-036 | 14.5 | 41.37 |

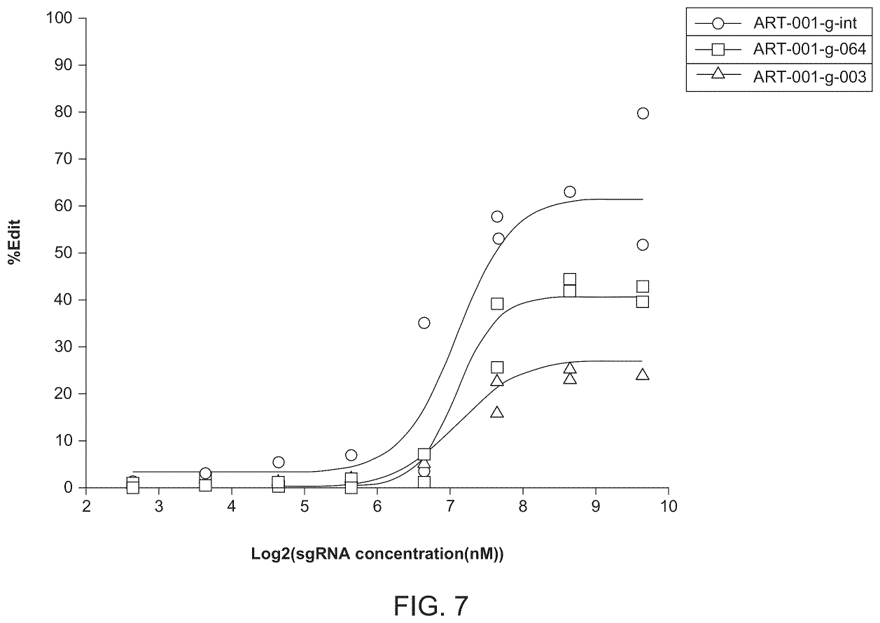

FIG. 7 shows the editing of the tested human specific TTR sgRNAs with Spy Cas9 mRNA formulated in LNP on primary human hepatocytes as concentration response curves.

### Example 6. TTR Protein Analysis

### Western Blot Analysis of Intracellular TTR

The primary human hepatocytes from different donors, were transfected as described in Example 1 with sgRNA comprising the guides from Table 3 and 4. The transfected pools of cells were cultured for further analysis. At 5 days post-transfection, cells were harvested and whole cell extracts (WCEs) were prepared and subjected to analysis by Western Blot as described in Example 1. WCEs were analyzed by Western Blot for reduction of TTR protein. Full length TTR protein has a predicted molecular weight of 16 kD. A band at this molecular weight was observed in the control lanes in the Western Blot.

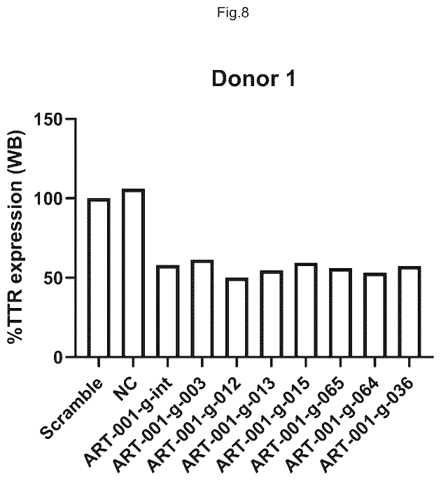

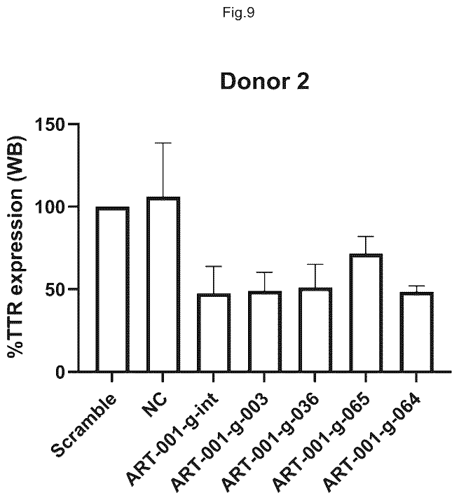

Percent reduction of TTR protein was calculated using the ePhoto software (Genscript). Beta-actin was used as a loading control and probed together with TTR. A ratio was calculated for the densitometry values for beta-actin within each sample compared to the total region encompassing the TTR band. Percent reduction of TTR protein was determined after the ratios were normalized to control lanes. Results are shown in FIGS. 8-9.

### ELISA Analysis of Secreted and Intracellular TTR

Primary human hepatocytes obtained from various donors were transfected with sgRNA containing the guides outlined in Tables 3 and 4, following the protocol outlined in Example 1. The transfected cell pools were cultured for additional analysis. After a period of 5 days following transfection, cell lysates or supernatants were collected and analyzed via ELISA using previously described methods.

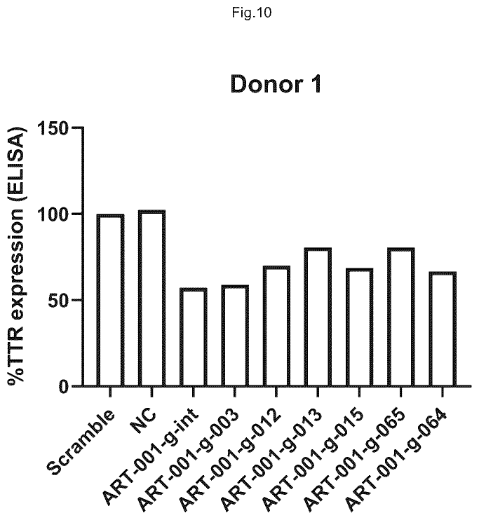

The percent reduction in beta-actin protein was calculated, and the percent reduction in TTR protein was determined after normalization to the levels observed in cells transfected with scramble sgRNA controls. Results are shown in FIGS. 10-11.

### Example 7. On Target Large Deletion or Insertion Analysis of sgRNA in PHH

About 2.8 million human primary hepatocytes were transfected by RNAiMax with 4000 ng of Cas9 mRNA and 800 ng of sgRNA. Three days later, the genomic DNA were harvested and around 10 kb of the sgRNA targeted sites were amplified by PCR by SuperFi II DNA Polymerase (ThermoFisher Sci). The sequencing was performed with a PacBio Sequel II system using an 8M SMRTCell. The percentage of large variants, which are defined as insertions or deletions larger than 10 base pairs, was determined by dividing the number of large variance variants by the total number of aligned reads.

TABLE 11
Large structure variances at the on-target
region in PHH cells for different sgRNAs

| | Large size variance |
|---|---|
| sgRNA | (>=10 bp) |
| ART-001-g-003 | 0.14% |
| ART-001-g-065 | 0.12% |
| ART-001-g-064 | 0.13% |
| ART-001-g-036 | 0.18% |

### Example 8. On Target Editing Efficiency of Truncated and/or Modification of sgRNA in HepG2

The 5′ end 20 guide sequences of the sgRNA were further modified by deoxyribonucleic acid replacement or deletion. 200,000˜ 400,000 HepG2 cells were electroporated with 5 μg of Cas9 protein and 2.5 μg of sgRNA. After 2 to 3 days, the genomic DNA was extracted, and the targeted regions were amplified with PCR and sent for Sanger sequencing. The editing efficiency were measured by ICE analysis.

TABLE 12
On target editing efficiency of truncated
and/or DNA-RNA hybrid sgRNA in HepG2 cells

| sgRNA name | Editing efficiency % |
|---|---|
| ART-001-g-int | 95 |
| ART-001-g-068 | 95 |
| ART-001-g-069 | 95 |
| ART-001-g-070 | 95 |
| ART-001-g-071 | 95 |
| ART-001-g-072 | 95 |
| ART-001-g-073 | 95 |
| ART-001-g-074 | 60 |
| ART-001-g-075 | 59 |
| ART-001-g-076 | 49 |
| ART-001-g-077 | 30 |
| ART-001-g-078 | 16 |
| ART-001-g-079 | 11 |
| ART-001-g-001 | 66 |
| ART-001-g-082 | 59 |
| ART-001-g-083 | 66 |
| ART-001-g-084 | 66 |
| ART-001-g-085 | 66 |
| ART-001-g-086 | 72 |
| ART-001-g-087 | 62 |
| ART-001-g-088 | 21 |
| ART-001-g-089 | 60 |

### Example 9. Double-Stranded DNA (dsDNA) Insertion Sites of TTR sgRNA in PHH Cells

An dsDNA insertion-based assay was used to screen for potential genomics off-target sites cleaved by Cas9 with the corresponding sgRNA. 1.4 million PHH cells were transfected by Lipofectamine RNAiMax with 20 pmol dsDNA, 2000 ng Cas9 mRNA and 400 ng sgRNA. After three days, the genomic DNA was extracted with OceanNano Tech PureBind Genomic DNA Isolation Kit and processed for NGS assay (See, e.g., Tsai et al., Nature Biotechnology 33, 187-197; 2015) in a NextSeq2000 sequencer. The dsDNA incorporation efficiency for each potential off-target site was calculated as the number of reads at this site divided by the reads at the on-target site (TTR). The top one and/or the sum of top 30 potential off-target sites' incorporation efficiency were used as a quantification readout for the off-target performance of sgRNAs.

TABLE 13
Potential off-target profiles in PHH cells by dsDNA insertion assay

| sgRNA | cell | total sites | Top 1/on | Top 30/on |
|---|---|---|---|---|
| ART-001-g-001 | Donor 1 | 132 | 2.389 | 7.632 |
| ART-001-g-001 | Donor 3 | 24 | 0.934 | 2.764 |
| ART-001-g-003 | Donor 1 | 33 | 0.040 | 0.350 |
| ART-001-g-003 | Donor 2 | 2 | 0.017 | 0.026 |
| ART-001-g-065 | Donor 2 | 0 | 0.000 | 0.000 |
| ART-001-g-065 | Donor 1 | 0 | 0.000 | 0.000 |
| ART-001-g-064 | Donor 2 | 1 | 0.005 | 0.005 |
| ART-001-g-064 | Donor 1 | 1 | 0.033 | 0.033 |
| ART-001-g-064 | Donor 3 | 3 | 0.016 | 0.036 |
| ART-001-g-036 | Donor 2 | 0 | 0.000 | 0.000 |
| ART-001-g-036 | Donor 1 | 1 | 0.034 | 0.034 |
| ART-001-g-int | Donor 1 | 103 | 0.327 | 1.531 |
| ART-001-g-int | Donor 3 | 12 | 0.138 | 0.556 |

### Example 10. Off-Target Analysis by dsDNA Insertion Assay for Differently Modified sgRNAs in HepG2

A dsDNA insertion-based assay was used to screen for potential genomics off-target sites cleaved by Cas9 with the corresponding sgRNA. HepG2 cells were maintained in MEM (Gibco) supplemented with 10% FBS (OPCEL) at 37° C. 5% CO2. 1 million HepG2 cells were electroporated in 4D-Nuclefector (LONZA, X-unit) with 200 pmol of dsDNA, 35 pmol of Cas9 (NEB, EnGen Spy Cas9 NLS) protein and 200 pmol of sgRNA (general biosystem or genewiz). The sgRNA contain either one or more of the modifications: deoxyribonucleotide replacement (DNA/RNA hybrid), truncation from the 5′ end, 2′O Fluoride modifications. Genomic DNA was extracted and processed for NGS assay (See, e.g., Tsai et al., Nature Biotechnology 33, 187-197; 2015) in a NextSeq6000 sequencer. The dsDNA incorporation efficiency for each potential off-target site was calculated as the number of reads at this site divided by the reads at the on-target site (TTR). The number of total sites found and top one off-target sites' incorporation efficiency were used as semi-quantification readouts for comparison between different sgRNA.

TABLE 14
Potential off-target sites in HepG2 cells for different sgRNAs
This table showed the effects between different sgRNAs.

| sgRNA | total sites | top1/on | top30/on |
|---|---|---|---|
| ART-001-g-001 | 154 | 0.770 | 1.580 |
| ART-001-g-002 | 28 | 0.121 | 0.367 |
| ART-001-g-003 | 14 | 0.019 | 0.043 |
| ART-001-g-065 | 4 | 0.000 | 0.001 |
| ART-001-g-064 | 7 | 0.199 | 0.206 |
| ART-001-g-036 | 13 | 0.004 | 0.016 |
| ART-001-g-int | 38 | 0.046 | 0.107 |

TABLE 15
Potential off-target sites in HepG2 cells for ART-001-g-001
containing fluorinated nucleotides or deoxy-ribonucleotides
This table showed the effects on off-target profile
of additional modifications in ART-001-g-001

| sgRNA | Additional modification | total sites | top1/on | Top30/on |
|---|---|---|---|---|
| ART-001-g-001 | | 154 | 0.770 | 1.580 |
| ART-001-g-093 | Fluoride at 2′ OH site of ART-001-g-001 | 154 | 1.092 | 3.515 |
| ART-001-g-082 | One T replacing U in ART-001-g-001 | 74 | 0.765 | 1.421 |
| ART-001-g-088 | four T replacing U in ART-001-g-001 | 40 | 0.386 | 0.629 |
| ART-001-g-090 | Six T replacing U in ART-001-g-001 | 18 | 0.025 | 0.087 |

TABLE 16
Potential off-target profile in HepG2 cells for ART-001-g-002
and ART-001-g003 containing fluorinated nucleotides.
This table showed the effects additional modifications in ART-001-g-002

| sgRNA | Additional modification | total sites | top1/on | Top30/on |
|---|---|---|---|---|
| ART-001-g-002 | | 28 | 0.121 | 0.367 |
| ART-001-g-094 | Fluoride at 2′ OH site in ART-001-g-002 | 68 | 0.065 | 0.155 |
| ART-001-g-003 | | 89 | 0.017 | 0.052 |
| ART-001-g-095 | Fluoride at 2′ OH site in ART-001-g-003 | 154 | 0.020 | 0.099 |

TABLE 17
Potential off-target sites in HepG2 cells for ART-001-g-int containing
fluorinated nucleotides or deoxyribonucleotides and/or truncations at 5′ end
This table showed the effects additional modifications in ART-001-g-int

| sgRNA | Additional modification | total sites | top1/on | Top30/on |
|---|---|---|---|---|
| ART-001-g-int | | 38 | 0.046 | 0.107 |
| ART-001-g-096 | Fluoride at 2′ OH site | 84 | 0.441 | 0.783 |
| ART-001-g-075 | Two nucleotides deletion at 5′ | 47 | 0.078 | 0.173 |
| ART-001-g-076 | three nucleotides deletion at 5′ | 51 | 0.047 | 0.094 |
| ART-001-g-079 | Two nucleotides deletion at 5′ | 42 | 0.015 | 0.034 |
| | and four T replacing U | | | |
| ART-001-g-080 | Three nucleotides deletion at 5′ | 80 | 0.053 | 0.093 |
| | and four T replacing U | | | |
| ART-001-g-073 | four T replacing U | 48 | 0.007 | 0.023 |

### Example 11. Off-Target Profiles in dsDNA Insertion Assay of DNA/RNA Hybrid sgRNA in PHH

In another experiment, 270,000 PHH cells were transfected with 1.5 ul Lipofectamine RNAiMax, 500 ng of Cas9 mRNA, 200 ng of sgRNA and 300 ng dsDNA. After three days, genomic DNA were extracted with FastPure Cell/Tissue DNA Isolation Mini Kit (Vazyme) and processed for NGS assay (See, e.g., Tsai et al., Nature Biotechnology 33, 187-197; 2015) in a NextSeq6000 sequencer. The dsDNA incorporation efficiency for each potential off-target site was calculated as the number of reads at this site divided by the reads at the on-target site (TTR). The top one and/or the sum of top 30 potential off-target sites' incorporation efficiency were used as a semi-quantification readout for comparison between different sgRNAs.

TABLE 18
potential off-target profile in PHH for sgRNA containing
deoxyribonucleotides

| | | Additional | | Total | Top 1 | Top 30 |
|---|---|---|---|---|---|---|
| Editor | gRNA | modification | Donor | sites | off/on | off/on |
| cas9 | ART- | | donor 1 | 132 | 2.389 | 7.632 |
| | 001-g-001 | | | | | |
| cas9 | ART- | 5 T replacing | donor 1 | 4 | 0.145 | 0.350 |
| | 001-g-089 | U in ART- | | | | |
| | | 001-g-001 | | | | |
| cas9 | ART- | 6 T replacing | donor 1 | 2 | 0.069 | 0.105 |
| | 001-g-090 | U in ART- | | | | |
| | | 001-g-001 | | | | |
| cas9 | ART- | | donor 2 | 24 | 0.934 | 2.764 |
| | 001-g-001 | | | | | |
| cas9 | ART- | 6 T replacing | donor 2 | 4 | 0.033 | 0.075 |
| | 001-g-090 | U in ART- | | | | |
| | | 001-g-001 | | | | |
| cas9Trex | ART- | 6 T replacing | donor 2 | 4 | 0.134 | 0.217 |
| | 001-g-090 | U in ART- | | | | |
| | | 001-g-001 | | | | |
| cas9Trex | ART- | | donor 2 | 10 | 0.215 | 0.630 |
| | 001-g-001 | | | | | |
| cas9 | ART- | | donor 1 | 1 | 0.033 | 0.033 |
| | 001-g-064 | | | | | |
| cas9 | ART- | 1 T replacing | donor 1 | 0 | 0.000 | 0.000 |
| | 001-g-091 | U in ART- | | | | |
| | | 001-g-064 | | | | |
| cas9 | ART- | 2 T replacing | donor 1 | 0 | 0.000 | 0.000 |
| | 001-g-092 | U in ART- | | | | |
| | | 001-g-064 | | | | |
| cas9 | ART- | | donor 2 | 3 | 0.016 | 0.036 |
| | 001-g-064 | | | | | |
| cas9 | ART- | 2 T replacing | donor 2 | 2 | 0.057 | 0.077 |
| | 001-g-092 | U in ART- | | | | |
| | | 001-g-064 | | | | |
| cas9Trex | ART- | | donor 2 | 2 | 0.007 | 0.015 |
| | 001-g-064 | | | | | |
| cas9Trex | ART- | 2 T replacing | donor 2 | 9 | 0.537 | 0.843 |
| | 001-g-092 | U in ART- | | | | |
| | | 001-g-064 | | | | |

### Example 12. Analysis of the Large Structural Variants of TTR sgRNA in HepG2 Cells

HepG2 cells were maintained in MEM (Gibco) supplemented with 10% FBS (OPCEL) at 37° C. 5% CO2. 4 million HepG2 cells were electroporated in 4D-Nuclefector (LONZA, X-unit) with 1 nmol of Cas9 protein and 140 pmol of sgRNA. Three days later, the large structural variants were analyzed by a NGS methods as in Yin et al. Cell Discovery 5:18; 2019. The translocations events were quantified as percentage of total genomic editing events.

TABLE 19
large structural variants around on-target genomic
region by Cas9 and different sgRNAs in HepG2 cells

| sgRNA | Translocation |
|---|---|
| ART-001-g-001 | 0.80% |
| ART-001-g-003 | 0.02% |
| ART-001-g-065 | 0.06% |
| ART-001-g-064 | 0.06% |
| ART-001-g-036 | 0.03% |
| ART-001-g-int | 0.06% |

### Example 13. Large Structure Variances Analysis of TTR sgRNA in the Presence or Absence of a Cas9 and a Fused Exonuclease

HepG2 cells were electroporated with Cas9 mRNA or Cas9-Trex mRNA (as in Yin, J., Lu, C. et al. Nat Commun 13, 1204; 2022) and sgRNA. Three days later, the genomic DNA were harvested. The large structure variances were analyzed by a NGS methods as in Yin et al. Cell Discovery 5:18; 2019. The translocations events were quantified as percentage of total genomic editing events.

TABLE 20
Large structural varaints around on-target genomic
region by cas9 with or without a fused exonuclease.

| sgRNA | Editor | Translocation |
|---|---|---|
| ART-001-g-001 | Cas9 | 1.72% |
| ART-001-g-001 | Cas9Trex | 0.28% |
| ART-001-g-003 | Cas9 | 0.01% |
| ART-001-g-003 | Cas9Trex | 0.01% |

### Example 14. Off-Target Analysis of sgRNA by Amplicon-Based NGS

350,000 PHH cells were transfected with 1.5 ul Lipofectamine RNAiMax, 500 ng Cas9 or Cas9-Trex mRNA (SEQ ID 355) and 100 ng sgRNA. After three days, the genomic DNA was extracted with Zymo Genomic DNA Clean & Concentrator-25. The editing at the on target and three top off target sites were amplified by PCR with Thermofisher Phusion U Green Multiplex PCR Master Mix. PCR product was purified with KAPA Pure and sequenced at an illumina NextSeq2000 platform or send for Sanger sequencing. To account for differences in transfection or editing efficiencies between samples, the off-target site editing efficiency was normalized by dividing it by the on-target efficiency within the same sample.

TABLE 21
Off-target divided by on target reads from for differently modified sgRNA
The table shows the editing efficiencies of the three off-target sites divided by the on-
target editing efficiency. An empirically determined detection limit 0.1% was applied.

| | | OT | | Off/ON |
|---|---|---|---|---|
| gRNA | additional modification | site | locations | efficiency % |
| ART-001-g-001 | | 2 | chr15: 66,598,162-66,598,181 | 18.51 |
| ART-001-g-001 | | 5 | chr21: 43,375,900-43,375,919 | 18.92 |
| ART-001-g-089 | 5 T replacing U in ART-001-g-001 | 2 | chr15: 66,598,162-66,598,181 | 0.11 |
| ART-001-g-089 | 5 T replacing U in ART-001-g-001 | 5 | chr21: 43,375,900-43,375,919 | 0.12 |
| ART-001-g-090 | 6 T replacing U in ART-001-g-001 | 2 | chr15: 66,598,162-66,598,181 | 0.16 |
| ART-001-g-090 | 6 T replacing U in ART-001-g-001 | 5 | chr21: 43,375,900-43,375,919 | 0.10 |
| ART-001-g-064 | | 1 | chr14: 77,332,158-77,332,177 | 2.35 |
| ART-001-g-064 | | 2 | chr5: 91,397,024-91,397,043 | 0.20 |
| ART-001-g-091 | 1 T replacing U in ART-001-g-064 | 1 | chr14: 77,332,158-77,332,177 | 1.35 |
| ART-001-g-091 | 1 T replacing U in ART-001-g-064 | 2 | chr5: 91,397,024-91,397,043 | 0.13 |
| ART-001-g-092 | 2 T replacing U in ART-001-g-064 | 1 | chr14: 77,332,158-77,332,177 | 1.43 |
| ART-001-g-092 | 2 T replacing U in ART-001-g-064 | 2 | chr5: 91,397,024-91,397,043 | 0.13 |
| ART-001-g-int | | 1 | chr2: 192,658,563-192,658,582 | 16.32 |
| ART-001-g-int | | 4 | chr20: 9,216,119-9,216,138 | 5.61 |
| ART-001-g-072 | 2 T replacing U in ART-001-g-int | 1 | chr2: 192,658,563-192,658,582 | 2.53 |
| ART-001-g-072 | 2 T replacing U in ART-001-g-int | 4 | chr20: 9,216,119-9,216,138 | 0.91 |
| ART-001-g-073 | 4 T replacing U in ART-001-g-int | 1 | chr2: 192,658,563-192,658,582 | 0.50 |
| ART-001-g-073 | 4 T replacing U in ART-001-g-int | 4 | chr20: 9,216,119-9,216,138 | 0.09 |

### Example 15. On and Off-Target Efficiency of DNA/RNA Hybrid sgRNA in PHH Cells with LNP Delivery

60,000 PHH cells were transfected with LNP containing Cas9 mRNA and sgRNA. The dose was between 0 to 1500 ng of total RNA per well. After three days, the genomic DNA were extracted with FastPure Cell/Tissue DNA Isolation Mini Kit (Vazyme). The on and some off target sites were amplified by PCR with Taq Pro Multiplex DNA Polymerase (Vazyme) and purified with VAHTS DNA Clean Beads (Vazyme N411-01). The NGS sequencing as done on an Illumina NovaSeq6000 platform.

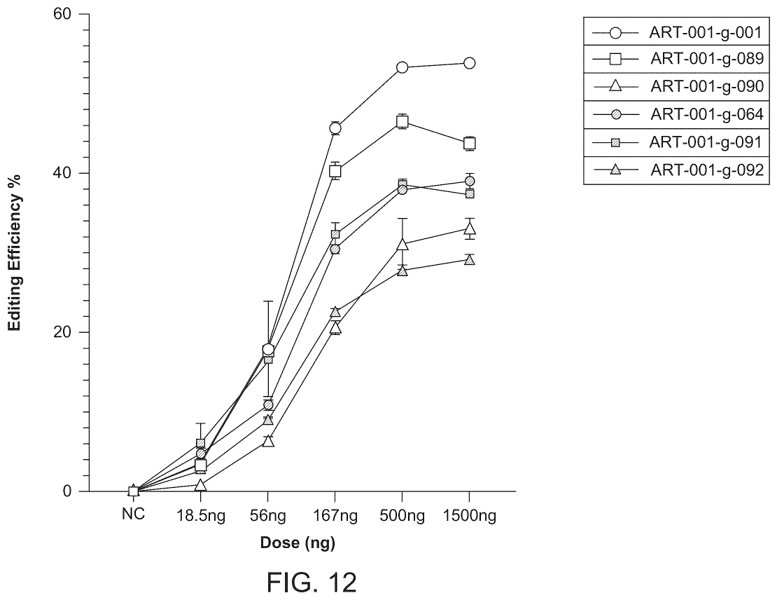

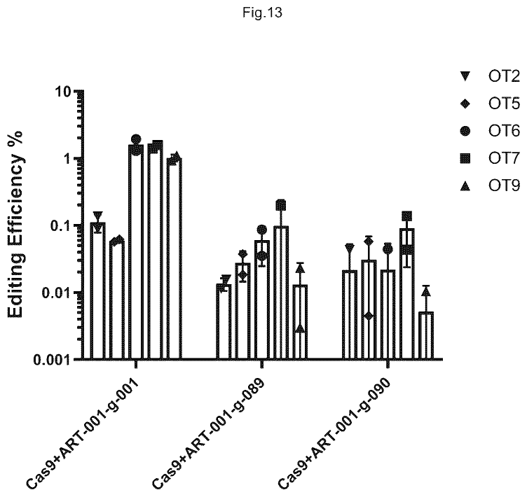

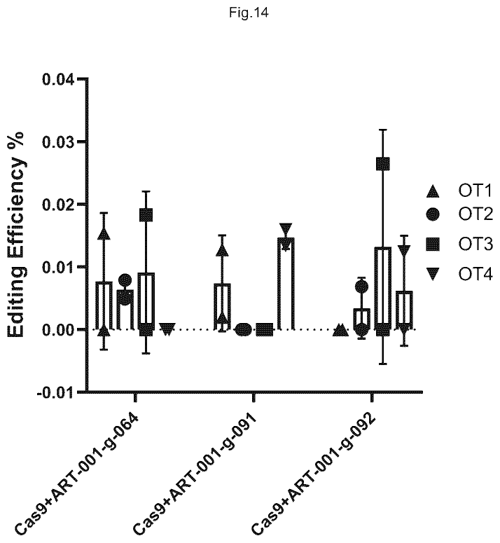

The dose response data were shown graphically in FIG. 12. The off-target amp-seq at the maximum dose data were shown in FIGS. 13-14.

### Example 16. On and Off-Target Analysis of Modification of sgRNA with Cas9-Trex with Lipofection

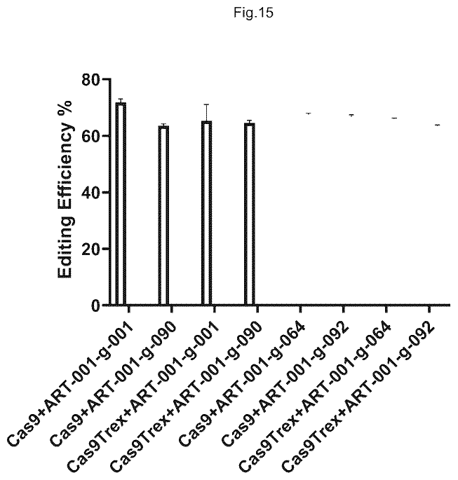

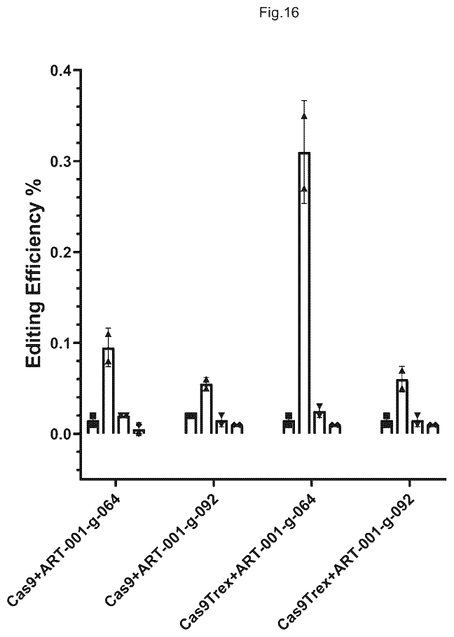

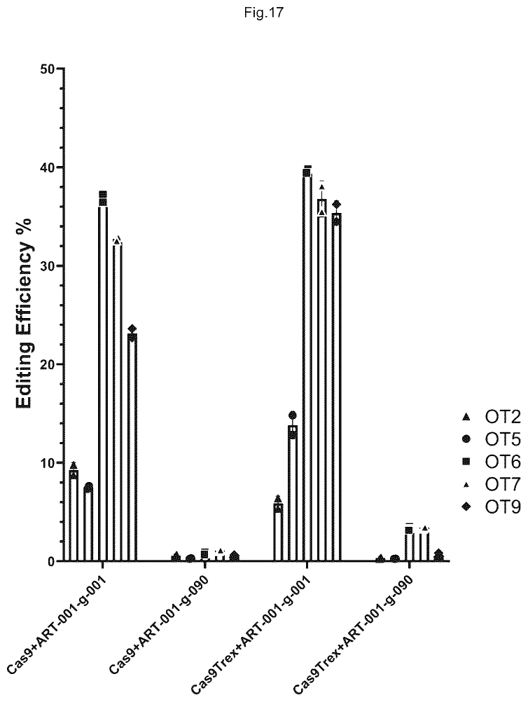

270,000 PHH cells were transfected with 1.5 ul Lipofectamine RNAiMax, 500 ng Cas9 or Cas9-Trex mRNA and 100 ng sgRNA in duplicates. After three days, the genomic DNA was extracted with FastPure Cell/Tissue DNA Isolation Mini Kit (Vazyme). The on and off-target sites were amplified by PCR with Taq Pro Multiplex DNA Polymerase (Vazyme) and purified with VAHTS DNA Clean Beads (Vazyme N411-01). The NGS sequencing was done on an Illumina NovaSeq6000 platform. FIG. 15 shows graphically the on-target genetic editing efficiency of the combination of exonuclease fusion and sgRNA modification. FIG. 16 shows graphically the editing efficiencies of the top 4 off target sites (Table 22) with or without exonuclease and/or sgRNA modification for some sgRNA. FIG. 17 shows graphically the editing efficiencies of the top 5 off target sites (Table 23) with or without exonuclease and/or sgRNA modification for some sgRNA.

TABLE 22
Off-target site locations

| Off-target sites | Chromosome locations |
|---|---|
| OT1 | chr14: 77,332,158-77,332,177 |
| OT2 | chr5: 91,397,024-91,397,043 |
| OT3 | chr6: 73,201,227-73,201,246 |
| OT4 | chr5: 26,758,031-26,758,050 |

TABLE 23
Off-target site locations

| Off-target sites | Chromosome locations |
|---|---|
| OT2 | chr15: 66,598,162-66,598,181 |
| OT5 | chr21: 43,375,900-43,375,919 |
| OT6 | chr3: 125,963,325-125,963,344 |
| OT7 | chr3: 123,998,013-123,998,032 |
| OT9 | chr4: 7,773,740-7,773,759 |

### Examples 17. LNP Deliver to Mice Carrying Wt (Murine) TTR

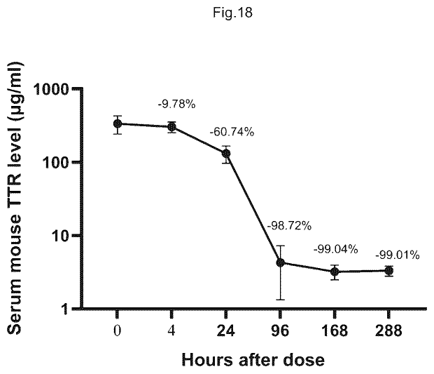

Mice (females from Beijing Vital River Laboratory Animal Technology Co., Ltd., approximately 6-10 weeks) were adminstered a LNP formulation, prepared using cross-flow and TFF procedures as described in example 1 containing a sgRNA (ART-001-g-066; SEQ ID NO: 277) and Cas9 mRNA in a 1:1 ratio by weight. The LNP had a N:P ratio of 3 and contained ALC0315, DSPC, Cholesterol, and PEG2k-DMG at a 50:10:38.5:1.5 molar ratio. Animals were treated with one single dose at 3 mg/kg (total RNA content). Blood was collected at 0, 4, 24, 96, 168 and 288 hours post dose. The group contained 5 mice. Serum TTR was determined using ELISA. Results for serum mouse TTR levels are shown in FIG. 18. Serum TTR levels were decreased by 99% at 168 hours post dose.

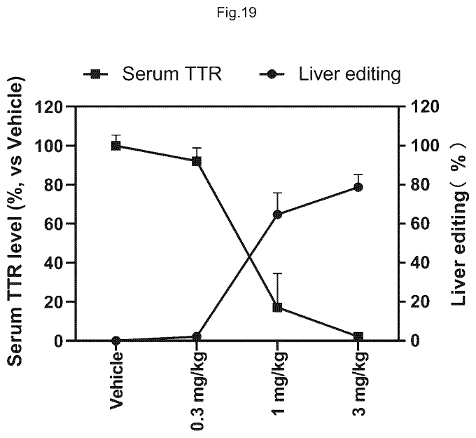

In another set of experiments using wild type CD-1 mice, a LNP formulation prepared using cross-flow and TFF procedures as described above containing sgRNA (ART-001-g-066; SEQ ID NO: 277 and Cas9 mRNA in a 1:1 ratio by weight. The LNP contained ALC0315, DSPC, Cholesterol, and PEG2k-DMG at a 50:10:38.5:1.5 molar ratio, respectively, and a N:P ratio of 3. Dose levels were at 3, 1, or 0.3 mg/kg (n=3/group). Liver editing results were measured using primers designed to amplify the region of interest for Sanger analysis. Results of mouse serum TTR and liver editing are shown in FIG. 19. A dose dependent effect of both serum TTR and liver editing was evident.

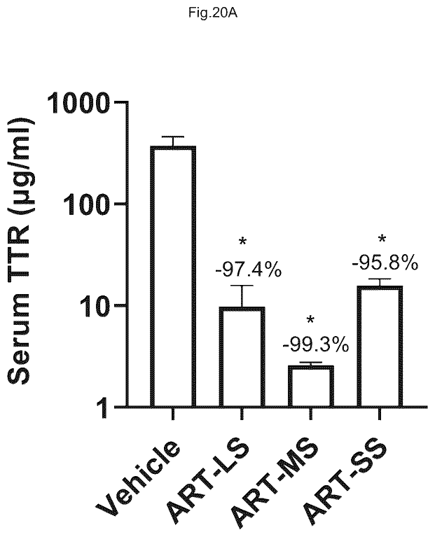

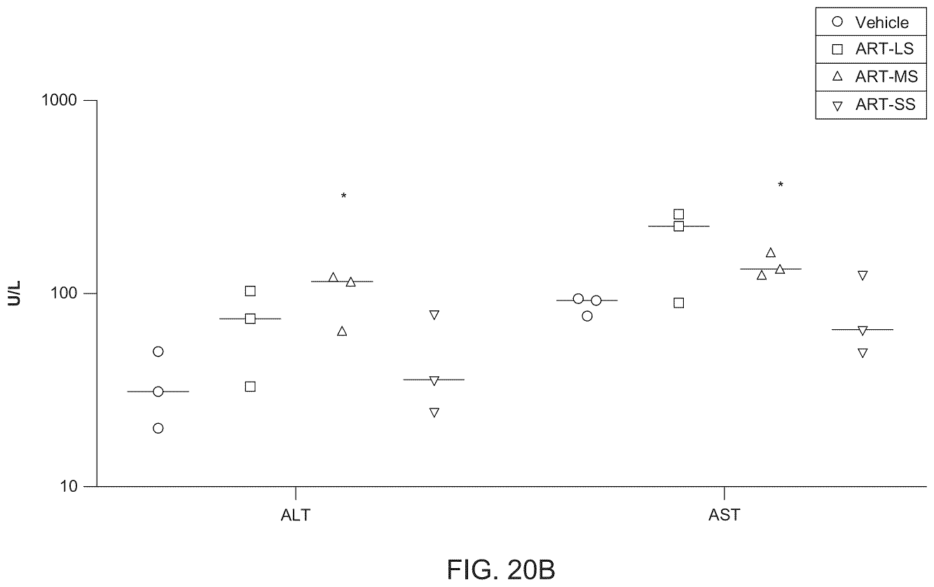

In the separate experiments, different sizes of LNPs as shown in Table 24 were prepared using the cross-flow procedure and concentrated using the centrifugal filter units. LNPs contained Cas9 mRNA in a 1:1 weight ratio to the sgRNA (ART-001-g-066; SEQ ID NO: 277. The LNPs contained ALC0315, DSPC, Cholesterol, and PEG2k-DMG at a 50:10:38.5:1.5 molar ratio. Animals were dosed at 1 mg/kg (total RNA content). Blood was collected at 7 days post treatment for serum TTR levels and blood chemistry analysis. More than 95% reduction of serum TTR levels was observed in all formulations, while the LNP with averaged size of 65 nm showed minimal risk of liver injury, as presented in FIGS. 20A-B.

TABLE 24
LNP formulations for in vivo studies

| | | | Z-Ave Size |
|---|---|---|---|
| Group | Guide | N:P | (nm) |
| ART-LS | ART-001-g-066 | 3 | 147 |
| ART-MS | ART-001-g-066 | 3 | 91 |
| ART-SS | ART-001-g-066 | 3 | 65 |

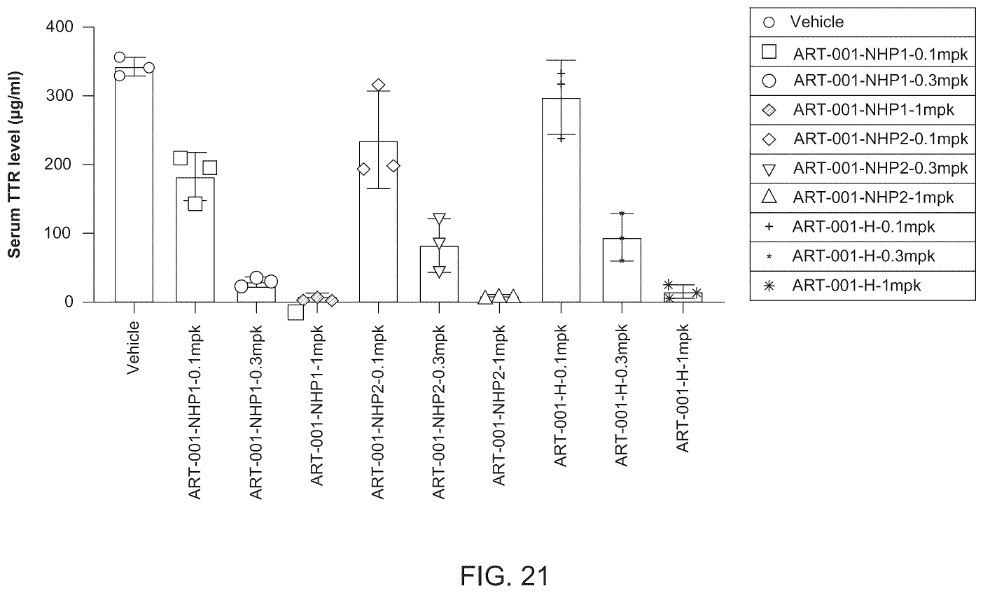

In another study, editing was assessed with different doses and lipids concentrations in compositions comprising ALC0315. The Cas9 mRNA was formulated as LNPs with a guide RNA targeting TTR (ART-001-g-067; SEQ ID NO: 278). These LNPs were formulated at a 1:1 or 1:2 weight ratio of single sgRNA and Cas9 mRNA. The LNPs were assembled using the cross-flow procedure with compositions as described in Table 25. LNP compositions were analyzed for average particle size, polydispersity (PDI), total RNA content and encapsulation efficiency of RNA as described. Analysis of average particle size, PDI, total RNA content and encapsulation efficiency of RNA are shown in Table 25. CD-1 female mice were dosed i.v. at 0.1, 0.3, and 1 mg/kg. Blood was collected for serum TTR analysis at 7 days post-treatment. Serum TTR results are shown in FIG. 21.

TABLE 25
Composition analytics of LNP formulations

| | | EE | Z-Ave Size | | mRNA:sgRNA |
|---|---|---|---|---|---|
| LNP | Composition | (%) | (nm) | PDI | (weight) |
| ART-001-NHP1 | 50:10:38.5:1.5 | 92 | 91 | 0.07 | 1:1 |
| ART-001-NHP2 | 50:9.5:37.5:3 | 74 | 69 | 0.08 | 2:1 |
| ART-001-H | 50:9.5:37.5:3 | 88 | 64 | 0.06 | 2:1 |

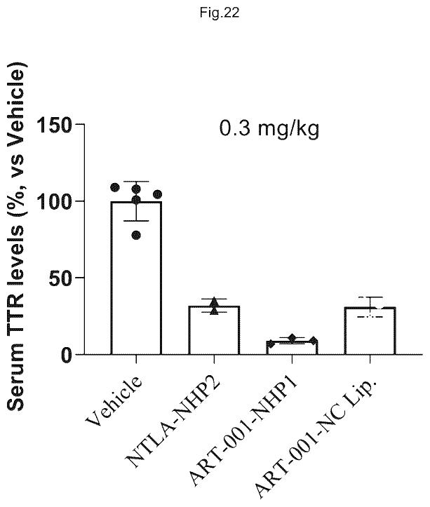

To assess the editing efficiency of LNP comprising different amine lipids, LNPs were formulated with a guide RNA targeting TTR (ART-001-g-067; SEQ ID NO: 278) and Cas9 mRNA. The LNPs were assembled using the cross-flow procedure with compositions as described in Table 26. LNP compositions were analyzed for average particle size, PDI and encapsulation efficiency of RNA. CD-1 female mice were dosed i.v. at 0.3 mg/kg. Blood was collected for serum TTR levels analysis at 7 days post-treatment. Serum TTR results are shown in FIG. 22.

TABLE 26
Composition analytics of LNP formulations

| | | | EE | Z-Ave Size | |
|---|---|---|---|---|---|
| LNP | Amine lipid | Composition | (%) | (nm) | PDI |
| NTLA-NHP2 | LP01 | 50:9:38:3 | 85 | 68 | 0.04 |
| ART-001-NHP1 | ALC0513 | 50:10:38.5:1.5 | 92 | 91 | 0.07 |
| ART-001-NC Lip. | SGM10 | 50:9.5:37.5:3 | 96 | 64 | 0.05 |

### Examples 18. LNP Delivery to Humanized TTR Mice

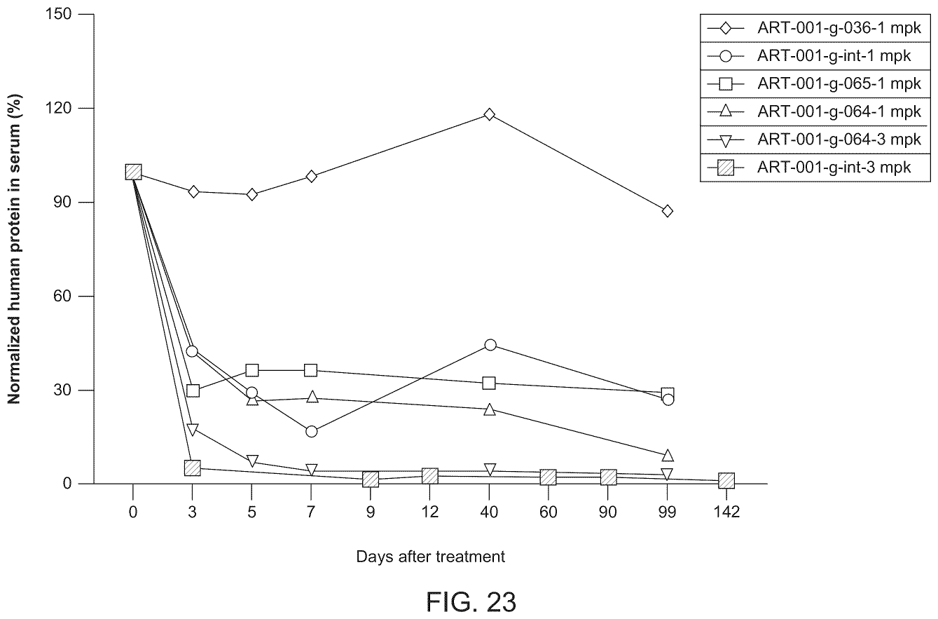

The mice were engineered by H11-locus knock-in coding region of mutant human TTR (V30M) gene. These humanized mice were dosed with LNP formulations containing the guide RNAs targeting human TTR. LNPs contained Cas9 mRNA in a 2:1 ratio by weight to respective guide RNA. The LNPs contained ALC0315, distearoylphosphatidlycholine (DSPC), Cholesterol, and PEG2k-DMG at a 50:10:38.5:1.5 molar ratio. Animals were dosed at 1 or 3 mg/kg (total RNA content). Blood was collected at various time points for serum TTR analysis. Serum TTR results are shown in FIG. 23.

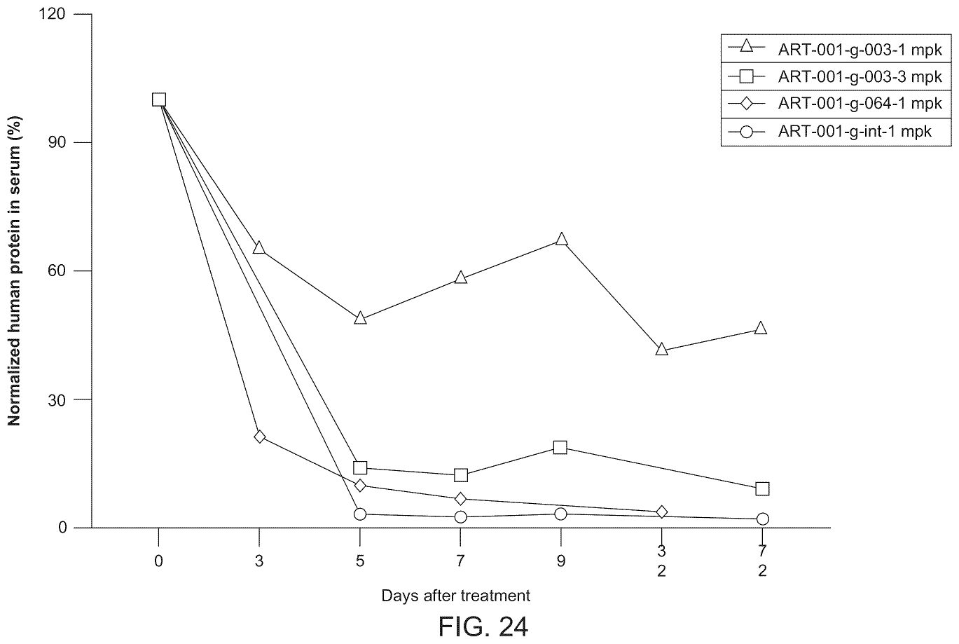

In another set of experiments, humanized mice were engineered such that a region of the endogenous murine ttr locus was deleted and replaced with an orthologous mutant human TTR (V30M) sequence so that the locus encodes a human TTR (V30M) protein. The LNP formulations were prepared using cross-flow and TFF procedures as described above containing guide RNAs targeting human TTR and Cas9 mRNA in a 1:2 ratio by weight. The LNP contained ALC0315, DSPC, Cholesterol, and PEG2k-DMG at a 50:10:38.5:1.5 molar ratio, respectively, and N:P ratio of 3. Dosing levels were at 1, or 3 mg/kg (n=1/group). Results for serum human TTR levels are shown in FIG. 24.

### Example 19. In Vivo Editing Efficiency of DNA/RNA Hybrid sgRNA

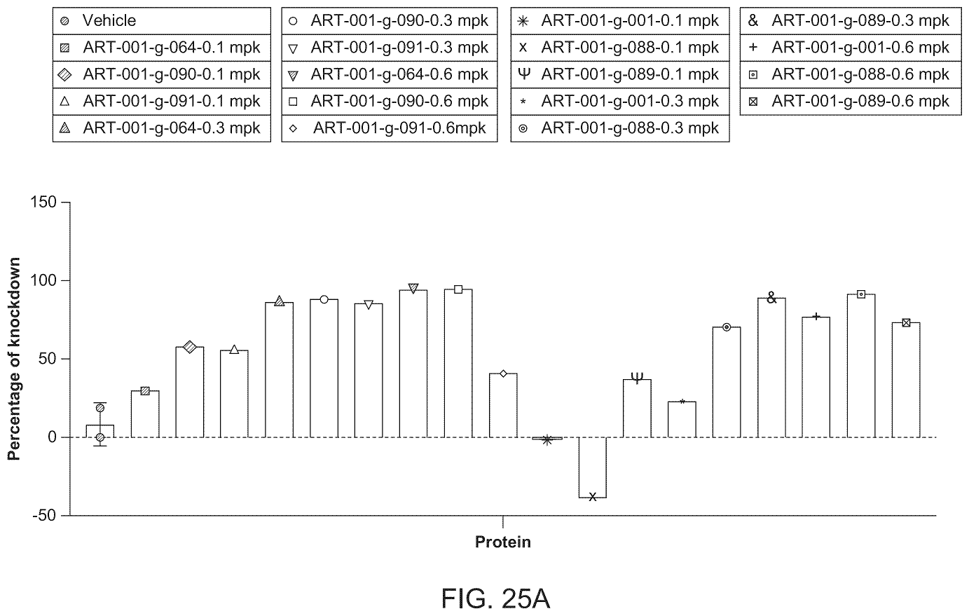

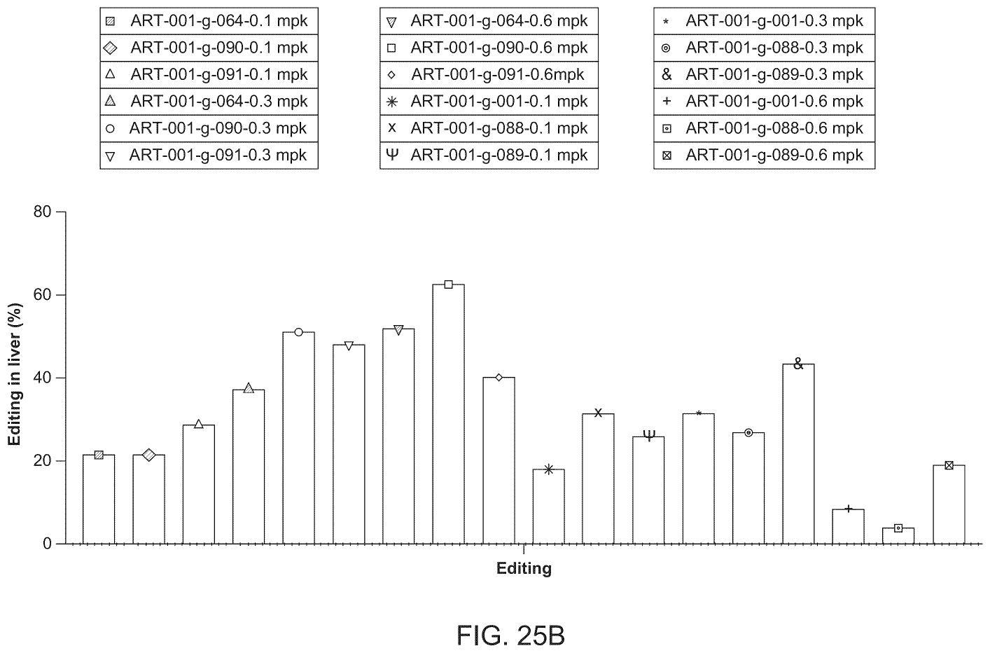

In an independent investigation, humanized mice were genetically modified to replace a region of the native murine Ttr locus with an orthologous, unmodified human TTR sequence. As a result, the locus was reprogrammed to produce human TTR protein. The LNP formulations were prepared using cross-flow and TFF procedures as described above containing DNA/RNA hybrid sgRNAs targeting human TTR and Cas9 mRNA in a 1:2 ratio by weight. The LNP contained ALC0315, DSPC, Cholesterol, and PEG2k-DMG in a 50:9.5:37.5:3 molar ratio, respectively, having an N:P ratio of 7. Dosing was at 0.1, 0.3, 0.6 mg/kg (n=1/group). Results for serum mouse TTR levels and liver editing are shown in FIG. 25A-B.

### OTHER EMBODIMENTS

It is to be understood that the foregoing description is intended to illustrate and not limit the scope of the appended claims. Other aspects, advantages, and modifications are within the scope of the following claims.

1. (canceled)

2. A guide RNA comprising:
a. a sequence selected from SEQ ID NOs: 302 or 303, SEQ ID NOs: 48-81 or SEQ ID NOs: 275-301 or 304-307;
b. at least 18, 19, or 20 contiguous nucleotides of a sequence selected from SEQ ID NOs: 302 or 303, SEQ ID NOs: 48-81 or SEQ ID NOs: 275-301 or 304-307; or
c. a sequence that is at least 90%, 91%, 92%, 93%, 94%, 95%, 96%, 97%, 98%, or 99% identical to a sequence selected from SEQ ID NOs: 302 or 303, SEQ ID NOs: 48-81 or SEQ ID NOs: 275-301 or 304-307.

3. A vector comprising one or more nucleic acids encoding one or more guide RNAs, wherein the one or more guide RNAs comprise:
a. one or more sequences selected from SEQ ID NOs: 302 or 303, SEQ ID NOs: 48-81 or SEQ ID NOs: 275-301 or 304-307;
b. at least 18, 19, or 20 contiguous nucleotides of one or more sequences selected from SEQ ID NOs: 302 or 303, SEQ ID NOs: 48-81 or SEQ ID NOs: 275-301 or 304-307; or
c. one or more sequences, each of which is at least 90%, 91%, 92%, 93%, 94%, 95%, 96%, 97%, 98%, or 99% identical to a sequence selected from SEQ ID NOs: 302 or 303, SEQ ID NOs: 48-81 or SEQ ID NOs: 275-301 or 304-307.

4. A composition comprising
(i) a nucleic acid encoding a guide RNA, or a vector comprising the nucleic acid encoding a guide RNA, wherein the guide RNA comprises a. a sequence selected from SEQ ID NOs: 302 or 303, SEQ ID NOs: 48-81 or SEQ ID NOs: 275-301 or 304-307; b. at least 18, 19, or 20 contiguous nucleotides of a sequence selected from SEQ ID NOs: 302 or 303, SEQ ID NOs: 48-81 or SEQ ID NOs: 275-301 or 304-307; or c. a sequence that is at least 90%, 91%, 92%, 93%, 94%, 95%, 96%, 97%, 98%, or 99% identical to a sequence selected from SEQ ID NOs: 302 or 303, SEQ ID NOs: 48-81 or SEQ ID NOs: 275-301 or 304-307; and
(ii) an RNA-guided DNA binding agent, a nucleic acid encoding an RNA-guided DNA binding agent, or a vector comprising the nucleic acid encoding an RNA-guided DNA binding agent.

5. The composition of claim 4, wherein the guide RNA is a hybrid DNA-RNA guide that comprises a sequence selected from SEQ ID NOs: 302 and 303, SEQ ID NOs: 48-81 or SEQ ID NOs: 275-301 or 304-307.

6. (canceled)

7. The composition of claim 4, wherein the RNA-guided DNA binding agent comprises a Cas nuclease or a Cas nickase.

8.-13. (canceled)

14. The composition of claim 7, wherein the Cas nuclease is fused to an exonuclease.

15.-34. (canceled)

35. The composition of claim 4, wherein the guide RNA is a dual guide RNA (dgRNA) or a single guide (sgRNA).

36. (canceled)

37. The composition of claim 4, wherein guide RNA comprises at least one modification.

38. The composition of claim 37, wherein the at least one modification comprises a 2′-O-methyl (2′-O-Me) modified nucleotide, a phosphorothioate (PS) bond between nucleotides, or a 2′-fluoro (2′-F) modified nucleotide.

39. The composition of claim 37, wherein the at least one modification comprises a modification at one or more of the first five nucleotides at the 5′ end of the guide RNA and/or one or more of the last five nucleotides at the 3′ end of the guide RNA.

40.-42. (canceled)

43. The composition of claim 35, wherein the sgRNA comprises a nucleotide sequence that is at least 90% identical to the nucleotide sequence set forth in any one of SEQ ID NOs: 368 or 369, SEQ ID NOs: 218-251 or SEQ ID NOs: 341-367 or 370-373.

44. (canceled)

45. The composition of claim 43, wherein the sgRNA comprises a nucleotide sequence set forth in any one of SEQ ID NOs: 368-369.

46. The composition of claim 4, wherein
(i) the nucleic acid encoding the guide RNA or the vector comprising the nucleic acid encoding the guide RNA, and
(ii) an mRNA encoding the RNA-guided DNA binding agent,
are associated with a lipid nanoparticle (LNP).

47. The composition of claim 45, wherein the LNP comprises ALC0315, DSPC, cholesterol, and DMG-PEG2000.

48. The composition of claim 46, wherein the N/P ratio of the LNP is about 1, 2, 3, 4, 5, 6, 7, 8, 9, or 10.

49. (canceled)

50. The composition of claim 47, wherein ALC0315 comprises from 40 mol % to 60 mol % of the total lipid present in the particle, DSPC comprises from 5 mol % to 15 mol % of the total lipid present in the particle, cholesterol comprises from 30 mol % to 50 mol % of the total lipid present in the particle, and DMG-PEG2000 comprises from 1 mol % to 5 mol % total lipid present in the particle.

51. (canceled)

52. The composition of claim 4, wherein the composition is a pharmaceutical formulation and further comprises a pharmaceutically acceptable carrier.

53.-73. (canceled)

74. A method of modifying the human transthyretin (TTR) gene and/or inducing a double-stranded break (DSB) within the TTR gene, comprising administering the composition of claim 4 to a cell, wherein the composition recognizes and cleaves a TTR target sequence.

75. A method of reducing TTR serum concentration, reducing or preventing the amyloid deposition or amyloid fibrils, and/or treating transthyretin amyloidosis (ATTR) in a subject, comprising administering the composition of claim 4 to the subject in need thereof, wherein the composition recognizes and cleaves a TTR target sequence, thereby reducing TTR serum concentration, reducing or preventing the deposition of amyloids or amyloid fibrils, and/or treating transthyretin amyloidosis (ATTR) in the subject.

76.-130. (canceled)

131. The method of claim 75, wherein the subject has ATTRwt, hereditary ATTR, a family history of ATTR, or familial amyloid polyneuropathy.

132.-139. (canceled)

140. The method of claim 75, wherein the composition is administered via a viral vector or via lipid nanoparticles.

141. (canceled)

142. A composition comprising:
(i) a hybrid DNA-RNA guide comprising a sequence selected from SEQ ID NO: 302 or 303; and
(ii) a Cas nuclease fused to an exonuclease;
wherein the hybrid DNA-RNA guide and the Cas nuclease fused to the exonuclease are associated with an LNP, wherein the LNP comprises ALC0315, DSPC, cholesterol, and DMG-PEG2000 at a molar ratio of 50:9.5:37.5:3.

143. A method of modifying the human transthyretin (TTR) gene and/or inducing a double-stranded break (DSB) within the TTR gene, comprising administering the composition of claim 142 to a human subject in need thereof.


SEQUENCE LISTING (374 sequences)

SEQ ID NO: 1 - RNA, 20 nt, synthetic construct
  source 1..20: mol_type = other RNA
cccattccatgagcatgcag

SEQ ID NO: 2 - RNA, 20 nt, synthetic construct
  source 1..20: mol_type = other RNA
gccatcctgccaagaatgag

SEQ ID NO: 3 - RNA, 20 nt, synthetic construct
  source 1..20: mol_type = other RNA
gaatgagtggacttctgtga

SEQ ID NO: 4 - RNA, 20 nt, synthetic construct
  source 1..20: mol_type = other RNA
tcctataaggtgtgaaagtc

SEQ ID NO: 5 - RNA, 20 nt, synthetic construct
  source 1..20: mol_type = other RNA
gtgagtatacagaccttcga

SEQ ID NO: 6 - RNA, 20 nt, synthetic construct
  source 1..20: mol_type = other RNA
ggtgagtatacagaccttcg

SEQ ID NO: 7 - RNA, 20 nt, synthetic construct
  source 1..20: mol_type = other RNA
atgagggacttctcctccag

SEQ ID NO: 8 - RNA, 20 nt, synthetic construct
  source 1..20: mol_type = other RNA
ttctcctccagtggacctga

SEQ ID NO: 9 - RNA, 20 nt, synthetic construct
  source 1..20: mol_type = other RNA
ccagtggacctgaaggacga

SEQ ID NO: 10 - RNA, 20 nt, synthetic construct
  source 1..20: mol_type = other RNA
tccagtggacctgaaggacg

SEQ ID NO: 11 - RNA, 20 nt, synthetic construct
  source 1..20: mol_type = other RNA
tcgtccttcaggtccactgg

SEQ ID NO: 12 - RNA, 20 nt, synthetic construct
  source 1..20: mol_type = other RNA
aatcccatccctcgtccttc

SEQ ID NO: 13 - RNA, 20 nt, synthetic construct
  source 1..20: mol_type = other RNA
ccctcgtccttcaggtccac

SEQ ID NO: 14 - RNA, 20 nt, synthetic construct
  source 1..20: mol_type = other RNA
tggacctgaaggacgaggga

SEQ ID NO: 15 - RNA, 20 nt, synthetic construct
  source 1..20: mol_type = other RNA
ggacctgaaggacgagggat

SEQ ID NO: 16 - RNA, 20 nt, synthetic construct
  source 1..20: mol_type = other RNA
tagtaaaaatggaatactct

SEQ ID NO: 17 - RNA, 20 nt, synthetic construct
  source 1..20: mol_type = other RNA
aaacactgctttagtaaaaa

SEQ ID NO: 18 - RNA, 20 nt, synthetic construct
  source 1..20: mol_type = other RNA
gacttctaacatagcatatg

SEQ ID NO: 19 - RNA, 20 nt, synthetic construct
  source 1..20: mol_type = other RNA
atatgctatgttagaagtcc

SEQ ID NO: 20 - RNA, 20 nt, synthetic construct
  source 1..20: mol_type = other RNA
aatgttttattgtctctgcc

SEQ ID NO: 21 - RNA, 20 nt, synthetic construct
  source 1..20: mol_type = other RNA
caataaaacattcctgtgaa

SEQ ID NO: 22 - RNA, 20 nt, synthetic construct
  source 1..20: mol_type = other RNA
gaatgaaaagtgcctttcac

SEQ ID NO: 23 - RNA, 20 nt, synthetic construct
  source 1..20: mol_type = other RNA
agtcaataatcagaatcagc

SEQ ID NO: 24 - RNA, 20 nt, synthetic construct
  source 1..20: mol_type = other RNA
cagcaggtttgcagtcagat

SEQ ID NO: 25 - RNA, 20 nt, synthetic construct
  source 1..20: mol_type = other RNA
aggtttgcagtcagattggc

SEQ ID NO: 26 - RNA, 20 nt, synthetic construct
  source 1..20: mol_type = other RNA
ggtttgcagtcagattggca

SEQ ID NO: 27 - RNA, 20 nt, synthetic construct
  source 1..20: mol_type = other RNA
agggataagcagcctagctc

SEQ ID NO: 28 - RNA, 20 nt, synthetic construct
  source 1..20: mol_type = other RNA
atactcacttctcctgagct

SEQ ID NO: 29 - RNA, 20 nt, synthetic construct
  source 1..20: mol_type = other RNA
gaagtgagtataaaagcccc

SEQ ID NO: 30 - RNA, 20 nt, synthetic construct
  source 1..20: mol_type = other RNA
tgagtataaaagccccaggc

SEQ ID NO: 31 - RNA, 20 nt, synthetic construct
  source 1..20: mol_type = other RNA
gagtataaaagccccaggct

SEQ ID NO: 32 - RNA, 20 nt, synthetic construct
  source 1..20: mol_type = other RNA
tgatggctgctcccagcctg

SEQ ID NO: 33 - RNA, 20 nt, synthetic construct
  source 1..20: mol_type = other RNA
gtgatggctgctcccagcct

SEQ ID NO: 34 - RNA, 20 nt, synthetic construct
  source 1..20: mol_type = other RNA
tgtgatggctgctcccagcc

SEQ ID NO: 35 - RNA, 20 nt, synthetic construct
  source 1..20: mol_type = other RNA
tttaaaaaatcaagttaaag

SEQ ID NO: 36 - RNA, 20 nt, synthetic construct
  source 1..20: mol_type = other RNA
ttttttggaagggacaataa

SEQ ID NO: 37 - RNA, 20 nt, synthetic construct
  source 1..20: mol_type = other RNA
tttttttggaagggacaata

SEQ ID NO: 38 - RNA, 20 nt, synthetic construct
  source 1..20: mol_type = other RNA
tgattctcttttttttggaa

SEQ ID NO: 39 - RNA, 20 nt, synthetic construct
  source 1..20: mol_type = other RNA
ttgattctcttttttttgga

SEQ ID NO: 40 - RNA, 20 nt, synthetic construct
  source 1..20: mol_type = other RNA
aattttgattctcttttttt

SEQ ID NO: 41 - RNA, 20 nt, synthetic construct
  source 1..20: mol_type = other RNA
aaaattttacaaagaatcaa

SEQ ID NO: 42 - RNA, 20 nt, synthetic construct
  source 1..20: mol_type = other RNA
aaggaattctagaaagtatc

SEQ ID NO: 43 - RNA, 20 nt, synthetic construct
  source 1..20: mol_type = other RNA
aggaattctagaaagtatct

SEQ ID NO: 44 - RNA, 20 nt, synthetic construct
  source 1..20: mol_type = other RNA
aagtatctgggcagaacgct

SEQ ID NO: 45 - RNA, 20 nt, synthetic construct
  source 1..20: mol_type = other RNA
cttgcaagacaatggaaatt

SEQ ID NO: 46 - RNA, 20 nt, synthetic construct
  source 1..20: mol_type = other RNA
gtgctttgcttgcaagacaa

SEQ ID NO: 47 - RNA, 20 nt, synthetic construct
  source 1..20: mol_type = other RNA
tacagaatgtgtctttttaa

SEQ ID NO: 48 - RNA, 20 nt, synthetic construct
  source 1..20: mol_type = other RNA
tgtgtctgaggctggcccta

SEQ ID NO: 49 - RNA, 20 nt, synthetic construct
  source 1..20: mol_type = other RNA
cttggattcaccggtgccct

SEQ ID NO: 50 - RNA, 20 nt, synthetic construct
  source 1..20: mol_type = other RNA
acttggattcaccggtgccc

SEQ ID NO: 51 - RNA, 20 nt, synthetic construct
  source 1..20: mol_type = other RNA
tccagactttcacaccttat

SEQ ID NO: 52 - RNA, 20 nt, synthetic construct
  source 1..20: mol_type = other RNA
gactcactggttttcctata

SEQ ID NO: 53 - RNA, 20 nt, synthetic construct
  source 1..20: mol_type = other RNA
atctgtctgtcttctctcat

SEQ ID NO: 54 - RNA, 20 nt, synthetic construct
  source 1..20: mol_type = other RNA
gtcaccaatcccaaggaatg

SEQ ID NO: 55 - RNA, 20 nt, synthetic construct
  source 1..20: mol_type = other RNA
tcaccaatcccaaggaatga

SEQ ID NO: 56 - RNA, 20 nt, synthetic construct
  source 1..20: mol_type = other RNA
aggagaagtccctcattcct

SEQ ID NO: 57 - RNA, 20 nt, synthetic construct
  source 1..20: mol_type = other RNA
ggagaagtccctcattcctt

SEQ ID NO: 58 - RNA, 20 nt, synthetic construct
  source 1..20: mol_type = other RNA
gaagtccactcattcttggc

SEQ ID NO: 59 - RNA, 20 nt, synthetic construct
  source 1..20: mol_type = other RNA
cacagaagtccactcattct

SEQ ID NO: 60 - RNA, 20 nt, synthetic construct
  source 1..20: mol_type = other RNA
gggagctgctgatgacacct

SEQ ID NO: 61 - RNA, 20 nt, synthetic construct
  source 1..20: mol_type = other RNA
aggagctgctgatgacacct

SEQ ID NO: 62 - RNA, 20 nt, synthetic construct
  source 1..20: mol_type = other RNA
ggaagctgctgatgacacct

SEQ ID NO: 63 - RNA, 20 nt, synthetic construct
  source 1..20: mol_type = other RNA
gagagctgctgatgacacct

SEQ ID NO: 64 - RNA, 20 nt, synthetic construct
  source 1..20: mol_type = other RNA
  modified_base 6: mod_base = OTHER; note = Thymine
agaagttgctgatgacacct

SEQ ID NO: 65 - RNA, 20 nt, synthetic construct
  source 1..20: mol_type = other RNA
gaggactgctgatgacacct

SEQ ID NO: 66 - RNA, 20 nt, synthetic construct
  source 1..20: mol_type = other RNA
ttttgctgctgatgacacct

SEQ ID NO: 67 - RNA, 20 nt, synthetic construct
  source 1..20: mol_type = other RNA
ccccgctgctgatgacacct

SEQ ID NO: 68 - RNA, 20 nt, synthetic construct
  source 1..20: mol_type = other RNA
ttccgctgctgatgacacct

SEQ ID NO: 69 - RNA, 20 nt, synthetic construct
  source 1..20: mol_type = other RNA
ccttgctgctgatgacacct

SEQ ID NO: 70 - RNA, 20 nt, synthetic construct
  source 1..20: mol_type = other RNA
tctcgctgctgatgacacct

SEQ ID NO: 71 - RNA, 20 nt, synthetic construct
  source 1..20: mol_type = other RNA
ctctgctgctgatgacacct

SEQ ID NO: 72 - RNA, 20 nt, synthetic construct
  source 1..20: mol_type = other RNA
tttcgctgctgatgacacct

SEQ ID NO: 73 - RNA, 20 nt, synthetic construct
  source 1..20: mol_type = other RNA
ccctgctgctgatgacacct

SEQ ID NO: 74 - RNA, 20 nt, synthetic construct
  source 1..20: mol_type = other RNA
ggccgctgctgatgacacct

SEQ ID NO: 75 - RNA, 20 nt, synthetic construct
  source 1..20: mol_type = other RNA
ggttgctgctgatgacacct

SEQ ID NO: 76 - RNA, 20 nt, synthetic construct
  source 1..20: mol_type = other RNA
gcgcgctgctgatgacacct

SEQ ID NO: 77 - RNA, 20 nt, synthetic construct
  source 1..20: mol_type = other RNA
gtgtgctgctgatgacacct

SEQ ID NO: 78 - RNA, 20 nt, synthetic construct
  source 1..20: mol_type = other RNA
gggcgctgctgatgacacct

SEQ ID NO: 79 - RNA, 20 nt, synthetic construct
  source 1..20: mol_type = other RNA
gggtgctgctgatgacacct

SEQ ID NO: 80 - RNA, 20 nt, synthetic construct
  source 1..20: mol_type = other RNA
gcccgctgctgatgacacct

SEQ ID NO: 81 - RNA, 20 nt, synthetic construct
  source 1..20: mol_type = other RNA
gtttgctgctgatgacacct

SEQ ID NO: 82 - RNA, 20 nt, synthetic construct
  source 1..20: mol_type = other RNA
aaagttctagatgctgtccg

SEQ ID NO: 83 - RNA, 20 nt, synthetic construct
  source 1..20: mol_type = other RNA
gggcggcaatggtgtagcgg

SEQ ID NO: 84 - RNA, 20 nt, synthetic construct
  source 1..20: mol_type = other RNA
cattgatggcaggactgcct

SEQ ID NO: 85 - RNA, 20 nt, synthetic construct
  source 1..20: mol_type = other RNA
aaaggctgctgatgacacct

SEQ ID NO: 86 - RNA, 42 nt, synthetic construct
  source 1..42: mol_type = other RNA
cccattccatgagcatgcaggttttagagctatgctgttttg

SEQ ID NO: 87 - RNA, 42 nt, synthetic construct
  source 1..42: mol_type = other RNA
gccatcctgccaagaatgaggttttagagctatgctgttttg

SEQ ID NO: 88 - RNA, 42 nt, synthetic construct
  source 1..42: mol_type = other RNA
gaatgagtggacttctgtgagttttagagctatgctgttttg

SEQ ID NO: 89 - RNA, 42 nt, synthetic construct
  source 1..42: mol_type = other RNA
tcctataaggtgtgaaagtcgttttagagctatgctgttttg

SEQ ID NO: 90 - RNA, 42 nt, synthetic construct
  source 1..42: mol_type = other RNA
gtgagtatacagaccttcgagttttagagctatgctgttttg

SEQ ID NO: 91 - RNA, 42 nt, synthetic construct
  source 1..42: mol_type = other RNA
ggtgagtatacagaccttcggttttagagctatgctgttttg

SEQ ID NO: 92 - RNA, 42 nt, synthetic construct
  source 1..42: mol_type = other RNA
atgagggacttctcctccaggttttagagctatgctgttttg

SEQ ID NO: 93 - RNA, 42 nt, synthetic construct
  source 1..42: mol_type = other RNA
ttctcctccagtggacctgagttttagagctatgctgttttg

SEQ ID NO: 94 - RNA, 42 nt, synthetic construct
  source 1..42: mol_type = other RNA
ccagtggacctgaaggacgagttttagagctatgctgttttg

SEQ ID NO: 95 - RNA, 42 nt, synthetic construct
  source 1..42: mol_type = other RNA
tccagtggacctgaaggacggttttagagctatgctgttttg

SEQ ID NO: 96 - RNA, 42 nt, synthetic construct
  source 1..42: mol_type = other RNA
tcgtccttcaggtccactgggttttagagctatgctgttttg

SEQ ID NO: 97 - RNA, 42 nt, synthetic construct
  source 1..42: mol_type = other RNA
aatcccatccctcgtccttcgttttagagctatgctgttttg

SEQ ID NO: 98 - RNA, 42 nt, synthetic construct
  source 1..42: mol_type = other RNA
ccctcgtccttcaggtccacgttttagagctatgctgttttg

SEQ ID NO: 99 - RNA, 42 nt, synthetic construct
  source 1..42: mol_type = other RNA
tggacctgaaggacgagggagttttagagctatgctgttttg

SEQ ID NO: 100 - RNA, 42 nt, synthetic construct
  source 1..42: mol_type = other RNA
ggacctgaaggacgagggatgttttagagctatgctgttttg

SEQ ID NO: 101 - RNA, 42 nt, synthetic construct
  source 1..42: mol_type = other RNA
tagtaaaaatggaatactctgttttagagctatgctgttttg

SEQ ID NO: 102 - RNA, 42 nt, synthetic construct
  source 1..42: mol_type = other RNA
aaacactgctttagtaaaaagttttagagctatgctgttttg

SEQ ID NO: 103 - RNA, 42 nt, synthetic construct
  source 1..42: mol_type = other RNA
gacttctaacatagcatatggttttagagctatgctgttttg

SEQ ID NO: 104 - RNA, 42 nt, synthetic construct
  source 1..42: mol_type = other RNA
atatgctatgttagaagtccgttttagagctatgctgttttg

SEQ ID NO: 105 - RNA, 42 nt, synthetic construct
  source 1..42: mol_type = other RNA
aatgttttattgtctctgccgttttagagctatgctgttttg

SEQ ID NO: 106 - RNA, 42 nt, synthetic construct
  source 1..42: mol_type = other RNA
caataaaacattcctgtgaagttttagagctatgctgttttg

SEQ ID NO: 107 - RNA, 42 nt, synthetic construct
  source 1..42: mol_type = other RNA
gaatgaaaagtgcctttcacgttttagagctatgctgttttg

SEQ ID NO: 108 - RNA, 42 nt, synthetic construct
  source 1..42: mol_type = other RNA
agtcaataatcagaatcagcgttttagagctatgctgttttg

SEQ ID NO: 109 - RNA, 42 nt, synthetic construct
  source 1..42: mol_type = other RNA
cagcaggtttgcagtcagatgttttagagctatgctgttttg

SEQ ID NO: 110 - RNA, 42 nt, synthetic construct
  source 1..42: mol_type = other RNA
aggtttgcagtcagattggcgttttagagctatgctgttttg

SEQ ID NO: 111 - RNA, 42 nt, synthetic construct
  source 1..42: mol_type = other RNA
ggtttgcagtcagattggcagttttagagctatgctgttttg

SEQ ID NO: 112 - RNA, 42 nt, synthetic construct
  source 1..42: mol_type = other RNA
agggataagcagcctagctcgttttagagctatgctgttttg

SEQ ID NO: 113 - RNA, 42 nt, synthetic construct
  source 1..42: mol_type = other RNA
atactcacttctcctgagctgttttagagctatgctgttttg

SEQ ID NO: 114 - RNA, 42 nt, synthetic construct
  source 1..42: mol_type = other RNA
gaagtgagtataaaagccccgttttagagctatgctgttttg

SEQ ID NO: 115 - RNA, 42 nt, synthetic construct
  source 1..42: mol_type = other RNA
tgagtataaaagccccaggcgttttagagctatgctgttttg

SEQ ID NO: 116 - RNA, 42 nt, synthetic construct
  source 1..42: mol_type = other RNA
gagtataaaagccccaggctgttttagagctatgctgttttg

SEQ ID NO: 117 - RNA, 42 nt, synthetic construct
  source 1..42: mol_type = other RNA
tgatggctgctcccagcctggttttagagctatgctgttttg

SEQ ID NO: 118 - RNA, 42 nt, synthetic construct
  source 1..42: mol_type = other RNA
gtgatggctgctcccagcctgttttagagctatgctgttttg

SEQ ID NO: 119 - RNA, 42 nt, synthetic construct
  source 1..42: mol_type = other RNA
tgtgatggctgctcccagccgttttagagctatgctgttttg

SEQ ID NO: 120 - RNA, 42 nt, synthetic construct
  source 1..42: mol_type = other RNA
tttaaaaaatcaagttaaaggttttagagctatgctgttttg

SEQ ID NO: 121 - RNA, 42 nt, synthetic construct
  source 1..42: mol_type = other RNA
ttttttggaagggacaataagttttagagctatgctgttttg

SEQ ID NO: 122 - RNA, 42 nt, synthetic construct
  source 1..42: mol_type = other RNA
tttttttggaagggacaatagttttagagctatgctgttttg

SEQ ID NO: 123 - RNA, 42 nt, synthetic construct
  source 1..42: mol_type = other RNA
tgattctcttttttttggaagttttagagctatgctgttttg

SEQ ID NO: 124 - RNA, 42 nt, synthetic construct
  source 1..42: mol_type = other RNA
ttgattctcttttttttggagttttagagctatgctgttttg

SEQ ID NO: 125 - RNA, 42 nt, synthetic construct
  source 1..42: mol_type = other RNA
aattttgattctctttttttgttttagagctatgctgttttg

SEQ ID NO: 126 - RNA, 42 nt, synthetic construct
  source 1..42: mol_type = other RNA
aaaattttacaaagaatcaagttttagagctatgctgttttg

SEQ ID NO: 127 - RNA, 42 nt, synthetic construct
  source 1..42: mol_type = other RNA
aaggaattctagaaagtatcgttttagagctatgctgttttg

SEQ ID NO: 128 - RNA, 42 nt, synthetic construct
  source 1..42: mol_type = other RNA
aggaattctagaaagtatctgttttagagctatgctgttttg

SEQ ID NO: 129 - RNA, 42 nt, synthetic construct
  source 1..42: mol_type = other RNA
aagtatctgggcagaacgctgttttagagctatgctgttttg

SEQ ID NO: 130 - RNA, 42 nt, synthetic construct
  source 1..42: mol_type = other RNA
cttgcaagacaatggaaattgttttagagctatgctgttttg

SEQ ID NO: 131 - RNA, 42 nt, synthetic construct
  source 1..42: mol_type = other RNA
gtgctttgcttgcaagacaagttttagagctatgctgttttg

SEQ ID NO: 132 - RNA, 42 nt, synthetic construct
  source 1..42: mol_type = other RNA
tacagaatgtgtctttttaagttttagagctatgctgttttg

SEQ ID NO: 133 - RNA, 42 nt, synthetic construct
  source 1..42: mol_type = other RNA
tgtgtctgaggctggccctagttttagagctatgctgttttg

SEQ ID NO: 134 - RNA, 42 nt, synthetic construct
  source 1..42: mol_type = other RNA
cttggattcaccggtgccctgttttagagctatgctgttttg

SEQ ID NO: 135 - RNA, 42 nt, synthetic construct
  source 1..42: mol_type = other RNA
acttggattcaccggtgcccgttttagagctatgctgttttg

SEQ ID NO: 136 - RNA, 42 nt, synthetic construct
  source 1..42: mol_type = other RNA
tccagactttcacaccttatgttttagagctatgctgttttg

SEQ ID NO: 137 - RNA, 42 nt, synthetic construct
  source 1..42: mol_type = other RNA
gactcactggttttcctatagttttagagctatgctgttttg

SEQ ID NO: 138 - RNA, 42 nt, synthetic construct
  source 1..42: mol_type = other RNA
atctgtctgtcttctctcatgttttagagctatgctgttttg

SEQ ID NO: 139 - RNA, 42 nt, synthetic construct
  source 1..42: mol_type = other RNA
gtcaccaatcccaaggaatggttttagagctatgctgttttg

SEQ ID NO: 140 - RNA, 42 nt, synthetic construct
  source 1..42: mol_type = other RNA
tcaccaatcccaaggaatgagttttagagctatgctgttttg

SEQ ID NO: 141 - RNA, 42 nt, synthetic construct
  source 1..42: mol_type = other RNA
aggagaagtccctcattcctgttttagagctatgctgttttg

SEQ ID NO: 142 - RNA, 42 nt, synthetic construct
  source 1..42: mol_type = other RNA
ggagaagtccctcattccttgttttagagctatgctgttttg

SEQ ID NO: 143 - RNA, 42 nt, synthetic construct
  source 1..42: mol_type = other RNA
gaagtccactcattcttggcgttttagagctatgctgttttg

SEQ ID NO: 144 - RNA, 42 nt, synthetic construct
  source 1..42: mol_type = other RNA
cacagaagtccactcattctgttttagagctatgctgttttg

SEQ ID NO: 145 - RNA, 42 nt, synthetic construct
  source 1..42: mol_type = other RNA
gggagctgctgatgacacctgttttagagctatgctgttttg

SEQ ID NO: 146 - RNA, 42 nt, synthetic construct
  source 1..42: mol_type = other RNA
aggagctgctgatgacacctgttttagagctatgctgttttg

SEQ ID NO: 147 - RNA, 42 nt, synthetic construct
  source 1..42: mol_type = other RNA
ggaagctgctgatgacacctgttttagagctatgctgttttg

SEQ ID NO: 148 - RNA, 42 nt, synthetic construct
  source 1..42: mol_type = other RNA
gagagctgctgatgacacctgttttagagctatgctgttttg

SEQ ID NO: 149 - RNA, 42 nt, synthetic construct
  source 1..42: mol_type = other RNA
  modified_base 6: mod_base = OTHER; note = Thymine
agaagttgctgatgacacctgttttagagctatgctgttttg

SEQ ID NO: 150 - RNA, 42 nt, synthetic construct
  source 1..42: mol_type = other RNA
gaggactgctgatgacacctgttttagagctatgctgttttg

SEQ ID NO: 151 - RNA, 42 nt, synthetic construct
  source 1..42: mol_type = other RNA
ttttgctgctgatgacacctgttttagagctatgctgttttg

SEQ ID NO: 152 - RNA, 42 nt, synthetic construct
  source 1..42: mol_type = other RNA
ccccgctgctgatgacacctgttttagagctatgctgttttg

SEQ ID NO: 153 - RNA, 42 nt, synthetic construct
  source 1..42: mol_type = other RNA
ttccgctgctgatgacacctgttttagagctatgctgttttg

SEQ ID NO: 154 - RNA, 42 nt, synthetic construct
  source 1..42: mol_type = other RNA
ccttgctgctgatgacacctgttttagagctatgctgttttg

SEQ ID NO: 155 - RNA, 42 nt, synthetic construct
  source 1..42: mol_type = other RNA
tctcgctgctgatgacacctgttttagagctatgctgttttg

SEQ ID NO: 156 - RNA, 42 nt, synthetic construct
  source 1..42: mol_type = other RNA
ctctgctgctgatgacacctgttttagagctatgctgttttg

SEQ ID NO: 157 - RNA, 42 nt, synthetic construct
  source 1..42: mol_type = other RNA
tttcgctgctgatgacacctgttttagagctatgctgttttg

SEQ ID NO: 158 - RNA, 42 nt, synthetic construct
  source 1..42: mol_type = other RNA
ccctgctgctgatgacacctgttttagagctatgctgttttg

SEQ ID NO: 159 - RNA, 42 nt, synthetic construct
  source 1..42: mol_type = other RNA
ggccgctgctgatgacacctgttttagagctatgctgttttg

SEQ ID NO: 160 - RNA, 42 nt, synthetic construct
  source 1..42: mol_type = other RNA
ggttgctgctgatgacacctgttttagagctatgctgttttg

SEQ ID NO: 161 - RNA, 42 nt, synthetic construct
  source 1..42: mol_type = other RNA
gcgcgctgctgatgacacctgttttagagctatgctgttttg

SEQ ID NO: 162 - RNA, 42 nt, synthetic construct
  source 1..42: mol_type = other RNA
gtgtgctgctgatgacacctgttttagagctatgctgttttg

SEQ ID NO: 163 - RNA, 42 nt, synthetic construct
  source 1..42: mol_type = other RNA
gggcgctgctgatgacacctgttttagagctatgctgttttg

SEQ ID NO: 164 - RNA, 42 nt, synthetic construct
  source 1..42: mol_type = other RNA
gggtgctgctgatgacacctgttttagagctatgctgttttg

SEQ ID NO: 165 - RNA, 42 nt, synthetic construct
  source 1..42: mol_type = other RNA
gcccgctgctgatgacacctgttttagagctatgctgttttg

SEQ ID NO: 166 - RNA, 42 nt, synthetic construct
  source 1..42: mol_type = other RNA
gtttgctgctgatgacacctgttttagagctatgctgttttg

SEQ ID NO: 167 - RNA, 42 nt, synthetic construct
  source 1..42: mol_type = other RNA
aaagttctagatgctgtccggttttagagctatgctgttttg

SEQ ID NO: 168 - RNA, 42 nt, synthetic construct
  source 1..42: mol_type = other RNA
gggcggcaatggtgtagcgggttttagagctatgctgttttg

SEQ ID NO: 169 - RNA, 42 nt, synthetic construct
  source 1..42: mol_type = other RNA
cattgatggcaggactgcctgttttagagctatgctgttttg

SEQ ID NO: 170 - RNA, 42 nt, synthetic construct
  source 1..42: mol_type = other RNA
aaaggctgctgatgacacctgttttagagctatgctgttttg

SEQ ID NO: 171 - RNA, 100 nt, synthetic construct
  source 1..100: mol_type = other RNA
cccattccatgagcatgcaggttttagagctagaaatagcaagttaaaataaggctagtccgttatcaacttgaaaaagtggcaccgagtcggtgctttt

SEQ ID NO: 172 - RNA, 100 nt, synthetic construct
  source 1..100: mol_type = other RNA
gccatcctgccaagaatgaggttttagagctagaaatagcaagttaaaataaggctagtccgttatcaacttgaaaaagtggcaccgagtcggtgctttt

SEQ ID NO: 173 - RNA, 100 nt, synthetic construct
  source 1..100: mol_type = other RNA
gaatgagtggacttctgtgagttttagagctagaaatagcaagttaaaataaggctagtccgttatcaacttgaaaaagtggcaccgagtcggtgctttt

SEQ ID NO: 174 - RNA, 100 nt, synthetic construct
  source 1..100: mol_type = other RNA
tcctataaggtgtgaaagtcgttttagagctagaaatagcaagttaaaataaggctagtccgttatcaacttgaaaaagtggcaccgagtcggtgctttt

SEQ ID NO: 175 - RNA, 100 nt, synthetic construct
  source 1..100: mol_type = other RNA
gtgagtatacagaccttcgagttttagagctagaaatagcaagttaaaataaggctagtccgttatcaacttgaaaaagtggcaccgagtcggtgctttt

SEQ ID NO: 176 - RNA, 100 nt, synthetic construct
  source 1..100: mol_type = other RNA
ggtgagtatacagaccttcggttttagagctagaaatagcaagttaaaataaggctagtccgttatcaacttgaaaaagtggcaccgagtcggtgctttt

SEQ ID NO: 177 - RNA, 100 nt, synthetic construct
  source 1..100: mol_type = other RNA
atgagggacttctcctccaggttttagagctagaaatagcaagttaaaataaggctagtccgttatcaacttgaaaaagtggcaccgagtcggtgctttt

SEQ ID NO: 178 - RNA, 100 nt, synthetic construct
  source 1..100: mol_type = other RNA
ttctcctccagtggacctgagttttagagctagaaatagcaagttaaaataaggctagtccgttatcaacttgaaaaagtggcaccgagtcggtgctttt

SEQ ID NO: 179 - RNA, 100 nt, synthetic construct
  source 1..100: mol_type = other RNA
ccagtggacctgaaggacgagttttagagctagaaatagcaagttaaaataaggctagtccgttatcaacttgaaaaagtggcaccgagtcggtgctttt

SEQ ID NO: 180 - RNA, 100 nt, synthetic construct
  source 1..100: mol_type = other RNA
tccagtggacctgaaggacggttttagagctagaaatagcaagttaaaataaggctagtccgttatcaacttgaaaaagtggcaccgagtcggtgctttt

SEQ ID NO: 181 - RNA, 100 nt, synthetic construct
  source 1..100: mol_type = other RNA
tcgtccttcaggtccactgggttttagagctagaaatagcaagttaaaataaggctagtccgttatcaacttgaaaaagtggcaccgagtcggtgctttt

SEQ ID NO: 182 - RNA, 100 nt, synthetic construct
  source 1..100: mol_type = other RNA
aatcccatccctcgtccttcgttttagagctagaaatagcaagttaaaataaggctagtccgttatcaacttgaaaaagtggcaccgagtcggtgctttt

SEQ ID NO: 183 - RNA, 100 nt, synthetic construct
  source 1..100: mol_type = other RNA
ccctcgtccttcaggtccacgttttagagctagaaatagcaagttaaaataaggctagtccgttatcaacttgaaaaagtggcaccgagtcggtgctttt

SEQ ID NO: 184 - RNA, 100 nt, synthetic construct
  source 1..100: mol_type = other RNA
tggacctgaaggacgagggagttttagagctagaaatagcaagttaaaataaggctagtccgttatcaacttgaaaaagtggcaccgagtcggtgctttt

SEQ ID NO: 185 - RNA, 100 nt, synthetic construct
  source 1..100: mol_type = other RNA
ggacctgaaggacgagggatgttttagagctagaaatagcaagttaaaataaggctagtccgttatcaacttgaaaaagtggcaccgagtcggtgctttt

SEQ ID NO: 186 - RNA, 100 nt, synthetic construct
  source 1..100: mol_type = other RNA
tagtaaaaatggaatactctgttttagagctagaaatagcaagttaaaataaggctagtccgttatcaacttgaaaaagtggcaccgagtcggtgctttt

SEQ ID NO: 187 - RNA, 100 nt, synthetic construct
  source 1..100: mol_type = other RNA
aaacactgctttagtaaaaagttttagagctagaaatagcaagttaaaataaggctagtccgttatcaacttgaaaaagtggcaccgagtcggtgctttt

SEQ ID NO: 188 - RNA, 100 nt, synthetic construct
  source 1..100: mol_type = other RNA
gacttctaacatagcatatggttttagagctagaaatagcaagttaaaataaggctagtccgttatcaacttgaaaaagtggcaccgagtcggtgctttt

SEQ ID NO: 189 - RNA, 100 nt, synthetic construct
  source 1..100: mol_type = other RNA
atatgctatgttagaagtccgttttagagctagaaatagcaagttaaaataaggctagtccgttatcaacttgaaaaagtggcaccgagtcggtgctttt

SEQ ID NO: 190 - RNA, 100 nt, synthetic construct
  source 1..100: mol_type = other RNA
aatgttttattgtctctgccgttttagagctagaaatagcaagttaaaataaggctagtccgttatcaacttgaaaaagtggcaccgagtcggtgctttt

SEQ ID NO: 191 - RNA, 100 nt, synthetic construct
  source 1..100: mol_type = other RNA
caataaaacattcctgtgaagttttagagctagaaatagcaagttaaaataaggctagtccgttatcaacttgaaaaagtggcaccgagtcggtgctttt

SEQ ID NO: 192 - RNA, 100 nt, synthetic construct
  source 1..100: mol_type = other RNA
gaatgaaaagtgcctttcacgttttagagctagaaatagcaagttaaaataaggctagtccgttatcaacttgaaaaagtggcaccgagtcggtgctttt

SEQ ID NO: 193 - RNA, 100 nt, synthetic construct
  source 1..100: mol_type = other RNA
agtcaataatcagaatcagcgttttagagctagaaatagcaagttaaaataaggctagtccgttatcaacttgaaaaagtggcaccgagtcggtgctttt

SEQ ID NO: 194 - RNA, 100 nt, synthetic construct
  source 1..100: mol_type = other RNA
cagcaggtttgcagtcagatgttttagagctagaaatagcaagttaaaataaggctagtccgttatcaacttgaaaaagtggcaccgagtcggtgctttt

SEQ ID NO: 195 - RNA, 100 nt, synthetic construct
  source 1..100: mol_type = other RNA
aggtttgcagtcagattggcgttttagagctagaaatagcaagttaaaataaggctagtccgttatcaacttgaaaaagtggcaccgagtcggtgctttt

SEQ ID NO: 196 - RNA, 100 nt, synthetic construct
  source 1..100: mol_type = other RNA
ggtttgcagtcagattggcagttttagagctagaaatagcaagttaaaataaggctagtccgttatcaacttgaaaaagtggcaccgagtcggtgctttt

SEQ ID NO: 197 - RNA, 100 nt, synthetic construct
  source 1..100: mol_type = other RNA
agggataagcagcctagctcgttttagagctagaaatagcaagttaaaataaggctagtccgttatcaacttgaaaaagtggcaccgagtcggtgctttt

SEQ ID NO: 198 - RNA, 100 nt, synthetic construct
  source 1..100: mol_type = other RNA
atactcacttctcctgagctgttttagagctagaaatagcaagttaaaataaggctagtccgttatcaacttgaaaaagtggcaccgagtcggtgctttt

SEQ ID NO: 199 - RNA, 100 nt, synthetic construct
  source 1..100: mol_type = other RNA
gaagtgagtataaaagccccgttttagagctagaaatagcaagttaaaataaggctagtccgttatcaacttgaaaaagtggcaccgagtcggtgctttt

SEQ ID NO: 200 - RNA, 100 nt, synthetic construct
  source 1..100: mol_type = other RNA
tgagtataaaagccccaggcgttttagagctagaaatagcaagttaaaataaggctagtccgttatcaacttgaaaaagtggcaccgagtcggtgctttt

SEQ ID NO: 201 - RNA, 100 nt, synthetic construct
  source 1..100: mol_type = other RNA
gagtataaaagccccaggctgttttagagctagaaatagcaagttaaaataaggctagtccgttatcaacttgaaaaagtggcaccgagtcggtgctttt

SEQ ID NO: 202 - RNA, 100 nt, synthetic construct
  source 1..100: mol_type = other RNA
tgatggctgctcccagcctggttttagagctagaaatagcaagttaaaataaggctagtccgttatcaacttgaaaaagtggcaccgagtcggtgctttt

SEQ ID NO: 203 - RNA, 100 nt, synthetic construct
  source 1..100: mol_type = other RNA
gtgatggctgctcccagcctgttttagagctagaaatagcaagttaaaataaggctagtccgttatcaacttgaaaaagtggcaccgagtcggtgctttt

SEQ ID NO: 204 - RNA, 100 nt, synthetic construct
  source 1..100: mol_type = other RNA
tgtgatggctgctcccagccgttttagagctagaaatagcaagttaaaataaggctagtccgttatcaacttgaaaaagtggcaccgagtcggtgctttt

SEQ ID NO: 205 - RNA, 100 nt, synthetic construct
  source 1..100: mol_type = other RNA
tttaaaaaatcaagttaaaggttttagagctagaaatagcaagttaaaataaggctagtccgttatcaacttgaaaaagtggcaccgagtcggtgctttt

SEQ ID NO: 206 - RNA, 100 nt, synthetic construct
  source 1..100: mol_type = other RNA
ttttttggaagggacaataagttttagagctagaaatagcaagttaaaataaggctagtccgttatcaacttgaaaaagtggcaccgagtcggtgctttt

SEQ ID NO: 207 - RNA, 100 nt, synthetic construct
  source 1..100: mol_type = other RNA
tttttttggaagggacaatagttttagagctagaaatagcaagttaaaataaggctagtccgttatcaacttgaaaaagtggcaccgagtcggtgctttt

SEQ ID NO: 208 - RNA, 100 nt, synthetic construct
  source 1..100: mol_type = other RNA
tgattctcttttttttggaagttttagagctagaaatagcaagttaaaataaggctagtccgttatcaacttgaaaaagtggcaccgagtcggtgctttt

SEQ ID NO: 209 - RNA, 100 nt, synthetic construct
  source 1..100: mol_type = other RNA
ttgattctcttttttttggagttttagagctagaaatagcaagttaaaataaggctagtccgttatcaacttgaaaaagtggcaccgagtcggtgctttt

SEQ ID NO: 210 - RNA, 100 nt, synthetic construct
  source 1..100: mol_type = other RNA
aattttgattctctttttttgttttagagctagaaatagcaagttaaaataaggctagtccgttatcaacttgaaaaagtggcaccgagtcggtgctttt

SEQ ID NO: 211 - RNA, 100 nt, synthetic construct
  source 1..100: mol_type = other RNA
aaaattttacaaagaatcaagttttagagctagaaatagcaagttaaaataaggctagtccgttatcaacttgaaaaagtggcaccgagtcggtgctttt

SEQ ID NO: 212 - RNA, 100 nt, synthetic construct
  source 1..100: mol_type = other RNA
aaggaattctagaaagtatcgttttagagctagaaatagcaagttaaaataaggctagtccgttatcaacttgaaaaagtggcaccgagtcggtgctttt

SEQ ID NO: 213 - RNA, 100 nt, synthetic construct
  source 1..100: mol_type = other RNA
aggaattctagaaagtatctgttttagagctagaaatagcaagttaaaataaggctagtccgttatcaacttgaaaaagtggcaccgagtcggtgctttt

SEQ ID NO: 214 - RNA, 100 nt, synthetic construct
  source 1..100: mol_type = other RNA
aagtatctgggcagaacgctgttttagagctagaaatagcaagttaaaataaggctagtccgttatcaacttgaaaaagtggcaccgagtcggtgctttt

SEQ ID NO: 215 - RNA, 100 nt, synthetic construct
  source 1..100: mol_type = other RNA
cttgcaagacaatggaaattgttttagagctagaaatagcaagttaaaataaggctagtccgttatcaacttgaaaaagtggcaccgagtcggtgctttt

SEQ ID NO: 216 - RNA, 100 nt, synthetic construct
  source 1..100: mol_type = other RNA
gtgctttgcttgcaagacaagttttagagctagaaatagcaagttaaaataaggctagtccgttatcaacttgaaaaagtggcaccgagtcggtgctttt

SEQ ID NO: 217 - RNA, 100 nt, synthetic construct
  source 1..100: mol_type = other RNA
tacagaatgtgtctttttaagttttagagctagaaatagcaagttaaaataaggctagtccgttatcaacttgaaaaagtggcaccgagtcggtgctttt

SEQ ID NO: 218 - RNA, 100 nt, synthetic construct
  source 1..100: mol_type = other RNA
tgtgtctgaggctggccctagttttagagctagaaatagcaagttaaaataaggctagtccgttatcaacttgaaaaagtggcaccgagtcggtgctttt

SEQ ID NO: 219 - RNA, 100 nt, synthetic construct
  source 1..100: mol_type = other RNA
cttggattcaccggtgccctgttttagagctagaaatagcaagttaaaataaggctagtccgttatcaacttgaaaaagtggcaccgagtcggtgctttt

SEQ ID NO: 220 - RNA, 100 nt, synthetic construct
  source 1..100: mol_type = other RNA
acttggattcaccggtgcccgttttagagctagaaatagcaagttaaaataaggctagtccgttatcaacttgaaaaagtggcaccgagtcggtgctttt

SEQ ID NO: 221 - RNA, 100 nt, synthetic construct
  source 1..100: mol_type = other RNA
tccagactttcacaccttatgttttagagctagaaatagcaagttaaaataaggctagtccgttatcaacttgaaaaagtggcaccgagtcggtgctttt

SEQ ID NO: 222 - RNA, 100 nt, synthetic construct
  source 1..100: mol_type = other RNA
gactcactggttttcctatagttttagagctagaaatagcaagttaaaataaggctagtccgttatcaacttgaaaaagtggcaccgagtcggtgctttt

SEQ ID NO: 223 - RNA, 100 nt, synthetic construct
  source 1..100: mol_type = other RNA
atctgtctgtcttctctcatgttttagagctagaaatagcaagttaaaataaggctagtccgttatcaacttgaaaaagtggcaccgagtcggtgctttt

SEQ ID NO: 224 - RNA, 100 nt, synthetic construct
  source 1..100: mol_type = other RNA
gtcaccaatcccaaggaatggttttagagctagaaatagcaagttaaaataaggctagtccgttatcaacttgaaaaagtggcaccgagtcggtgctttt

SEQ ID NO: 225 - RNA, 100 nt, synthetic construct
  source 1..100: mol_type = other RNA
tcaccaatcccaaggaatgagttttagagctagaaatagcaagttaaaataaggctagtccgttatcaacttgaaaaagtggcaccgagtcggtgctttt

SEQ ID NO: 226 - RNA, 100 nt, synthetic construct
  source 1..100: mol_type = other RNA
aggagaagtccctcattcctgttttagagctagaaatagcaagttaaaataaggctagtccgttatcaacttgaaaaagtggcaccgagtcggtgctttt

SEQ ID NO: 227 - RNA, 100 nt, synthetic construct
  source 1..100: mol_type = other RNA
ggagaagtccctcattccttgttttagagctagaaatagcaagttaaaataaggctagtccgttatcaacttgaaaaagtggcaccgagtcggtgctttt

SEQ ID NO: 228 - RNA, 100 nt, synthetic construct
  source 1..100: mol_type = other RNA
gaagtccactcattcttggcgttttagagctagaaatagcaagttaaaataaggctagtccgttatcaacttgaaaaagtggcaccgagtcggtgctttt

SEQ ID NO: 229 - RNA, 100 nt, synthetic construct
  source 1..100: mol_type = other RNA
cacagaagtccactcattctgttttagagctagaaatagcaagttaaaataaggctagtccgttatcaacttgaaaaagtggcaccgagtcggtgctttt

SEQ ID NO: 230 - RNA, 100 nt, synthetic construct
  source 1..100: mol_type = other RNA
gggagctgctgatgacacctgttttagagctagaaatagcaagttaaaataaggctagtccgttatcaacttgaaaaagtggcaccgagtcggtgctttt

SEQ ID NO: 231 - RNA, 100 nt, synthetic construct
  source 1..100: mol_type = other RNA
aggagctgctgatgacacctgttttagagctagaaatagcaagttaaaataaggctagtccgttatcaacttgaaaaagtggcaccgagtcggtgctttt

SEQ ID NO: 232 - RNA, 100 nt, synthetic construct
  source 1..100: mol_type = other RNA
ggaagctgctgatgacacctgttttagagctagaaatagcaagttaaaataaggctagtccgttatcaacttgaaaaagtggcaccgagtcggtgctttt

SEQ ID NO: 233 - RNA, 100 nt, synthetic construct
  source 1..100: mol_type = other RNA
gagagctgctgatgacacctgttttagagctagaaatagcaagttaaaataaggctagtccgttatcaacttgaaaaagtggcaccgagtcggtgctttt

SEQ ID NO: 234 - RNA, 100 nt, synthetic construct
  source 1..100: mol_type = other RNA
  modified_base 6: mod_base = OTHER; note = Thymine
agaagttgctgatgacacctgttttagagctagaaatagcaagttaaaataaggctagtccgttatcaacttgaaaaagtggcaccgagtcggtgctttt

SEQ ID NO: 235 - RNA, 100 nt, synthetic construct
  source 1..100: mol_type = other RNA
gaggactgctgatgacacctgttttagagctagaaatagcaagttaaaataaggctagtccgttatcaacttgaaaaagtggcaccgagtcggtgctttt

SEQ ID NO: 236 - RNA, 100 nt, synthetic construct
  source 1..100: mol_type = other RNA
ttttgctgctgatgacacctgttttagagctagaaatagcaagttaaaataaggctagtccgttatcaacttgaaaaagtggcaccgagtcggtgctttt

SEQ ID NO: 237 - RNA, 100 nt, synthetic construct
  source 1..100: mol_type = other RNA
ccccgctgctgatgacacctgttttagagctagaaatagcaagttaaaataaggctagtccgttatcaacttgaaaaagtggcaccgagtcggtgctttt

SEQ ID NO: 238 - RNA, 100 nt, synthetic construct
  source 1..100: mol_type = other RNA
ttccgctgctgatgacacctgttttagagctagaaatagcaagttaaaataaggctagtccgttatcaacttgaaaaagtggcaccgagtcggtgctttt

SEQ ID NO: 239 - RNA, 100 nt, synthetic construct
  source 1..100: mol_type = other RNA
ccttgctgctgatgacacctgttttagagctagaaatagcaagttaaaataaggctagtccgttatcaacttgaaaaagtggcaccgagtcggtgctttt

SEQ ID NO: 240 - RNA, 100 nt, synthetic construct
  source 1..100: mol_type = other RNA
tctcgctgctgatgacacctgttttagagctagaaatagcaagttaaaataaggctagtccgttatcaacttgaaaaagtggcaccgagtcggtgctttt

SEQ ID NO: 241 - RNA, 100 nt, synthetic construct
  source 1..100: mol_type = other RNA
ctctgctgctgatgacacctgttttagagctagaaatagcaagttaaaataaggctagtccgttatcaacttgaaaaagtggcaccgagtcggtgctttt

SEQ ID NO: 242 - RNA, 100 nt, synthetic construct
  source 1..100: mol_type = other RNA
tttcgctgctgatgacacctgttttagagctagaaatagcaagttaaaataaggctagtccgttatcaacttgaaaaagtggcaccgagtcggtgctttt

SEQ ID NO: 243 - RNA, 100 nt, synthetic construct
  source 1..100: mol_type = other RNA
ccctgctgctgatgacacctgttttagagctagaaatagcaagttaaaataaggctagtccgttatcaacttgaaaaagtggcaccgagtcggtgctttt

SEQ ID NO: 244 - RNA, 100 nt, synthetic construct
  source 1..100: mol_type = other RNA
ggccgctgctgatgacacctgttttagagctagaaatagcaagttaaaataaggctagtccgttatcaacttgaaaaagtggcaccgagtcggtgctttt

SEQ ID NO: 245 - RNA, 100 nt, synthetic construct
  source 1..100: mol_type = other RNA
ggttgctgctgatgacacctgttttagagctagaaatagcaagttaaaataaggctagtccgttatcaacttgaaaaagtggcaccgagtcggtgctttt

SEQ ID NO: 246 - RNA, 100 nt, synthetic construct
  source 1..100: mol_type = other RNA
gcgcgctgctgatgacacctgttttagagctagaaatagcaagttaaaataaggctagtccgttatcaacttgaaaaagtggcaccgagtcggtgctttt

SEQ ID NO: 247 - RNA, 100 nt, synthetic construct
  source 1..100: mol_type = other RNA
gtgtgctgctgatgacacctgttttagagctagaaatagcaagttaaaataaggctagtccgttatcaacttgaaaaagtggcaccgagtcggtgctttt

SEQ ID NO: 248 - RNA, 100 nt, synthetic construct
  source 1..100: mol_type = other RNA
gggcgctgctgatgacacctgttttagagctagaaatagcaagttaaaataaggctagtccgttatcaacttgaaaaagtggcaccgagtcggtgctttt

SEQ ID NO: 249 - RNA, 100 nt, synthetic construct
  source 1..100: mol_type = other RNA
gggtgctgctgatgacacctgttttagagctagaaatagcaagttaaaataaggctagtccgttatcaacttgaaaaagtggcaccgagtcggtgctttt

SEQ ID NO: 250 - RNA, 100 nt, synthetic construct
  source 1..100: mol_type = other RNA
gcccgctgctgatgacacctgttttagagctagaaatagcaagttaaaataaggctagtccgttatcaacttgaaaaagtggcaccgagtcggtgctttt

SEQ ID NO: 251 - RNA, 100 nt, synthetic construct
  source 1..100: mol_type = other RNA
gtttgctgctgatgacacctgttttagagctagaaatagcaagttaaaataaggctagtccgttatcaacttgaaaaagtggcaccgagtcggtgctttt

SEQ ID NO: 252 - RNA, 100 nt, synthetic construct
  source 1..100: mol_type = other RNA
aaagttctagatgctgtccggttttagagctagaaatagcaagttaaaataaggctagtccgttatcaacttgaaaaagtggcaccgagtcggtgctttt

SEQ ID NO: 253 - RNA, 100 nt, synthetic construct
  source 1..100: mol_type = other RNA
gggcggcaatggtgtagcgggttttagagctagaaatagcaagttaaaataaggctagtccgttatcaacttgaaaaagtggcaccgagtcggtgctttt

SEQ ID NO: 254 - RNA, 100 nt, synthetic construct
  source 1..100: mol_type = other RNA
cattgatggcaggactgcctgttttagagctagaaatagcaagttaaaataaggctagtccgttatcaacttgaaaaagtggcaccgagtcggtgctttt

SEQ ID NO: 255 - RNA, 100 nt, synthetic construct
  source 1..100: mol_type = other RNA
aaaggctgctgatgacacctgttttagagctagaaatagcaagttaaaataaggctagtccgttatcaacttgaaaaagtggcaccgagtcggtgctttt

SEQ ID NO: 256 - RNA, 22 nt, synthetic construct
  source 1..22: mol_type = other RNA
gttttagagctatgctgttttg

SEQ ID NO: 257 - RNA, 74 nt, synthetic construct
  source 1..74: mol_type = other RNA
aacagcatagcaagttaaaataaggctagtccgttatcaacttgaaaaagtggcaccgagtcggtgcttttttt

SEQ ID NO: 258 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 259 - RNA, 12 nt, synthetic construct
  source 1..12: mol_type = other RNA
gttttagagcta

SEQ ID NO: 260 - RNA, 65 nt, synthetic construct
  source 1..65: mol_type = other RNA
atagcaagttaaaataaggctagtccgttatcaacttgaaaaagtggcaccgagtcggtgctttt

SEQ ID NO: 261 - RNA, 100 nt, synthetic construct
  source 1..100: mol_type = other RNA
  modified_base order(1..3,29..40,69..100): mod_base = OTHER; note = 2-prime-O-methyl nucleotides
  modified_base order(1..3,97..100): mod_base = OTHER; note = Phosphorothioate linkages
nnnnnnnnnnnnnnnnnnnngttttagagctagaaatagcaagttaaaataaggctagtccgttatcaacttgaaaaagtggcaccgagtcggtgctttt

SEQ ID NO: 262-271 - skipped (10 entries)

SEQ ID NO: 272 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 273 - RNA, 4140 nt, synthetic construct
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 274 - RNA, 4134 nt, synthetic construct
  source 1..4134: mol_type = other RNA
gacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtc

SEQ ID NO: 275 - RNA, 20 nt, synthetic construct
  source 1..20: mol_type = other RNA
acacaaataccagtccagca

SEQ ID NO: 276 - RNA, 20 nt, synthetic construct
  source 1..20: mol_type = other RNA
tctagaactttgaccatcag

SEQ ID NO: 277 - RNA, 20 nt, synthetic construct
  source 1..20: mol_type = other RNA
ttacagccacgtctacagca

SEQ ID NO: 278 - RNA, 20 nt, synthetic construct
  source 1..20: mol_type = other RNA
acacaaataccagtccagcg

SEQ ID NO: 279 - RNA, 20 nt, synthetic construct
  source 1..20: mol_type = other RNA
  modified_base 7: mod_base = OTHER; note = Thymine
aaaggctgctgatgacacct

SEQ ID NO: 280 - RNA, 20 nt, synthetic construct
  source 1..20: mol_type = other RNA
  modified_base 10: mod_base = OTHER; note = Thymine
aaaggctgctgatgacacct

SEQ ID NO: 281 - RNA, 20 nt, synthetic construct
  source 1..20: mol_type = other RNA
  modified_base 13: mod_base = OTHER; note = Thymine
aaaggctgctgatgacacct

SEQ ID NO: 282 - RNA, 20 nt, synthetic construct
  source 1..20: mol_type = other RNA
  modified_base 20: mod_base = OTHER; note = Thymine
aaaggctgctgatgacacct

SEQ ID NO: 283 - RNA, 20 nt, synthetic construct
  source 1..20: mol_type = other RNA
  modified_base order(13,20): mod_base = OTHER; note = Thymine
aaaggctgctgatgacacct

SEQ ID NO: 284 - RNA, 20 nt, synthetic construct
  source 1..20: mol_type = other RNA
  modified_base order(7,10,13,20): mod_base = OTHER; note = Thymine
aaaggctgctgatgacacct

SEQ ID NO: 285 - RNA, 19 nt, synthetic construct
  source 1..19: mol_type = other RNA
aaggctgctgatgacacct

SEQ ID NO: 286 - RNA, 18 nt, synthetic construct
  source 1..18: mol_type = other RNA
aggctgctgatgacacct

SEQ ID NO: 287 - RNA, 17 nt, synthetic construct
  source 1..17: mol_type = other RNA
ggctgctgatgacacct

SEQ ID NO: 288 - RNA, 16 nt, synthetic construct
  source 1..16: mol_type = other RNA
gctgctgatgacacct

SEQ ID NO: 289 - RNA, 19 nt, synthetic construct
  source 1..19: mol_type = other RNA
  modified_base order(6,9,12,19): mod_base = OTHER; note = Thymine
aaggctgctgatgacacct

SEQ ID NO: 290 - RNA, 18 nt, synthetic construct
  source 1..18: mol_type = other RNA
  modified_base order(5,8,11,18): mod_base = OTHER; note = Thymine
aggctgctgatgacacct

SEQ ID NO: 291 - RNA, 17 nt, synthetic construct
  source 1..17: mol_type = other RNA
  modified_base order(4,7,10,17): mod_base = OTHER; note = Thymine
ggctgctgatgacacct

SEQ ID NO: 292 - RNA, 16 nt, synthetic construct
  source 1..16: mol_type = other RNA
  modified_base order(3,6,9,16): mod_base = OTHER; note = Thymine
gctgctgatgacacct

SEQ ID NO: 293 - RNA, 20 nt, synthetic construct
  source 1..20: mol_type = other RNA
  modified_base 1: mod_base = OTHER; note = Thymine
tgtgtctgaggctggcccta

SEQ ID NO: 294 - RNA, 20 nt, synthetic construct
  source 1..20: mol_type = other RNA
  modified_base 3: mod_base = OTHER; note = Thymine
tgtgtctgaggctggcccta

SEQ ID NO: 295 - RNA, 20 nt, synthetic construct
  source 1..20: mol_type = other RNA
  modified_base 5: mod_base = OTHER; note = Thymine
tgtgtctgaggctggcccta

SEQ ID NO: 296 - RNA, 20 nt, synthetic construct
  source 1..20: mol_type = other RNA
  modified_base 7: mod_base = OTHER; note = Thymine
tgtgtctgaggctggcccta

SEQ ID NO: 297 - RNA, 20 nt, synthetic construct
  source 1..20: mol_type = other RNA
  modified_base 13: mod_base = OTHER; note = Thymine
tgtgtctgaggctggcccta

SEQ ID NO: 298 - RNA, 20 nt, synthetic construct
  source 1..20: mol_type = other RNA
  modified_base 19: mod_base = OTHER; note = Thymine
tgtgtctgaggctggcccta

SEQ ID NO: 299 - RNA, 20 nt, synthetic construct
  source 1..20: mol_type = other RNA
  modified_base order(1,3,5,7): mod_base = OTHER; note = Thymine
tgtgtctgaggctggcccta

SEQ ID NO: 300 - RNA, 20 nt, synthetic construct
  source 1..20: mol_type = other RNA
  modified_base order(1,3,5,7,13): mod_base = OTHER; note = Thymine
tgtgtctgaggctggcccta

SEQ ID NO: 301 - RNA, 20 nt, synthetic construct
  source 1..20: mol_type = other RNA
  modified_base order(1,3,5,7,13,19): mod_base = OTHER; note = Thymine
tgtgtctgaggctggcccta

SEQ ID NO: 302 - RNA, 20 nt, synthetic construct
  source 1..20: mol_type = other RNA
  modified_base 8: mod_base = OTHER; note = Thymine
acacaaataccagtccagca

SEQ ID NO: 303 - RNA, 20 nt, synthetic construct
  source 1..20: mol_type = other RNA
  modified_base order(8,14): mod_base = OTHER; note = Thymine
acacaaataccagtccagca

SEQ ID NO: 304 - RNA, 20 nt, synthetic construct
  source 1..20: mol_type = other RNA
  modified_base order(8..11,13..14): mod_base = OTHER; note = 2-prime-fluoro nucleotides
tgtgtctgaggctggcccta

SEQ ID NO: 305 - RNA, 20 nt, synthetic construct
  source 1..20: mol_type = other RNA
  modified_base order(8..11,14..15): mod_base = OTHER; note = 2-prime-fluoro nucleotides
cttggattcaccggtgccct

SEQ ID NO: 306 - RNA, 20 nt, synthetic construct
  source 1..20: mol_type = other RNA
  modified_base order(8..11,14..15): mod_base = OTHER; note = 2-prime-fluoro nucleotides
acttggattcaccggtgccc

SEQ ID NO: 307 - RNA, 20 nt, synthetic construct
  source 1..20: mol_type = other RNA
  modified_base order(8..11,13..14): mod_base = OTHER; note = 2-prime-fluoro nucleotides
aaaggctgctgatgacacct

SEQ ID NO: 308 - RNA, 42 nt, synthetic construct
  source 1..42: mol_type = other RNA
acacaaataccagtccagcagttttagagctatgctgttttg

SEQ ID NO: 309 - RNA, 42 nt, synthetic construct
  source 1..42: mol_type = other RNA
tctagaactttgaccatcaggttttagagctatgctgttttg

SEQ ID NO: 310 - RNA, 42 nt, synthetic construct
  source 1..42: mol_type = other RNA
ttacagccacgtctacagcagttttagagctatgctgttttg

SEQ ID NO: 311 - RNA, 42 nt, synthetic construct
  source 1..42: mol_type = other RNA
acacaaataccagtccagcggttttagagctatgctgttttg

SEQ ID NO: 312 - RNA, 42 nt, synthetic construct; identical to SEQ ID NO: 170
  source 1..42: mol_type = other RNA
  modified_base 7: mod_base = OTHER; note = Thymine

SEQ ID NO: 313 - RNA, 42 nt, synthetic construct; identical to SEQ ID NO: 170
  source 1..42: mol_type = other RNA
  modified_base 10: mod_base = OTHER; note = Thymine

SEQ ID NO: 314 - RNA, 42 nt, synthetic construct; identical to SEQ ID NO: 170
  source 1..42: mol_type = other RNA
  modified_base 13: mod_base = OTHER; note = Thymine

SEQ ID NO: 315 - RNA, 42 nt, synthetic construct; identical to SEQ ID NO: 170
  source 1..42: mol_type = other RNA
  modified_base 20: mod_base = OTHER; note = Thymine

SEQ ID NO: 316 - RNA, 42 nt, synthetic construct; identical to SEQ ID NO: 170
  source 1..42: mol_type = other RNA
  modified_base order(13,20): mod_base = OTHER; note = Thymine

SEQ ID NO: 317 - RNA, 42 nt, synthetic construct; identical to SEQ ID NO: 170
  source 1..42: mol_type = other RNA
  modified_base order(7,10,13,20): mod_base = OTHER; note = Thymine

SEQ ID NO: 318 - RNA, 41 nt, synthetic construct
  source 1..41: mol_type = other RNA
aaggctgctgatgacacctgttttagagctatgctgttttg

SEQ ID NO: 319 - RNA, 40 nt, synthetic construct
  source 1..40: mol_type = other RNA
aggctgctgatgacacctgttttagagctatgctgttttg

SEQ ID NO: 320 - RNA, 39 nt, synthetic construct
  source 1..39: mol_type = other RNA
ggctgctgatgacacctgttttagagctatgctgttttg

SEQ ID NO: 321 - RNA, 38 nt, synthetic construct
  source 1..38: mol_type = other RNA
gctgctgatgacacctgttttagagctatgctgttttg

SEQ ID NO: 322 - RNA, 41 nt, synthetic construct; identical to SEQ ID NO: 318
  source 1..41: mol_type = other RNA
  modified_base order(6,9,12,19): mod_base = OTHER; note = Thymine

SEQ ID NO: 323 - RNA, 40 nt, synthetic construct; identical to SEQ ID NO: 319
  source 1..40: mol_type = other RNA
  modified_base order(5,8,11,18): mod_base = OTHER; note = Thymine

SEQ ID NO: 324 - RNA, 39 nt, synthetic construct; identical to SEQ ID NO: 320
  source 1..39: mol_type = other RNA
  modified_base order(4,7,10,17): mod_base = OTHER; note = Thymine

SEQ ID NO: 325 - RNA, 38 nt, synthetic construct; identical to SEQ ID NO: 321
  source 1..38: mol_type = other RNA
  modified_base order(3,6,9,16): mod_base = OTHER; note = Thymine

SEQ ID NO: 326 - RNA, 42 nt, synthetic construct; identical to SEQ ID NO: 133
  source 1..42: mol_type = other RNA
  modified_base 1: mod_base = OTHER; note = Thymine

SEQ ID NO: 327 - RNA, 42 nt, synthetic construct; identical to SEQ ID NO: 133
  source 1..42: mol_type = other RNA
  modified_base 3: mod_base = OTHER; note = Thymine

SEQ ID NO: 328 - RNA, 42 nt, synthetic construct; identical to SEQ ID NO: 133
  source 1..42: mol_type = other RNA
  modified_base 5: mod_base = OTHER; note = Thymine

SEQ ID NO: 329 - RNA, 42 nt, synthetic construct; identical to SEQ ID NO: 133
  source 1..42: mol_type = other RNA
  modified_base 7: mod_base = OTHER; note = Thymine

SEQ ID NO: 330 - RNA, 42 nt, synthetic construct; identical to SEQ ID NO: 133
  source 1..42: mol_type = other RNA
  modified_base 13: mod_base = OTHER; note = Thymine

SEQ ID NO: 331 - RNA, 42 nt, synthetic construct; identical to SEQ ID NO: 133
  source 1..42: mol_type = other RNA
  modified_base 19: mod_base = OTHER; note = Thymine

SEQ ID NO: 332 - RNA, 42 nt, synthetic construct; identical to SEQ ID NO: 133
  source 1..42: mol_type = other RNA
  modified_base order(1,3,5,7): mod_base = OTHER; note = Thymine

SEQ ID NO: 333 - RNA, 42 nt, synthetic construct; identical to SEQ ID NO: 133
  source 1..42: mol_type = other RNA
  modified_base order(1,3,5,7,13): mod_base = OTHER; note = Thymine

SEQ ID NO: 334 - RNA, 42 nt, synthetic construct; identical to SEQ ID NO: 133
  source 1..42: mol_type = other RNA
  modified_base order(1,3,5,7,13,19): mod_base = OTHER; note = Thymine

SEQ ID NO: 335 - RNA, 42 nt, synthetic construct; identical to SEQ ID NO: 308
  source 1..42: mol_type = other RNA
  modified_base 8: mod_base = OTHER; note = Thymine

SEQ ID NO: 336 - RNA, 42 nt, synthetic construct; identical to SEQ ID NO: 308
  source 1..42: mol_type = other RNA
  modified_base order(8,14): mod_base = OTHER; note = Thymine

SEQ ID NO: 337 - RNA, 42 nt, synthetic construct; identical to SEQ ID NO: 133
  source 1..42: mol_type = other RNA
  modified_base order(8..11,13..14): mod_base = OTHER; note = 2-prime-fluoro nucleotides

SEQ ID NO: 338 - RNA, 42 nt, synthetic construct; identical to SEQ ID NO: 134
  source 1..42: mol_type = other RNA
  modified_base order(8..11,14..15): mod_base = OTHER; note = 2-prime-fluoro nucleotides

SEQ ID NO: 339 - RNA, 42 nt, synthetic construct; identical to SEQ ID NO: 135
  source 1..42: mol_type = other RNA
  modified_base order(8..11,14..15): mod_base = OTHER; note = 2-prime-fluoro nucleotides

SEQ ID NO: 340 - RNA, 42 nt, synthetic construct; identical to SEQ ID NO: 170
  source 1..42: mol_type = other RNA
  modified_base order(8..11,13..14): mod_base = OTHER; note = 2-prime-fluoro nucleotides

SEQ ID NO: 341 - RNA, 100 nt, synthetic construct
  source 1..100: mol_type = other RNA
acacaaataccagtccagcagttttagagctagaaatagcaagttaaaataaggctagtccgttatcaacttgaaaaagtggcaccgagtcggtgctttt

SEQ ID NO: 342 - RNA, 100 nt, synthetic construct
  source 1..100: mol_type = other RNA
tctagaactttgaccatcaggttttagagctagaaatagcaagttaaaataaggctagtccgttatcaacttgaaaaagtggcaccgagtcggtgctttt

SEQ ID NO: 343 - RNA, 100 nt, synthetic construct
  source 1..100: mol_type = other RNA
ttacagccacgtctacagcagttttagagctagaaatagcaagttaaaataaggctagtccgttatcaacttgaaaaagtggcaccgagtcggtgctttt

SEQ ID NO: 344 - RNA, 100 nt, synthetic construct
  source 1..100: mol_type = other RNA
acacaaataccagtccagcggttttagagctagaaatagcaagttaaaataaggctagtccgttatcaacttgaaaaagtggcaccgagtcggtgctttt

SEQ ID NO: 345 - RNA, 100 nt, synthetic construct; identical to SEQ ID NO: 255
  source 1..100: mol_type = other RNA
  modified_base 7: mod_base = OTHER; note = Thymine

SEQ ID NO: 346 - RNA, 100 nt, synthetic construct; identical to SEQ ID NO: 255
  source 1..100: mol_type = other RNA
  modified_base 10: mod_base = OTHER; note = Thymine

SEQ ID NO: 347 - RNA, 100 nt, synthetic construct; identical to SEQ ID NO: 255
  source 1..100: mol_type = other RNA
  modified_base 13: mod_base = OTHER; note = Thymine

SEQ ID NO: 348 - RNA, 100 nt, synthetic construct; identical to SEQ ID NO: 255
  source 1..100: mol_type = other RNA
  modified_base 20: mod_base = OTHER; note = Thymine

SEQ ID NO: 349 - RNA, 100 nt, synthetic construct; identical to SEQ ID NO: 255
  source 1..100: mol_type = other RNA
  modified_base order(13,20): mod_base = OTHER; note = Thymine

SEQ ID NO: 350 - RNA, 100 nt, synthetic construct; identical to SEQ ID NO: 255
  source 1..100: mol_type = other RNA
  modified_base order(7,10,13,20): mod_base = OTHER; note = Thymine

SEQ ID NO: 351 - RNA, 99 nt, synthetic construct
  source 1..99: mol_type = other RNA
aaggctgctgatgacacctgttttagagctagaaatagcaagttaaaataaggctagtccgttatcaacttgaaaaagtggcaccgagtcggtgctttt

SEQ ID NO: 352 - RNA, 98 nt, synthetic construct
  source 1..98: mol_type = other RNA
aggctgctgatgacacctgttttagagctagaaatagcaagttaaaataaggctagtccgttatcaacttgaaaaagtggcaccgagtcggtgctttt

SEQ ID NO: 353 - RNA, 97 nt, synthetic construct
  source 1..97: mol_type = other RNA
ggctgctgatgacacctgttttagagctagaaatagcaagttaaaataaggctagtccgttatcaacttgaaaaagtggcaccgagtcggtgctttt

SEQ ID NO: 354 - RNA, 96 nt, synthetic construct
  source 1..96: mol_type = other RNA
gctgctgatgacacctgttttagagctagaaatagcaagttaaaataaggctagtccgttatcaacttgaaaaagtggcaccgagtcggtgctttt

SEQ ID NO: 355 - RNA, 99 nt, synthetic construct; identical to SEQ ID NO: 351
  source 1..99: mol_type = other RNA
  modified_base order(6,9,12,19): mod_base = OTHER; note = Thymine

SEQ ID NO: 356 - RNA, 98 nt, synthetic construct; identical to SEQ ID NO: 352
  source 1..98: mol_type = other RNA
  modified_base order(5,8,11,18): mod_base = OTHER; note = Thymine

SEQ ID NO: 357 - RNA, 97 nt, synthetic construct; identical to SEQ ID NO: 353
  source 1..97: mol_type = other RNA
  modified_base order(4,7,10,17): mod_base = OTHER; note = Thymine

SEQ ID NO: 358 - RNA, 96 nt, synthetic construct; identical to SEQ ID NO: 354
  source 1..96: mol_type = other RNA
  modified_base order(3,6,9,16): mod_base = OTHER; note = Thymine

SEQ ID NO: 359 - RNA, 100 nt, synthetic construct; identical to SEQ ID NO: 218
  source 1..100: mol_type = other RNA
  modified_base 1: mod_base = OTHER; note = Thymine

SEQ ID NO: 360 - RNA, 100 nt, synthetic construct; identical to SEQ ID NO: 218
  source 1..100: mol_type = other RNA
  modified_base 3: mod_base = OTHER; note = Thymine

SEQ ID NO: 361 - RNA, 100 nt, synthetic construct; identical to SEQ ID NO: 218
  source 1..100: mol_type = other RNA
  modified_base 5: mod_base = OTHER; note = Thymine

SEQ ID NO: 362 - RNA, 100 nt, synthetic construct; identical to SEQ ID NO: 218
  source 1..100: mol_type = other RNA
  modified_base 7: mod_base = OTHER; note = Thymine

SEQ ID NO: 363 - RNA, 100 nt, synthetic construct; identical to SEQ ID NO: 218
  source 1..100: mol_type = other RNA
  modified_base 13: mod_base = OTHER; note = Thymine

SEQ ID NO: 364 - RNA, 100 nt, synthetic construct; identical to SEQ ID NO: 218
  source 1..100: mol_type = other RNA
  modified_base 19: mod_base = OTHER; note = Thymine

SEQ ID NO: 365 - RNA, 100 nt, synthetic construct; identical to SEQ ID NO: 218
  source 1..100: mol_type = other RNA
  modified_base order(1,3,5,7): mod_base = OTHER; note = Thymine

SEQ ID NO: 366 - RNA, 100 nt, synthetic construct; identical to SEQ ID NO: 218
  source 1..100: mol_type = other RNA
  modified_base order(1,3,5,7,13): mod_base = OTHER; note = Thymine

SEQ ID NO: 367 - RNA, 100 nt, synthetic construct; identical to SEQ ID NO: 218
  source 1..100: mol_type = other RNA
  modified_base order(1,3,5,7,13,19): mod_base = OTHER; note = Thymine

SEQ ID NO: 368 - RNA, 100 nt, synthetic construct; identical to SEQ ID NO: 341
  source 1..100: mol_type = other RNA
  modified_base 8: mod_base = OTHER; note = Thymine

SEQ ID NO: 369 - RNA, 100 nt, synthetic construct; identical to SEQ ID NO: 341
  source 1..100: mol_type = other RNA
  modified_base order(8,14): mod_base = OTHER; note = Thymine

SEQ ID NO: 370 - RNA, 100 nt, synthetic construct; identical to SEQ ID NO: 218
  source 1..100: mol_type = other RNA
  modified_base order(8..11,13..14): mod_base = OTHER; note = 2-prime-fluoro nucleotides

SEQ ID NO: 371 - RNA, 100 nt, synthetic construct; identical to SEQ ID NO: 219
  source 1..100: mol_type = other RNA
  modified_base order(8..11,14..15): mod_base = OTHER; note = 2-prime-fluoro nucleotides

SEQ ID NO: 372 - RNA, 100 nt, synthetic construct; identical to SEQ ID NO: 220
  source 1..100: mol_type = other RNA
  modified_base order(8..11,14..15): mod_base = OTHER; note = 2-prime-fluoro nucleotides

SEQ ID NO: 373 - RNA, 100 nt, synthetic construct; identical to SEQ ID NO: 255
  source 1..100: mol_type = other RNA
  modified_base order(8..11,13..14): mod_base = OTHER; note = 2-prime-fluoro nucleotides

SEQ ID NO: 374 - protein, 5 aa, synthetic construct
GGGGS